SEQUENCE LISTING (60 sequences)

SEQ ID NO: 1 - protein, 1807 aa, Homo sapiens
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCDYMEPERGSEKKTSGPLSPPTGPPGPAPAGPAVRLGSVPYSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSFDDTPEKDSFRARSTSLNERPKSLRIARPPKQGLNNSPPVKEFKESSAAEAFRCRSISVSEHVVRSRIQTSLTSASLGSADENSVAQADDSLKNLHLELTETCLDMMARYVFSNFTAVPKRSPVGEFLLAGGRTKTWLVGNKLVTVTTSVGTGTRSLLGLDSGELQSGPESSSSPGVHVRQTKEAPAKLESQAGQQVSRGARDRVRSMSGGHGLRVGALDVPASQFLGSATSPGPRTAPAAKPEKASAGTRVPVQEKTNLAAYVPLLTQGWAEILVRRPTGNTSWLMSLENPLSPFSSDINNMPLQELSNALMAAERFKEHRDTALYKSLSVPAASTAKPPPLPRSNTVASFSSLYQSSCQGQLHRSVSWADSAVVMEEGSPGEVPVLVEPPGLEDVEAALGMDRRTDAYSRSSSVSSQEEKSLHAEELVGRGIPIERVVSSEGGRPSVDLSFQPSQPLSKSSSSPELQTLQDILGDPGDKADVGRLSPEVKARSQSGTLDGESAAWSASGEDSRGQPEGPLPSSSPRSPSGLRPRGYTISDSAPSRRGKRVERDALKSRATASNAEKVPGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPSLPLVHPPSHSKAPAQTPAEPTPGYEVGQRKRLISSVEDFTEFV

SEQ ID NO: 2 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 850 bp, synthetic construct
  source 1..850: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgct

SEQ ID NO: 4 - DNA, 225 bp, synthetic construct
  source 1..225: mol_type = other DNA
ggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggc

SEQ ID NO: 5 - DNA, 269 bp, synthetic construct
  source 1..269: mol_type = other DNA
acccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgc

SEQ ID NO: 6 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 2955 bp, synthetic construct
  source 1..2955: mol_type = other DNA
atgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgggcagttcgggcagctccgggtcctccggctcctctatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggatccggaaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcggcagcggagggctggcggcctatgtgcccctgctgacccagggctgggcggagatcctggtccggaggcccacagggaacaccagctggctgatgagcctggagaacccgggatctggtatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 8 - DNA, 2874 bp, synthetic construct
  source 1..2874: mol_type = other DNA
agagccctgttcttcaaggtgatcaaggactacccaagcaatgaagacctgcatgagcgcctggaggtgttcaaagccctgaccgataatggccggcacatcacctacctggaggaggaactggcagactttgtcctgcaatggatggatgtggggctgagctctgagtttctgctggtgctggtgaatctggtgaagtttaacagctgctacctggatgagtatatcgccaggatggtgcagatgatttgcctgctgtgtgtgcgcaccgccagctcagtggacattgaagtgtctctgcaggtgctggatgccgtggtgtgctacaactgcctgcccgctgagtccctgcccctgttcatcgtgactctgtgccggaccatcaatgtcaaggagctgtgcgagccttgctggaaactgatgagaaatctgctgggcacacatctggggcacagcgctatctacaacatgtgccacctgatggaagatcgcgcttatatggaagacgcccctctgctgaggggagccgtgttcttcgtgggaatggccctgtggggagcccacaggctctactccctgcggaacagccccacctctgtgctgccgtccttctatcaggccatggcctgtcccaacgaggtggtgtcttacgagatagtgctgtccatcaccagactgattaagaaatacaggaaggagctgcaggtggtggcctgggatattctcctgaatatcatcgagcggctgctgcaacagctgcagaccctcgattctcctgagctgcggaccatcgtgcacgacctgctgaccaccgtggaggaactgtgcgaccagaacgaattccacgggtctcaggagaggtacttcgagctcgttgagagatgtgctgatcagagacccgagtcctccctcctgaacctgattagctacagagcccagtccattcatccagctaaagatgggtggattcagaacctgcaggcccttatggagcggttcttccggtcagagtcaagaggcgccgttcggattaaagtcctggacgtgctgagctttgtgctgctgatcaatagacagttctatgaggaagagctgatcaactccgtggtgattagccagctgagccacatccccgaggacaaagaccaccaggtgaggaagctggcgacacagctgctggtggacctggccgagggctgtcacacacatcacttcaacagcctcctggatatcatcgagaaagtgatggcaaggtccctgagccccccccccgaactggaggaaagggacgtggccgcttactcagccagcctggaggacgtgaagactgccgtgctgggcctgctggtgattctgcagaccaagctcggcagttcgggcagctccgggtcctccggctcctctatgcccgacattatcatcaaagccctgcctgtgctggtggtgaagctgacacacatcagcgccaccgcttcaatggccgtgcctctgctggaatttctgagcacactggcccggctgccccacctgtatcggaacttcgccgccgagcagtacgccagcgtgtttgccatcagcctgccttacacaaacccctccaaatttaaccagtacatcgtctgcctggctcaccacgtgatcgccatgtggttcatccgatgcagactgccctttagaaaggacttcgtgcctttcatcaccaaggggctgaggagcaacgtgctgctgagcggatccggaaagaacctgcatctggagctgaccgagacttgcctggacatgatggccagatacgtgttctccaatttcacagctgtgccaaagaggagccccgtgggcgagttcttgctggccggaggcagaacaaagacctggctggtcggcaacaagcttgtgaccgtgaccactagcgtcggcaccggaaccagaagcctgctgggcggcagcggagggctcgccgcctatgtgccactgctgacgcaaggatgggccgagatcctggtccggaggccaaccggcaacacatcctggctgatgtcactggagaatcccggatctggtatcaatccaagtttcgtgttcctgcagctgtaccacagccccttcttcggcgacgagtccaataagcctatcctgctgcctaacgagtcccagagctttgagcgcagcgtgcaactgttggaccagatccccagctatgacactcacaagatcgccgtgctgtatgtgggcgagggacagagcaactcagagctggccatcctgtccaacgagcacggcagctacagatacaccgagttcctgaccggcctgggccgcctgatcgagctgaaagactgccagccagataaggtgtatctgggcggcctggacgtgtgcggcgaggacggacagtttacatactgctggcatgacgatattatgcaggccgtgttccacattgccacactgatgccaaccaaagacgtggacaagcacaggtgcgataagaagagacacctgggtaatgacttcgtttcaatcgtgtacaatgatagcggcgaggacttcaaactgggcaccatcaagggccaatttaattttgtgcacgtcatcgtgacacccctggactacgagtgcaacctcgtgtcactgcagtgtcggaaggacatggaaggcctggtggatacctcagtggctaagattgtgtccgaccgcaacctgccattcgtggcccgacagatggccctgcatgccaatatggccagccaggtgcaccattcacgctccaatcctactgatatctacccctctaagtggatcgccaggctgaggcacatcaagagactgcggcagcggatctgtgaggaggccgcctatagcaaccctagctga

SEQ ID NO: 9 - DNA, 4616 bp, synthetic construct
  source 1..4616: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgggcagttcgggcagctccgggtcctccggctcctctatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggatccggaaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcggcagcggagggctggcggcctatgtgcccctgctgacccagggctgggcggagatcctggtccggaggcccacagggaacaccagctggctgatgagcctggagaacccgggatctggtatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 10 - DNA, 4616 bp, synthetic construct
  source 1..4616: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgcctctggaggctaggcacgccgtgctggctctgctcaaggccatcgtgcagggccagggtgagcggctgggcgtgctgagagccctgttcttcaaggtgatcaaggactacccaagcaatgaagacctgcatgagcgcctggaggtgttcaaagccctgaccgataatggccggcacatcacctacctggaggaggaactggcagactttgtcctgcaatggatggatgtggggctgagctctgagtttctgctggtgctggtgaatctggtgaagtttaacagctgctacctggatgagtatatcgccaggatggtgcagatgatttgcctgctgtgtgtgcgcaccgccagctcagtggacattgaagtgtctctgcaggtgctggatgccgtggtgtgctacaactgcctgcccgctgagtccctgcccctgttcatcgtgactctgtgccggaccatcaatgtcaaggagctgtgcgagccttgctggaaactgatgagaaatctgctgggcacacatctggggcacagcgctatctacaacatgtgccacctgatggaagatcgcgcttatatggaagacgcccctctgctgaggggagccgtgttcttcgtgggaatggccctgtggggagcccacaggctctactccctgcggaacagccccacctctgtgctgccgtccttctatcaggccatggcctgtcccaacgaggtggtgtcttacgagatagtgctgtccatcaccagactgattaagaaatacaggaaggagctgcaggtggtggcctgggatattctcctgaatatcatcgagcggctgctgcaacagctgcagaccctcgattctcctgagctgcggaccatcgtgcacgacctgctgaccaccgtggaggaactgtgcgaccagaacgaattccacgggtctcaggagaggtacttcgagctcgttgagagatgtgctgatcagagacccgagtcctccctcctgaacctgattagctacagagcccagtccattcatccagctaaagatgggtggattcagaacctgcaggcccttatggagcggttcttccggtcagagtcaagaggcgccgttcggattaaagtcctggacgtgctgagctttgtgctgctgatcaatagacagttctatgaggaagagctgatcaactccgtggtgattagccagctgagccacatccccgaggacaaagaccaccaggtgaggaagctggcgacacagctgctggtggacctggccgagggctgtcacacacatcacttcaacagcctcctggatatcatcgagaaagtgatggcaaggtccctgagccccccccccgaactggaggaaagggacgtggccgcttactcagccagcctggaggacgtgaagactgccgtgctgggcctgctggtgattctgcagaccaagctcggcagttcgggcagctccgggtcctccggctcctctatgcccgacattatcatcaaagccctgcctgtgctggtggtgaagctgacacacatcagcgccaccgcttcaatggccgtgcctctgctggaatttctgagcacactggcccggctgccccacctgtatcggaacttcgccgccgagcagtacgccagcgtgtttgccatcagcctgccttacacaaacccctccaaatttaaccagtacatcgtctgcctggctcaccacgtgatcgccatgtggttcatccgatgcagactgccctttagaaaggacttcgtgcctttcatcaccaaggggctgaggagcaacgtgctgctgagcggatccggaaagaacctgcatctggagctgaccgagacttgcctggacatgatggccagatacgtgttctccaatttcacagctgtgccaaagaggagccccgtgggcgagttcttgctggccggaggcagaacaaagacctggctggtcggcaacaagcttgtgaccgtgaccactagcgtcggcaccggaaccagaagcctgctgggcggcagcggagggctcgccgcctatgtgccactgctgacgcaaggatgggccgagatcctggtccggaggccaaccggcaacacatcctggctgatgtcactggagaatcccggatctggtatcaatccaagtttcgtgttcctgcagctgtaccacagccccttcttcggcgacgagtccaataagcctatcctgctgcctaacgagtcccagagctttgagcgcagcgtgcaactgttggaccagatccccagctatgacactcacaagatcgccgtgctgtatgtgggcgagggacagagcaactcagagctggccatcctgtccaacgagcacggcagctacagatacaccgagttcctgaccggcctgggccgcctgatcgagctgaaagactgccagccagataaggtgtatctgggcggcctggacgtgtgcggcgaggacggacagtttacatactgctggcatgacgatattatgcaggccgtgttccacattgccacactgatgccaaccaaagacgtggacaagcacaggtgcgataagaagagacacctgggtaatgacttcgtttcaatcgtgtacaatgatagcggcgaggacttcaaactgggcaccatcaagggccaatttaattttgtgcacgtcatcgtgacacccctggactacgagtgcaacctcgtgtcactgcagtgtcggaaggacatggaaggcctggtggatacctcagtggctaagattgtgtccgaccgcaacctgccattcgtggcccgacagatggccctgcatgccaatatggccagccaggtgcaccattcacgctccaatcctactgatatctacccctctaagtggatcgccaggctgaggcacatcaagagactgcggcagcggatctgtgaggaggccgcctatagcaaccctagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - protein, 984 aa, synthetic construct
MPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLGSSGSSGSSGSSMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGGSGGLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 12 - DNA, 2961 bp, synthetic construct
  source 1..2961: mol_type = other DNA
atgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccggctcctctggtagctctggaagttccggctccagcggctcctccggatctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagcgggtccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctcaggaaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggctccagtggcagcctggcggcctatgtgcccctgctgacccagggctgggcggagatcctggtccggaggcccacagggaacaccagctggctgatgagcctggagaacccgggatccggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 13 - DNA, 2961 bp, synthetic construct
  source 1..2961: mol_type = other DNA
atgcctctggaggctcggcatgccgtgctggccctgctgaaggccattgtgcaaggccagggcgaaaggttgggggtgctgagggccctgttctttaaggtgatcaaggactatccctccaacgaggacttgcacgagaggctggaagtgtttaaggccctgactgataacggacgccacatcacctatctcgaggaagaactggctgactttgtgttgcagtggatggatgtcggcctgtcaagcgagttcctcctggtgctggtgaacctggtgaagttcaattcctgctacctggatgaatacatcgcccggatggtgcagatgatctgcctgctgtgcgtgagaaccgcctcatctgtggatatagaggtgagcctccaggtgctggacgccgtggtgtgctataattgcctgccagccgaaagcctgccactgttcatcgtgacactgtgtaggaccattaatgtgaaggagctgtgcgagccatgctggaagctcatgaggaatctgctgggaacacacctgggccacagcgctatctacaacatgtgccacctgatggaggaccgggcgtacatggaggacgccccactgctgcggggcgccgtgttcttcgtgggaatggccctgtggggggctcacagactgtactcactgcggaatagccctacctctgtgctgccttctttttatcaggctatggcctgtcccaatgaggtggtgtcttacgagattgtgctgtctattaccagactgattaagaagtataggaaagagctgcaggtcgtggcctgggacattctcctgaacatcatcgagcggctgctgcagcagctgcagacaggctcctctggtagctctggaagttccggctccagcggctcctccggatctgcctctcacgccacccgggtgtacgaaatgctggtgtcccacatccagctgcattataagcacagttataccctgcctattgccagcagcatccggctgcaggccttcgatttcctcctgctgctgcgcgccgatagcctgcaccgccttggactgccaaacaaggatggcgtggtgagattctccccttactgcgtgtgcggcagcgggtccctgctgtttagggttctgctgcagtgtctgaagcaggagtcagactggaaagtgctgaagctggtgctgggaagactgccagagagcctgcgatacaaggtgctgatcttcaccagcccatgctccgtggatcagctgtgttccgccctctgcagcatgctgagcggtcctaagaccctggaacggctgaggggcgccccagagggcttcagccgcactgatctgcacctggctgtggtgcccgtgctgaccgccctgattagctaccataactatctggacaagaccaagcagagggagatggtgtactgcctggaacagggactgatccacagatgtgcttcccagtgtgtggtggccctgtccatttgctctgtggagatgcctgacatcattatcaaagccctgccagtgctggtggtgaaactgactcacatctccgccacggcctctatggccgtccctctcctggagttcctgtccaccctcgcccggctgcctcacctgtacaggaactttgctgccgagcagtacgcaagcgtttttgccatcagcctgccttacaccaatccttctaagttcaaccagtacatcgtgtgcctggcccaccatgtgattgccatgtggtttatccgctgccgcctgccctttcggaaggacttcgtgcccttcatcaccaaggggctgaggtctaatgttctgctgtccggctcaggaaagaacctgcaccttgaactgacagaaacctgcctggatatgatggcccggtacgtgttttccaatttcacggctgtgcccaagcggagccctgtcggcgagttcctgctggccggaggcaggaccaagacatggctggtgggcaacaaactggtgaccgtgaccaccagcgtgggcacaggcaccaggtcactgctgggctccagtggcagcctggctgcctacgtgcccctgctgacacagggctgggctgaaatcctggtgaggagaccaaccggcaacacctcttggctgatgagcctggagaatcccggatccggcatcaatccaagcttcgtgtttctgcagctgtaccactcacccttctttggggacgagtccaacaaacccatcctgctgcccaacgagtcccagtcttttgagagaagtgtgcagctgctggaccagattccaagttacgacactcacaagatcgcagtgctgtatgtgggtgagggccagagcaacagcgaactggccatcctgtctaatgagcatggcagctacagatacaccgaatttctgaccggactgggcaggctgatcgagcttaaagactgtcagcccgacaaagtgtacctgggcggcctggacgtgtgcggggaagacggacagttcacctactgctggcacgacgacattatgcaggccgtgtttcacatcgccaccctgatgcctactaaagacgtggacaaacataggtgcgacaagaagcgccacctggggaacgactttgtgagcattgtgtacaacgactctggcgaggatttcaagctggggacaatcaaagggcagtttaacttcgtgcacgtgatcgtgactccactggactacgagtgtaatctggtgagcctgcagtgccggaaggacatggagggcctggtggacacctcagtggccaagatcgtgagcgatcgtaatctgccttttgtggccagacagatggccctgcacgccaacatggcctcccaggtgcaccactctcgctccaacccaaccgacatctacccatccaagtggatcgccagactgagacacattaagaggctcaggcaaagaatctgtgaggaagccgcctactccaatccctcctga

SEQ ID NO: 14 - DNA, 4622 bp, synthetic construct
  source 1..4622: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccggctcctctggtagctctggaagttccggctccagcggctcctccggatctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagcgggtccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctcaggaaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggctccagtggcagcctggcggcctatgtgcccctgctgacccagggctgggcggagatcctggtccggaggcccacagggaacaccagctggctgatgagcctggagaacccgggatccggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 4622 bp, synthetic construct
  source 1..4622: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgcctctggaggctcggcatgccgtgctggccctgctgaaggccattgtgcaaggccagggcgaaaggttgggggtgctgagggccctgttctttaaggtgatcaaggactatccctccaacgaggacttgcacgagaggctggaagtgtttaaggccctgactgataacggacgccacatcacctatctcgaggaagaactggctgactttgtgttgcagtggatggatgtcggcctgtcaagcgagttcctcctggtgctggtgaacctggtgaagttcaattcctgctacctggatgaatacatcgcccggatggtgcagatgatctgcctgctgtgcgtgagaaccgcctcatctgtggatatagaggtgagcctccaggtgctggacgccgtggtgtgctataattgcctgccagccgaaagcctgccactgttcatcgtgacactgtgtaggaccattaatgtgaaggagctgtgcgagccatgctggaagctcatgaggaatctgctgggaacacacctgggccacagcgctatctacaacatgtgccacctgatggaggaccgggcgtacatggaggacgccccactgctgcggggcgccgtgttcttcgtgggaatggccctgtggggggctcacagactgtactcactgcggaatagccctacctctgtgctgccttctttttatcaggctatggcctgtcccaatgaggtggtgtcttacgagattgtgctgtctattaccagactgattaagaagtataggaaagagctgcaggtcgtggcctgggacattctcctgaacatcatcgagcggctgctgcagcagctgcagacaggctcctctggtagctctggaagttccggctccagcggctcctccggatctgcctctcacgccacccgggtgtacgaaatgctggtgtcccacatccagctgcattataagcacagttataccctgcctattgccagcagcatccggctgcaggccttcgatttcctcctgctgctgcgcgccgatagcctgcaccgccttggactgccaaacaaggatggcgtggtgagattctccccttactgcgtgtgcggcagcgggtccctgctgtttagggttctgctgcagtgtctgaagcaggagtcagactggaaagtgctgaagctggtgctgggaagactgccagagagcctgcgatacaaggtgctgatcttcaccagcccatgctccgtggatcagctgtgttccgccctctgcagcatgctgagcggtcctaagaccctggaacggctgaggggcgccccagagggcttcagccgcactgatctgcacctggctgtggtgcccgtgctgaccgccctgattagctaccataactatctggacaagaccaagcagagggagatggtgtactgcctggaacagggactgatccacagatgtgcttcccagtgtgtggtggccctgtccatttgctctgtggagatgcctgacatcattatcaaagccctgccagtgctggtggtgaaactgactcacatctccgccacggcctctatggccgtccctctcctggagttcctgtccaccctcgcccggctgcctcacctgtacaggaactttgctgccgagcagtacgcaagcgtttttgccatcagcctgccttacaccaatccttctaagttcaaccagtacatcgtgtgcctggcccaccatgtgattgccatgtggtttatccgctgccgcctgccctttcggaaggacttcgtgcccttcatcaccaaggggctgaggtctaatgttctgctgtccggctcaggaaagaacctgcaccttgaactgacagaaacctgcctggatatgatggcccggtacgtgttttccaatttcacggctgtgcccaagcggagccctgtcggcgagttcctgctggccggaggcaggaccaagacatggctggtgggcaacaaactggtgaccgtgaccaccagcgtgggcacaggcaccaggtcactgctgggctccagtggcagcctggctgcctacgtgcccctgctgacacagggctgggctgaaatcctggtgaggagaccaaccggcaacacctcttggctgatgagcctggagaatcccggatccggcatcaatccaagcttcgtgtttctgcagctgtaccactcacccttctttggggacgagtccaacaaacccatcctgctgcccaacgagtcccagtcttttgagagaagtgtgcagctgctggaccagattccaagttacgacactcacaagatcgcagtgctgtatgtgggtgagggccagagcaacagcgaactggccatcctgtctaatgagcatggcagctacagatacaccgaatttctgaccggactgggcaggctgatcgagcttaaagactgtcagcccgacaaagtgtacctgggcggcctggacgtgtgcggggaagacggacagttcacctactgctggcacgacgacattatgcaggccgtgtttcacatcgccaccctgatgcctactaaagacgtggacaaacataggtgcgacaagaagcgccacctggggaacgactttgtgagcattgtgtacaacgactctggcgaggatttcaagctggggacaatcaaagggcagtttaacttcgtgcacgtgatcgtgactccactggactacgagtgtaatctggtgagcctgcagtgccggaaggacatggagggcctggtggacacctcagtggccaagatcgtgagcgatcgtaatctgccttttgtggccagacagatggccctgcacgccaacatggcctcccaggtgcaccactctcgctccaacccaaccgacatctacccatccaagtggatcgccagactgagacacattaagaggctcaggcaaagaatctgtgaggaagccgcctactccaatccctcctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 986 aa, synthetic construct
MPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTGSSGSSGSSGSSGSSGSASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGSSGSLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 17 - DNA, 2901 bp, synthetic construct
  source 1..2901: mol_type = other DNA
atgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagcggatctggatgcctgccggctgagagcctcccgctgttcatcgttaccctctgtcgcaccatcaacgtcaaggagctctgcgagccttgctggaagctgatgcggaacctccttggcacccacggctcctccggctctagcggcagcgtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctggcagctccggaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacgggagtggcgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagtgggtccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctccggtaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggaagctctggctctctggcggcctatgtgcccctgctgacccagggctgggcggagatcctggtccggaggcccacagggaacaccagctggctgatgagcctggagaacccgggctccggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 18 - DNA, 2901 bp, synthetic construct
  source 1..2901: mol_type = other DNA
atgcctctggaggctaggcacgccgtgctggccctgctgaaggccatcgtgcagggccagggcgagcgcttgggcgtgctgagagccctgttttttaaggtgattaaggactatccctccaacgaggatctgcacgagagactggaggtgttcaaagctctgacagacaacggaagacacatcacctatctggaggaggagctggccgactttgtgctgcagtggatggatgtgggactgagctctgagtttctgctggtgctggtgaatctggtgaagttcaacagcggatctggatgtctccctgccgagagcctgccactgtttatcgtgaccctgtgcaggaccatcaacgtgaaggaactgtgtgagccctgttggaagctgatgagaaacctgctgggtacccacggctcctccggctctagcggcagcgtggtggcttgggacatcctgttgaacatcattgaaagactgctgcagcagctccagactctggacagtcccgaacttaggacaatcgtgcacgacctactgaccaccgtggaggagctgtgcgatcagaacgagttccacggaagccaggagaggtatttcgaactggtggaaagatgcgctgaccagaggcctggcagctccggaggcgccgtgagaatcaaggtgctggatgtgctgagctttgtgctgctgattaaccggcagttctacgaggaggaactgattaatagcgttgtgatttcacagctctcacacattcccgaggataaggatcaccaggtcaggaagctggccacccagctcctggtggacctggccgagggttgccacgggagtggcgcctcacacgccacccgggtgtacgaaatgctcgtgagtcacatccagctgcactacaagcacagctatacactgcccatcgcttccagcattcgcctgcaggcctttgactttctgctgctgctgagagccgatagtctgcaccggctgggcctgcccaacaaggacggggtggtgaggttcagtccatattgcgtgtgcggcagtgggtcactgctgttcagggtgctgctgcagtgcctgaaacaggagtctgattggaaagtgctgaagctggtgctgggcagacttcccgagagcctgagatacaaggtgctgatcttcacctctccatgcagtgtggaccaactgtgtagcgccctgtgctccatgctgagcggcccaaagaccctggaacggctgagaggcgcccccgaaggatttagcaggaccgacctgcacctggctgtggtgcccgtcctgacagccctgattagctaccacaactacctggacaagacaaagcagagggagatggtgtactgcctggagcagggactgatccaccgctgtgccagccagtgcgtggtggccctgagcatctgttccgtggagatgcctgatattatcatcaaggccctgcccgtgctggtggtgaagctgacacacattagcgcaactgcttccatggccgtgcctctgctggagttcctgagtacactggcccggctgccccacctctatagaaactttgccgccgagcagtacgcctccgtgttcgctatctctctgccatacacaaacccctctaagttcaaccagtacatcgtgtgcctggcacaccacgttatcgccatgtggttcatccgctgtaggctgcccttccgcaaggattttgtgccgtttatcaccaagggcctgcggtccaacgtgctgctgagtggctccggtaagaacctccatctggagctgaccgagacctgtctggacatgatggcaaggtacgtgttcagcaacttcacagccgtgccaaagcggtcccccgtgggcgagtttctgcttgctggcgggcggacaaagacctggctcgtgggaaataagctggtgaccgttaccacctccgtgggaactggcaccagatctctgctcggaagctctggctctctggccgcctatgtgcccctgctgacccagggatgggccgaaatcctggtgcgaaggccaacaggcaacacctcctggctgatgagcctggagaatcctggctccggcatcaacccttctttcgtgttcctgcagctgtaccattcccccttcttcggcgacgagagcaacaagccaatcctgctgcctaacgagtcccagagcttcgagcgatcggtgcagctgctggaccagatccccagttatgacacccataaaatcgccgtgctgtacgtgggagagggccagagcaacagcgagctggccatcctgtcaaatgaacacggcagctacaggtatactgagttcctcacaggcctgggaagactgatcgagctgaaggattgccagcctgataaggtgtacctgggaggcctggacgtgtgcggtgaagacgggcagtttacctactgctggcatgatgatatcatgcaggcagtgttccatatcgccacactgatgcccaccaaggatgtggacaaacaccgctgcgataagaaaaggcacctgggcaacgacttcgtgagtatcgtgtacaacgacagcggggaggacttcaaactgggcaccattaaaggacagttcaattttgtccacgtgatcgtgactcccctggactacgagtgcaacctggtgagtctgcagtgccgcaaagatatggagggcctggtggacacctccgtggctaagatcgtgagcgacagaaacctgcctttcgtggcacggcagatggccctgcacgccaacatggccagccaggtgcaccactcaaggtccaaccccaccgacatctatcccagcaaatggatcgctcgcctgcggcacatcaaacggctcaggcagcggatctgtgaggaggctgcctatagcaaccccagttga

SEQ ID NO: 19 - DNA, 4562 bp, synthetic construct
  source 1..4562: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagcggatctggatgcctgccggctgagagcctcccgctgttcatcgttaccctctgtcgcaccatcaacgtcaaggagctctgcgagccttgctggaagctgatgcggaacctccttggcacccacggctcctccggctctagcggcagcgtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctggcagctccggaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacgggagtggcgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagtgggtccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctccggtaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggaagctctggctctctggcggcctatgtgcccctgctgacccagggctgggcggagatcctggtccggaggcccacagggaacaccagctggctgatgagcctggagaacccgggctccggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 4562 bp, synthetic construct
  source 1..4562: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgcctctggaggctaggcacgccgtgctggccctgctgaaggccatcgtgcagggccagggcgagcgcttgggcgtgctgagagccctgttttttaaggtgattaaggactatccctccaacgaggatctgcacgagagactggaggtgttcaaagctctgacagacaacggaagacacatcacctatctggaggaggagctggccgactttgtgctgcagtggatggatgtgggactgagctctgagtttctgctggtgctggtgaatctggtgaagttcaacagcggatctggatgtctccctgccgagagcctgccactgtttatcgtgaccctgtgcaggaccatcaacgtgaaggaactgtgtgagccctgttggaagctgatgagaaacctgctgggtacccacggctcctccggctctagcggcagcgtggtggcttgggacatcctgttgaacatcattgaaagactgctgcagcagctccagactctggacagtcccgaacttaggacaatcgtgcacgacctactgaccaccgtggaggagctgtgcgatcagaacgagttccacggaagccaggagaggtatttcgaactggtggaaagatgcgctgaccagaggcctggcagctccggaggcgccgtgagaatcaaggtgctggatgtgctgagctttgtgctgctgattaaccggcagttctacgaggaggaactgattaatagcgttgtgatttcacagctctcacacattcccgaggataaggatcaccaggtcaggaagctggccacccagctcctggtggacctggccgagggttgccacgggagtggcgcctcacacgccacccgggtgtacgaaatgctcgtgagtcacatccagctgcactacaagcacagctatacactgcccatcgcttccagcattcgcctgcaggcctttgactttctgctgctgctgagagccgatagtctgcaccggctgggcctgcccaacaaggacggggtggtgaggttcagtccatattgcgtgtgcggcagtgggtcactgctgttcagggtgctgctgcagtgcctgaaacaggagtctgattggaaagtgctgaagctggtgctgggcagacttcccgagagcctgagatacaaggtgctgatcttcacctctccatgcagtgtggaccaactgtgtagcgccctgtgctccatgctgagcggcccaaagaccctggaacggctgagaggcgcccccgaaggatttagcaggaccgacctgcacctggctgtggtgcccgtcctgacagccctgattagctaccacaactacctggacaagacaaagcagagggagatggtgtactgcctggagcagggactgatccaccgctgtgccagccagtgcgtggtggccctgagcatctgttccgtggagatgcctgatattatcatcaaggccctgcccgtgctggtggtgaagctgacacacattagcgcaactgcttccatggccgtgcctctgctggagttcctgagtacactggcccggctgccccacctctatagaaactttgccgccgagcagtacgcctccgtgttcgctatctctctgccatacacaaacccctctaagttcaaccagtacatcgtgtgcctggcacaccacgttatcgccatgtggttcatccgctgtaggctgcccttccgcaaggattttgtgccgtttatcaccaagggcctgcggtccaacgtgctgctgagtggctccggtaagaacctccatctggagctgaccgagacctgtctggacatgatggcaaggtacgtgttcagcaacttcacagccgtgccaaagcggtcccccgtgggcgagtttctgcttgctggcgggcggacaaagacctggctcgtgggaaataagctggtgaccgttaccacctccgtgggaactggcaccagatctctgctcggaagctctggctctctggccgcctatgtgcccctgctgacccagggatgggccgaaatcctggtgcgaaggccaacaggcaacacctcctggctgatgagcctggagaatcctggctccggcatcaacccttctttcgtgttcctgcagctgtaccattcccccttcttcggcgacgagagcaacaagccaatcctgctgcctaacgagtcccagagcttcgagcgatcggtgcagctgctggaccagatccccagttatgacacccataaaatcgccgtgctgtacgtgggagagggccagagcaacagcgagctggccatcctgtcaaatgaacacggcagctacaggtatactgagttcctcacaggcctgggaagactgatcgagctgaaggattgccagcctgataaggtgtacctgggaggcctggacgtgtgcggtgaagacgggcagtttacctactgctggcatgatgatatcatgcaggcagtgttccatatcgccacactgatgcccaccaaggatgtggacaaacaccgctgcgataagaaaaggcacctgggcaacgacttcgtgagtatcgtgtacaacgacagcggggaggacttcaaactgggcaccattaaaggacagttcaattttgtccacgtgatcgtgactcccctggactacgagtgcaacctggtgagtctgcagtgccgcaaagatatggagggcctggtggacacctccgtggctaagatcgtgagcgacagaaacctgcctttcgtggcacggcagatggccctgcacgccaacatggccagccaggtgcaccactcaaggtccaaccccaccgacatctatcccagcaaatggatcgctcgcctgcggcacatcaaacggctcaggcagcggatctgtgaggaggctgcctatagcaaccccagttgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - protein, 966 aa, synthetic construct
MPLEARHAVLALLKAIVQGQGERLGVLRALFFKVIKDYPSNEDLHERLEVFKALTDNGRHITYLEEELADFVLQWMDVGLSSEFLLVLVNLVKFNSGSGCLPAESLPLFIVTLCRTINVKELCEPCWKLMRNLLGTHGSSGSSGSVVAWDILLNIIERLLQQLQTLDSPELRTIVHDLLTTVEELCDQNEFHGSQERYFELVERCADQRPGSSGGAVRIKVLDVLSFVLLINRQFYEEELINSVVISQLSHIPEDKDHQVRKLATQLLVDLAEGCHGSGASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGSSGSLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 22 - DNA, 3090 bp, synthetic construct
  source 1..3090: mol_type = other DNA
atggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccggatcatccggtagctctggcagctctggcagttctggtagcagcggctccgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgggtcaggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctccgggaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggctctagcggcagcctggcggcctatgtgcccctgctgacccagggctgggcggagatcctggtccggaggcccacagggaacaccagctggctgatgagcctggagaacccgggcagcggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 23 - DNA, 3090 bp, synthetic construct
  source 1..3090: mol_type = other DNA
atggaatgcggcctgaataatagaatcaggatgatcggccagatttgcgaggtggcaaagacaaagaagttcgaggagcacgcagtggaggctctgtggaaagccgtggccgacctgctgcagccagagaggcccctggaggccaggcacgccgtgctggccctgctgaaagccatcgtgcagggccagggcgagaggctgggggtgctgagggcccttttcttcaaagtgattaaggactacccttctaacgaggatctgcatgagcggctggaggtgttcaaggccctgaccgacaacggacgccatatcacctacctggaggaggaactggccgacttcgtgctccagtggatggacgtgggactgtcctctgagtttctgctggtgctggtgaatctggtgaagttcaatagctgttacctggatgagtacatcgctagaatggtgcagatgatctgcctgctgtgcgtgagaaccgctagtagcgtcgatatcgaagtgtctctgcaggtgctggacgccgtggtctgctacaattgtctccccgccgagagcctgcctctgttcattgtgacactgtgccggaccatcaatgtgaaagaactgtgtgaaccatgctggaagctgatgagaaatctgctgggcacccacctgggccactctgccatctataacatgtgtcacctgatggaggacagagcatatatggaagacgcccctttgctgcggggcgccgtgttcttcgtgggcatggccctgtggggggctcacaggctgtattcactgcgcaattcacccacctccgtgctgcctagcttttaccaggccatggcctgccccaacgaagtggtgagctacgaaatcgtgctgagcattacgcgcctgattaagaaataccggaaggaactgcaggtggtggcctgggacatcctgctgaacattatcgagcggctgctgcagcagctgcagacaggatcatccggtagctctggcagctctggcagttctggtagcagcggctccgcctcccatgccacacgcgtctacgaaatgctggtgagccacatccagctgcactataagcacagctacacactgcccatcgcttcctccatcagactgcaggccttcgatttcctgctgctgttgcgggctgactcactgcataggctggggctgcccaataaggatggcgtggtgagattcagcccatactgtgtgtgcgggtcaggctcactgctctttcgggtgctgctgcagtgcctgaaacaggagagcgactggaaagtcctgaaactggtgctgggccgcctgcccgagagcctgcgatacaaggtgctgatcttcacatccccttgtagcgtggaccagctgtgcagcgccctgtgtagcatgctgtctggcccaaagaccctcgaacggttgcgcggcgccccagagggcttctcccgcactgacctgcacctggctgtggtgcctgtgttgaccgccctcattagctaccacaattatctcgacaagaccaagcagagagagatggtgtattgcctggagcagggcctgattcaccggtgcgcttctcaatgcgtggtcgccctgagtatttgcagcgtggagatgccagacattattatcaaggccctgcctgtgcttgtggtgaaactgactcacattagcgccacagctagcatggctgtgcctctgttggaattcctgtccacactggccagactgccccacctgtataggaactttgcagccgagcagtacgcctcagtgttcgccatttccctgccttacaccaatccttctaagtttaaccagtatatcgtgtgcctggcccaccacgtgattgccatgtggttcatcaggtgcaggctgcctttccggaaggacttcgtccctttcattaccaagggtctgaggtccaatgtgctgctgtcaggctccgggaagaacctgcacctcgagctgactgaaacctgcctggacatgatggccaggtacgtgtttagtaacttcactgctgtgcccaaacggtcacctgtgggggagtttctgctggccggcgggcgcacaaagacctggctggtggggaataagctggtgaccgtgacaaccagtgtgggcaccggcaccagatctttactgggctctagcggcagcctggccgcctatgtgcccctgctgactcagggctgggccgaaattctggtgagacggcccaccggcaataccagctggctgatgagcctggagaaccctggcagcggcataaaccctagcttcgtgttcctgcagctgtaccacagccctttctttggcgacgagtctaataagcccatcctgctgccaaacgagtcccagagcttcgagcgctctgtgcagctgctggaccagatcccctcttacgatacccacaaaatcgccgtgctgtacgtgggcgaaggccagtctaactccgagctggccatcctgtctaacgagcacggcagttacagatatactgagtttctgacagggctgggccgcctgattgagctcaaagactgccagcccgacaaggtgtacctgggaggactggacgtgtgcggcgaggacggacagttcacatattgctggcacgatgatatcatgcaggccgtgtttcacattgccacattgatgcccaccaaggatgtggacaaacacaggtgcgacaagaagcgccacctgggcaacgacttcgtgtctatcgtgtacaacgattccggggaagattttaaactcgggacaatcaagggccagtttaacttcgtgcacgtgatcgtgaccccactggactacgagtgcaatctggtgtctctgcagtgcagaaaggatatggagggtctggtggataccagcgtcgccaagattgtgtccgatcgcaacctgccattcgtggcccggcagatggccctgcacgccaacatggcctcccaggtgcaccactctagatccaacccaacggacatctatccttccaaatggatcgcccgcctgcgccacattaagcggctgcgccagcgcatttgcgaggaggccgcctactccaatccatcttga

SEQ ID NO: 24 - DNA, 4751 bp, synthetic construct
  source 1..4751: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccggatcatccggtagctctggcagctctggcagttctggtagcagcggctccgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgggtcaggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctccgggaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggctctagcggcagcctggcggcctatgtgcccctgctgacccagggctgggcggagatcctggtccggaggcccacagggaacaccagctggctgatgagcctggagaacccgggcagcggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 4676 bp, synthetic construct
  source 1..4676: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatggaatgcggcctgaataatagaatcaggatgatcggccagatttgcgaggtggcaaagacaaagaagttcgaggagcacgcagtggaggctctgtggaaagccgtggccgacctgctgcagccagagaggcccctggaggccaggcacgccgtgctggccctgctgaaagccatcgtgcagggccagggcgagaggctgggggtgctgagggcccttttcttcaaagtgattaaggactacccttctaacgaggatctgcatgagcggctggaggtgttcaaggccctgaccgacaacggacgccatatcacctacctggaggaggaactggccgacttcgtgctccagtggatggacgtgggactgtcctctgagtttctgctggtgctggtgaatctggtgaagttcaatagctgttacctggatgagtacatcgctagaatggtgcagatgatctgcctgctgtgcgtgagaaccgctagtagcgtcgatatcgaagtgtctctgcaggtgctggacgccgtggtctgctacaattgtctccccgccgagagcctgcctctgttcattgtgacactgtgccggaccatcaatgtgaaagaactgtgtgaaccatgctggaagctgatgagaaatctgctgggcacccacctgggccactctgccatctataacatgtgtcacctgatggaggacagagcatatatggaagacgcccctttgctgcggggcgccgtgttcttcgtgggcatggccctgtggggggctcacaggctgtattcactgcgcaattcacccacctccgtgctgcctagcttttaccaggccatggcctgccccaacgaagtggtgagctacgaaatcgtgctgagcattacgcgcctgattaagaaataccggaaggaactgcaggtggtggcctgggacatcctgctgaacattatcgagcggctgctgcagcagctgcagacaggatcatccggtagctctggcagctctggcagttctggtagcagcggctccgcctcccatgccacacgcgtctacgaaatgctggtgagccacatccagctgcactataagcacagctacacactgcccatcgcttcctccatcagactgcaggccttcgatttcctgctgctgttgcgggctgactcactgcataggctggggctgcccaataaggatggcgtggtgagattcagcccatactgtgtgtgcgggtcaggctcactgctctttcgggtgctgctgcagtgcctgaaacaggagagcgactggaaagtcctgaaactggtgctgggccgcctgcccgagagcctgcgatacaaggtgctgatcttcacatccccttgtagcgtggaccagctgtgcagcgccctgtgtagcatgctgtctggcccaaagaccctcgaacggttgcgcggcgccccagagggcttctcccgcactgacctgcacctggctgtggtgcctgtgttgaccgccctcattagctaccacaattatctcgacaagaccaagcagagagagatggtgtattgcctggagcagggcctgattcaccggtgcgcttctcaatgcgtggtcgccctgagtatttgcagcgtggagatgccagacattattatcaaggccctgcctgtgcttgtggtgaaactgactcacattagcgccacagctagcatggctgtgcctctgttggaattcctgtccacactggccagactgccccacctgtataggaactttgcagccgagcagtacgcctcagtgttcgccatttccctgccttacaccaatccttctaagtttaaccagtatatcgtgtgcctggcccaccacgtgattgccatgtggttcatcaggtgcaggctgcctttccggaaggacttcgtccctttcattaccaagggtctgaggtccaatgtgctgctgtcaggctccgggaagaacctgcacctcgagctgactgaaacctgcctggacatgatggccaggtacgtgtttagtaacttcactgctgtgcccaaacggtcacctgtgggggagtttctgctggccggcgggcgcacaaagacctggctggtggggaataagctggtgaccgtgacaaccagtgtgggcaccggcaccagatctttactgggctctagcggcagcctggccgcctatgtgcccctgctgactcagggctgggccgaaattctggtgagacggcccaccggcaataccagctggctgatgagcctggagaaccctggcagcggcataaaccctagcttcgtgttcctgcagctgtaccacagccctttctttggcgacgagtctaataagcccatcctgctgccaaacgagtcccagagcttcgagcgctctgtgcagctgctggaccagatcccctcttacgatacccacaaaatcgccgtgctgtacgtgggcgaaggccagtctaactccgagctggccatcctgtctaacgagcacggcagttacagatatactgagtttctgacagggctgggccgcctgattgagctcaaagactgccagcccgacaaggtgtacctgggaggactggacgtgtgcggcgaggacggacagttcacatattgctggcacgatgatatcatgcaggccgtgtttcacattgccacattgatgcccaccaaggatgtggacaaacacaggtgcgacaagaagcgccacctgggcaacgacttcgtgtctatcgtgtacaacgattccggggaagattttaaactcgggacaatcaagggccagtttaacttcgtgcacgtgatcgtgaccccactggactacgagtgcaatctggtgtctctgcagtgcagaaaggatatggagggtctggtggataccagcgtcgccaagattgtgtccgatcgcaacctgccattcgtggcccggcagatggccctgcacgccaacatggcctcccaggtgcaccactctagatccaacccaacggacatctatccttccaaatggatcgcccgcctgcgccacattaagcggctgcgccagcgcatttgcgaggaggccgcctactccaatccatcttgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - protein, 1029 aa, synthetic construct
MECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTGSSGSSGSSGSSGSSGSASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGSSGSLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 27 - DNA, 2994 bp, synthetic construct
  source 1..2994: mol_type = other DNA
atgagcgccatttacaacatgtgccacctgatggaggaccgcgcctacatggaagacgctcccctgctgcgcggggccgtgttctttgtgggcatggctctgtggggcgcccacaggctgtattccctgaggaacagccccacttctgtgctgccaagcttttaccaggccatggcctgcccaaatgaggtggtgtcttatgagatcgtgctgagcattaccaggctgatcaagaagtacaggaaggagctgcaggtggtggcctgggatattctgctgaacattatcgaaaggctgctccagcagctgcagaccctggactcacccgagctgaggaccatcgtgcacgatctgctgaccaccgtagaggagctgtgcgatcagaatgagtttcatgggtcccaggaacggtactttgagctggtggagagatgcgccgaccagaggccagagagcagccttctgaacctgatcagctacagagcccagagcatccaccccgccaaggacggctggattcagaacctccaggctctgatggagcggttctttaggtctgaaagcagaggcgccgtgcgcatcaaagtgctggacgtgctgtccttcgtgcttcttattaatcggcagttctatgaggaggagctgatcaacagcgttgtgatctctcagctgtcccatatccctgaggacaaggaccatcaggtgaggaagctcgccacccagctgctggttgacttggccgagggctgccatacccatcactttaatagcctgctggatatcatcgagaaggtgatggcccggtccctcagcccccctccagaactggaggagcgggacgtggccgcctactcagcatccctggaggatgtgaagaccgccgtgctgggcctgctggtcattctgcagaccaaactgtacaccctgcccgccagccatgctacacgggtgtacgagatgctggtgagccacatccagctgcactacaagcacagctacaccctgcctattgccagctccatccgcctgcaggccttcgactttctgctgctcctccgggccgattcactgcaccggctgggtctgcccaacaaggacggcgtggtgagattttctccttactgcgtgtgcggctccggcagcctcctgtttagagtgctgctgcagtgtctcaagcaggaatccgactggaaagtgctgaagctggtgctgggcaggctgcccgaaagcctgaggtacaaggtcctgatcttcacttcaccctgtagcgtggatcagctgtgcagcgccctgtgcagcatgctgagcgggcctaagactctggagagactgagaggcgcccctgagggcttttccagaaccgacctgcacctggccgtggtgccagtgctgaccgccctgatctcctatcacaattatctggacaagaccaagcagagagagatggtgtactgcctggagcagggcctgatccatcggtgtgccagccagtgcgtggtggccctgtccatttgctccgtggaaatgcccgacatcatcatcaaggccctgcccgtgctggtggtgaagctgacccatatctccgccaccgccagcatggccgtgcccctgctggaatttctgtctaccctggctcgtctgccacatctgtaccggaattttgcggccgagcagtacgcatccgtgttcgctatcagcctgccttacacaaatccctccaagttcaatcaatacattgtatgtctggcccaccacgtgatcgctatgtggttcatcaggtgcaggctgcccttcagaaaggatttcgtgccatttatcaccaagggcctgaggagcaatgtactgctgagcggctcaggaaagaacctgcatctggaactgaccgagacctgtctggacatgatggctcgatacgtgttctccaacttcaccgctgtacccaagcggtcaccagtgggggaattcctgctggccgggggcaggactaagacatggctggtcggcaacaaactggttactgtgaccacctccgtcggcactggcactagaagtctgctgggctccagtggcagcctggccgcctacgtgccactgctgacccaggggtgggccgagatcctggtgagacgcccaactggcaacacttcatggctgatgtccctggagaaccctggatccggcatcaatccaagcttcgtgttcctgcagctttaccactcaccatttttcggagacgagtccaacaagcccattctgctgccaaacgaatcccagtctttcgagagaagcgtgcagctgctggaccagattcccagctacgacacccacaaaattgccgtgctctacgtgggcgaaggccagagcaactccgagctggccattctgtctaacgagcacggatcttacagatacaccgagtttctgaccggactggggcgactgatcgagctgaaggactgtcagcccgacaaggtatacctgggaggcctggacgtgtgcggcgaggatggccagtttacctattgctggcatgatgacatcatgcaggccgtgttccacatcgctaccctgatgcctacaaaggacgtggataagcataggtgcgataagaagaggcacctgggcaacgacttcgtgtctatcgtgtacaacgattctggcgaggactttaagctgggtaccattaagggccagtttaatttcgtgcatgtgatcgtgacccctctggattatgagtgtaatctggtgagcctgcagtgcagaaaggatatggagggactggtggacacctctgtcgcaaaaatcgtgagcgacagaaatctgcctttcgtggccaggcagatggccctgcatgccaatatggccagtcaggtgcaccacagccggtctaatcccaccgacatctaccccagtaagtggattgcccgactccggcacatcaagaggctgcgccagcggatttgtgaggaagctgcctatagcaaccctagctga

SEQ ID NO: 28 - DNA, 3464 bp, synthetic construct
  source 1..3464: mol_type = other DNA
atgaagtttgaagagcatgccgtggaagccctgtggaaggccgtggctgacctgctgcagcctgaacgtcctctggaagctcgccatgccgtgttggccctgctgaaggctatcgtgcagggacagggagagaggctgggcgtgctgcgcgccctgttctttaaggtgattaaggactacccctccaacgaggacctgcacgagaggctggaagtgtttaaggccctgaccgataacggcaggcacatcacttacctggaggaggagctggccgacttcgtgctgcagtggatggatgtgggcctgagcagcgaattcctgctggtgctggtgaatctggtgaaattcaacagctgttacctggatgaatacattgccagaatggtccagatgatctgcctgctttgcgtgcgaacagcctcatcagtggatatcgaggtgtctctgcaggtgctggacgccgtggtgtgctataactgtctgcccgctgagagcctccctctgtttatcgtgacactgtgccgcaccatcaacgtgaaagaactgtgcgagccttgctggaaactgatgagaaacctgctggggacacacctgggccacagcgccatctacaacatgtgccacctcatggaagaccgggcctacatggaggatgcccccctgctgcgcggggccgtgtttttcgtgggaatggctctgtggggggcccatagactgtacagcctcaggaattccccaaccagcgtgctgccatccttttatcaggccatggcctgtcccaacgaagtggtgagctatgagatcgtcctgtccatcacccggctgattaaaaaataccggaaagagctgcaggtggtggcttgggatatcctgctgaatatcatcgagaggctgctgcagcagctgcagactctcgacagccctgagctgcgcaccattgtgcacgatttgctgaccaccgtggaagagctgtgcgatcagaacgaattccacggctcccaggagaggtactttgagctggtggaaagatgtgccgaccagaggcctgagtctagtctcctgaatctgatctcctacagagctcaatccatccatggtggaagcggcggcagtggaggctccaccctgcccgccagccacgccaccagagtgtacgagatgctggtcagtcacatccagctgcactacaaacattcctacactctgccaatcgccagctcaatccggctgcaggccttcgactttctgctgctgctgagagccgacagcctgcacagactcggcctgcctaacaaggatggtgtggtgaggttcagtccatactgtgtgtgcgggtctggctctctgctgttcagagtcctgctgcagtgcctcaagcaggagagcgactggaaggtcctgaagctggtcctgggccgtctgcccgagagtctgaggtacaaggtgctgattttcaccagcccctgcagtgtggaccagctgtgttccgccctctgcagcatgctgagcggcccaaagaccctggagcggctgaggggcgcccccgagggatttagcaggaccgatctgcacctggccgtggtgcccgtgctgactgccctgatctcctaccacaattacctggataagaccaaacagcgggagatggtgtattgcctggagcaggggctgatccatcgctgtgcctctcagtgcgtggtggccctgagtatctgcagcgtggaaatgcccgatatcattatcaaggccctgcctgtgctggtggtgaagctgacccacatcagcgccaccgccagcatggccgtgcctctcctggagttcctgagtacactggccagactgccccatctgtatagaaattttgccgcagaacagtacgccagcgtgttcgccatctctctgccatacacaaatccctccaagttcaaccagtatatcgtgtgtctcgcccaccacgtgatcgcaatgtggttcatccgctgccggcttcccttcaggaaggacttcgtgccctttatcactaagggactgaggagtaacgtgctgctcagtggcagcgggaagaacctgcatctcgagctgaccgagacatgcctggacatgatggctaggtatgtgttctccaacttcacagccgtgcccaaacggtccccagtgggggaatttctgctggcagggggcagaaccaagacatggctggtggggaataagctggtgaccgtgaccacctccgtgggcactggcacccggagcctgctgggcgggagcggcggcctggccgcctacgtgcctctgctgacacagggctgggcagagatcctggtgagaagacctacaggcaacaccagctggctgatgtccctggagaaccccggtagtggcattaatccatcttttgtgtttctgcagctgtaccatagccccttcttcggcgatgaaagcaataagcctatcctgctgcccaacgagagccagtcattcgagaggagcgtgcagctgctcgatcagatccctagctatgacactcacaaaatcgccgtgctgtacgtgggcgaggggcagtccaactctgagctggccatcctgagcaatgagcacgggagctacagatataccgagtttctgaccggcctgggccggctgattgagctgaaggattgccagcccgacaaagtgtacctgggcggcctggatgtgtgcggggaggacggccagtttacttactgttggcacgacgatatcatgcaggccgtgttccacatcgctactctgatgcctacaaaagatgtggacaagcaccggtgcgacaaaaaaagacatctgggaaatgactttgtgtccattgtgtacaatgactccggcgaggactttaaactgggaaccatcaagggccagttcaatttcgtgcacgtgatcgtgactccactggattacgagtgtaatctcgtgagcctgcagtgcaggaaagacatggaagggctggtggatacctctgtggccaaaatcgtgtcagacaggaacctgccctttgtggctcggcagatggccctgcacgccaatatggccagtcaggtgcaccactcccggagcaaccctacagatatctatcccagcaaatggatcgcccgcctgaggcacattaagcgcctgcggcagagaatctgtgaagaggccgcctacagcaacccatcatgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgc

SEQ ID NO: 29 - DNA, 4655 bp, synthetic construct
  source 1..4655: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggctccggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggctcaggaaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggctccagtggcagcctggcggcctatgtgcccctgctgacccagggctgggcggagatcctggtccggaggcccacagggaacaccagctggctgatgagcctggagaacccgggatccggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 4655 bp, synthetic construct
  source 1..4655: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgagcgccatttacaacatgtgccacctgatggaggaccgcgcctacatggaagacgctcccctgctgcgcggggccgtgttctttgtgggcatggctctgtggggcgcccacaggctgtattccctgaggaacagccccacttctgtgctgccaagcttttaccaggccatggcctgcccaaatgaggtggtgtcttatgagatcgtgctgagcattaccaggctgatcaagaagtacaggaaggagctgcaggtggtggcctgggatattctgctgaacattatcgaaaggctgctccagcagctgcagaccctggactcacccgagctgaggaccatcgtgcacgatctgctgaccaccgtagaggagctgtgcgatcagaatgagtttcatgggtcccaggaacggtactttgagctggtggagagatgcgccgaccagaggccagagagcagccttctgaacctgatcagctacagagcccagagcatccaccccgccaaggacggctggattcagaacctccaggctctgatggagcggttctttaggtctgaaagcagaggcgccgtgcgcatcaaagtgctggacgtgctgtccttcgtgcttcttattaatcggcagttctatgaggaggagctgatcaacagcgttgtgatctctcagctgtcccatatccctgaggacaaggaccatcaggtgaggaagctcgccacccagctgctggttgacttggccgagggctgccatacccatcactttaatagcctgctggatatcatcgagaaggtgatggcccggtccctcagcccccctccagaactggaggagcgggacgtggccgcctactcagcatccctggaggatgtgaagaccgccgtgctgggcctgctggtcattctgcagaccaaactgtacaccctgcccgccagccatgctacacgggtgtacgagatgctggtgagccacatccagctgcactacaagcacagctacaccctgcctattgccagctccatccgcctgcaggccttcgactttctgctgctcctccgggccgattcactgcaccggctgggtctgcccaacaaggacggcgtggtgagattttctccttactgcgtgtgcggctccggcagcctcctgtttagagtgctgctgcagtgtctcaagcaggaatccgactggaaagtgctgaagctggtgctgggcaggctgcccgaaagcctgaggtacaaggtcctgatcttcacttcaccctgtagcgtggatcagctgtgcagcgccctgtgcagcatgctgagcgggcctaagactctggagagactgagaggcgcccctgagggcttttccagaaccgacctgcacctggccgtggtgccagtgctgaccgccctgatctcctatcacaattatctggacaagaccaagcagagagagatggtgtactgcctggagcagggcctgatccatcggtgtgccagccagtgcgtggtggccctgtccatttgctccgtggaaatgcccgacatcatcatcaaggccctgcccgtgctggtggtgaagctgacccatatctccgccaccgccagcatggccgtgcccctgctggaatttctgtctaccctggctcgtctgccacatctgtaccggaattttgcggccgagcagtacgcatccgtgttcgctatcagcctgccttacacaaatccctccaagttcaatcaatacattgtatgtctggcccaccacgtgatcgctatgtggttcatcaggtgcaggctgcccttcagaaaggatttcgtgccatttatcaccaagggcctgaggagcaatgtactgctgagcggctcaggaaagaacctgcatctggaactgaccgagacctgtctggacatgatggctcgatacgtgttctccaacttcaccgctgtacccaagcggtcaccagtgggggaattcctgctggccgggggcaggactaagacatggctggtcggcaacaaactggttactgtgaccacctccgtcggcactggcactagaagtctgctgggctccagtggcagcctggccgcctacgtgccactgctgacccaggggtgggccgagatcctggtgagacgcccaactggcaacacttcatggctgatgtccctggagaaccctggatccggcatcaatccaagcttcgtgttcctgcagctttaccactcaccatttttcggagacgagtccaacaagcccattctgctgccaaacgaatcccagtctttcgagagaagcgtgcagctgctggaccagattcccagctacgacacccacaaaattgccgtgctctacgtgggcgaaggccagagcaactccgagctggccattctgtctaacgagcacggatcttacagatacaccgagtttctgaccggactggggcgactgatcgagctgaaggactgtcagcccgacaaggtatacctgggaggcctggacgtgtgcggcgaggatggccagtttacctattgctggcatgatgacatcatgcaggccgtgttccacatcgctaccctgatgcctacaaaggacgtggataagcataggtgcgataagaagaggcacctgggcaacgacttcgtgtctatcgtgtacaacgattctggcgaggactttaagctgggtaccattaagggccagtttaatttcgtgcatgtgatcgtgacccctctggattatgagtgtaatctggtgagcctgcagtgcagaaaggatatggagggactggtggacacctctgtcgcaaaaatcgtgagcgacagaaatctgcctttcgtggccaggcagatggccctgcatgccaatatggccagtcaggtgcaccacagccggtctaatcccaccgacatctaccccagtaagtggattgcccgactccggcacatcaagaggctgcgccagcggatttgtgaggaagctgcctatagcaaccctagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - protein, 997 aa, synthetic construct
MSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGSSGSLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 32 - DNA, 3195 bp, synthetic construct
  source 1..3195: mol_type = other DNA
atgaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacggtggaagcggcggcagtggaggctccaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgggtctggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggcagcgggaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcgggagcggcggcctggcggcctatgtgcccctgctgacccagggctgggcggagatcctggtccggaggcccacagggaacaccagctggctgatgagcctggagaacccgggtagtggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 33 - DNA, 3195 bp, synthetic construct
  source 1..3195: mol_type = other DNA
atgaagtttgaagagcatgccgtggaagccctgtggaaggccgtggctgacctgctgcagcctgaacgtcctctggaagctcgccatgccgtgttggccctgctgaaggctatcgtgcagggacagggagagaggctgggcgtgctgcgcgccctgttctttaaggtgattaaggactacccctccaacgaggacctgcacgagaggctggaagtgtttaaggccctgaccgataacggcaggcacatcacttacctggaggaggagctggccgacttcgtgctgcagtggatggatgtgggcctgagcagcgaattcctgctggtgctggtgaatctggtgaaattcaacagctgttacctggatgaatacattgccagaatggtccagatgatctgcctgctttgcgtgcgaacagcctcatcagtggatatcgaggtgtctctgcaggtgctggacgccgtggtgtgctataactgtctgcccgctgagagcctccctctgtttatcgtgacactgtgccgcaccatcaacgtgaaagaactgtgcgagccttgctggaaactgatgagaaacctgctggggacacacctgggccacagcgccatctacaacatgtgccacctcatggaagaccgggcctacatggaggatgcccccctgctgcgcggggccgtgtttttcgtgggaatggctctgtggggggcccatagactgtacagcctcaggaattccccaaccagcgtgctgccatccttttatcaggccatggcctgtcccaacgaagtggtgagctatgagatcgtcctgtccatcacccggctgattaaaaaataccggaaagagctgcaggtggtggcttgggatatcctgctgaatatcatcgagaggctgctgcagcagctgcagactctcgacagccctgagctgcgcaccattgtgcacgatttgctgaccaccgtggaagagctgtgcgatcagaacgaattccacggctcccaggagaggtactttgagctggtggaaagatgtgccgaccagaggcctgagtctagtctcctgaatctgatctcctacagagctcaatccatccatggtggaagcggcggcagtggaggctccaccctgcccgccagccacgccaccagagtgtacgagatgctggtcagtcacatccagctgcactacaaacattcctacactctgccaatcgccagctcaatccggctgcaggccttcgactttctgctgctgctgagagccgacagcctgcacagactcggcctgcctaacaaggatggtgtggtgaggttcagtccatactgtgtgtgcgggtctggctctctgctgttcagagtcctgctgcagtgcctcaagcaggagagcgactggaaggtcctgaagctggtcctgggccgtctgcccgagagtctgaggtacaaggtgctgattttcaccagcccctgcagtgtggaccagctgtgttccgccctctgcagcatgctgagcggcccaaagaccctggagcggctgaggggcgcccccgagggatttagcaggaccgatctgcacctggccgtggtgcccgtgctgactgccctgatctcctaccacaattacctggataagaccaaacagcgggagatggtgtattgcctggagcaggggctgatccatcgctgtgcctctcagtgcgtggtggccctgagtatctgcagcgtggaaatgcccgatatcattatcaaggccctgcctgtgctggtggtgaagctgacccacatcagcgccaccgccagcatggccgtgcctctcctggagttcctgagtacactggccagactgccccatctgtatagaaattttgccgcagaacagtacgccagcgtgttcgccatctctctgccatacacaaatccctccaagttcaaccagtatatcgtgtgtctcgcccaccacgtgatcgcaatgtggttcatccgctgccggcttcccttcaggaaggacttcgtgccctttatcactaagggactgaggagtaacgtgctgctcagtggcagcgggaagaacctgcatctcgagctgaccgagacatgcctggacatgatggctaggtatgtgttctccaacttcacagccgtgcccaaacggtccccagtgggggaatttctgctggcagggggcagaaccaagacatggctggtggggaataagctggtgaccgtgaccacctccgtgggcactggcacccggagcctgctgggcgggagcggcggcctggccgcctacgtgcctctgctgacacagggctgggcagagatcctggtgagaagacctacaggcaacaccagctggctgatgtccctggagaaccccggtagtggcattaatccatcttttgtgtttctgcagctgtaccatagccccttcttcggcgatgaaagcaataagcctatcctgctgcccaacgagagccagtcattcgagaggagcgtgcagctgctcgatcagatccctagctatgacactcacaaaatcgccgtgctgtacgtgggcgaggggcagtccaactctgagctggccatcctgagcaatgagcacgggagctacagatataccgagtttctgaccggcctgggccggctgattgagctgaaggattgccagcccgacaaagtgtacctgggcggcctggatgtgtgcggggaggacggccagtttacttactgttggcacgacgatatcatgcaggccgtgttccacatcgctactctgatgcctacaaaagatgtggacaagcaccggtgcgacaaaaaaagacatctgggaaatgactttgtgtccattgtgtacaatgactccggcgaggactttaaactgggaaccatcaagggccagttcaatttcgtgcacgtgatcgtgactccactggattacgagtgtaatctcgtgagcctgcagtgcaggaaagacatggaagggctggtggatacctctgtggccaaaatcgtgtcagacaggaacctgccctttgtggctcggcagatggccctgcacgccaatatggccagtcaggtgcaccactcccggagcaaccctacagatatctatcccagcaaatggatcgcccgcctgaggcacattaagcgcctgcggcagagaatctgtgaagaggccgcctacagcaacccatcatga

SEQ ID NO: 34 - DNA, 4856 bp, synthetic construct
  source 1..4856: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacggtggaagcggcggcagtggaggctccaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcgggtctggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctggcagcgggaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcgggagcggcggcctggcggcctatgtgcccctgctgacccagggctgggcggagatcctggtccggaggcccacagggaacaccagctggctgatgagcctggagaacccgggtagtggcatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 4631 bp, synthetic construct
  source 1..4631: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtccggaattcccgggatatcgtcgacccacgcgtccgggccccacgctgcgcacccgcgggtttgctcaagtttgtacaaaaaagcaggctgccaccatgaagtttgaagagcatgccgtggaagccctgtggaaggccgtggctgacctgctgcagcctgaacgtcctctggaagctcgccatgccgtgttggccctgctgaaggctatcgtgcagggacagggagagaggctgggcgtgctgcgcgccctgttctttaaggtgattaaggactacccctccaacgaggacctgcacgagaggctggaagtgtttaaggccctgaccgataacggcaggcacatcacttacctggaggaggagctggccgacttcgtgctgcagtggatggatgtgggcctgagcagcgaattcctgctggtgctggtgaatctggtgaaattcaacagctgttacctggatgaatacattgccagaatggtccagatgatctgcctgctttgcgtgcgaacagcctcatcagtggatatcgaggtgtctctgcaggtgctggacgccgtggtgtgctataactgtctgcccgctgagagcctccctctgtttatcgtgacactgtgccgcaccatcaacgtgaaagaactgtgcgagccttgctggaaactgatgagaaacctgctggggacacacctgggccacagcgccatctacaacatgtgccacctcatggaagaccgggcctacatggaggatgcccccctgctgcgcggggccgtgtttttcgtgggaatggctctgtggggggcccatagactgtacagcctcaggaattccccaaccagcgtgctgccatccttttatcaggccatggcctgtcccaacgaagtggtgagctatgagatcgtcctgtccatcacccggctgattaaaaaataccggaaagagctgcaggtggtggcttgggatatcctgctgaatatcatcgagaggctgctgcagcagctgcagactctcgacagccctgagctgcgcaccattgtgcacgatttgctgaccaccgtggaagagctgtgcgatcagaacgaattccacggctcccaggagaggtactttgagctggtggaaagatgtgccgaccagaggcctgagtctagtctcctgaatctgatctcctacagagctcaatccatccatggtggaagcggcggcagtggaggctccaccctgcccgccagccacgccaccagagtgtacgagatgctggtcagtcacatccagctgcactacaaacattcctacactctgccaatcgccagctcaatccggctgcaggccttcgactttctgctgctgctgagagccgacagcctgcacagactcggcctgcctaacaaggatggtgtggtgaggttcagtccatactgtgtgtgcgggtctggctctctgctgttcagagtcctgctgcagtgcctcaagcaggagagcgactggaaggtcctgaagctggtcctgggccgtctgcccgagagtctgaggtacaaggtgctgattttcaccagcccctgcagtgtggaccagctgtgttccgccctctgcagcatgctgagcggcccaaagaccctggagcggctgaggggcgcccccgagggatttagcaggaccgatctgcacctggccgtggtgcccgtgctgactgccctgatctcctaccacaattacctggataagaccaaacagcgggagatggtgtattgcctggagcaggggctgatccatcgctgtgcctctcagtgcgtggtggccctgagtatctgcagcgtggaaatgcccgatatcattatcaaggccctgcctgtgctggtggtgaagctgacccacatcagcgccaccgccagcatggccgtgcctctcctggagttcctgagtacactggccagactgccccatctgtatagaaattttgccgcagaacagtacgccagcgtgttcgccatctctctgccatacacaaatccctccaagttcaaccagtatatcgtgtgtctcgcccaccacgtgatcgcaatgtggttcatccgctgccggcttcccttcaggaaggacttcgtgccctttatcactaagggactgaggagtaacgtgctgctcagtggcagcgggaagaacctgcatctcgagctgaccgagacatgcctggacatgatggctaggtatgtgttctccaacttcacagccgtgcccaaacggtccccagtgggggaatttctgctggcagggggcagaaccaagacatggctggtggggaataagctggtgaccgtgaccacctccgtgggcactggcacccggagcctgctgggcgggagcggcggcctggccgcctacgtgcctctgctgacacagggctgggcagagatcctggtgagaagacctacaggcaacaccagctggctgatgtccctggagaaccccggtagtggcattaatccatcttttgtgtttctgcagctgtaccatagccccttcttcggcgatgaaagcaataagcctatcctgctgcccaacgagagccagtcattcgagaggagcgtgcagctgctcgatcagatccctagctatgacactcacaaaatcgccgtgctgtacgtgggcgaggggcagtccaactctgagctggccatcctgagcaatgagcacgggagctacagatataccgagtttctgaccggcctgggccggctgattgagctgaaggattgccagcccgacaaagtgtacctgggcggcctggatgtgtgcggggaggacggccagtttacttactgttggcacgacgatatcatgcaggccgtgttccacatcgctactctgatgcctacaaaagatgtggacaagcaccggtgcgacaaaaaaagacatctgggaaatgactttgtgtccattgtgtacaatgactccggcgaggactttaaactgggaaccatcaagggccagttcaatttcgtgcacgtgatcgtgactccactggattacgagtgtaatctcgtgagcctgcagtgcaggaaagacatggaagggctggtggatacctctgtggccaaaatcgtgtcagacaggaacctgccctttgtggctcggcagatggccctgcacgccaatatggccagtcaggtgcaccactcccggagcaaccctacagatatctatcccagcaaatggatcgcccgcctgaggcacattaagcgcctgcggcagagaatctgtgaagaggccgcctacagcaacccatcatgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - protein, 1064 aa, synthetic construct
MKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHGGSGGSGGSTLPASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGGSGGLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 37 - DNA, 3801 bp, synthetic construct
  source 1..3801: mol_type = other DNA
atggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagcggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctgggagcggcaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcggcagcggaggcctggcggcctatgtgcccctgctgacccagggctgggcggagatcctggtccggaggcccacagggaacaccagctggctgatgagcctggagaacccgggaagtggtatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctga

SEQ ID NO: 38 - DNA, 3801 bp, synthetic construct
  source 1..3801: mol_type = other DNA
atggctaagccaacttctaaggattctggcctgaaggagaagttcaagatcctgctggggctcggcactccacggcctaatcctagatctgccgagggaaagcagaccgagtttatcatcaccgccgaaatcctgagagagctgagcatggaatgcgggctcaataacaggatccggatgatcggccagatttgcgaggtggctaagaccaagaaatttgaagagcatgccgtggaggccctctggaaagccgtggccgacctgttacagcctgagagacccttggaggcccggcacgcagtgctggctctgctgaaagcaatcgtgcagggacagggggagaggctgggcgtgctgagagccctgttcttcaaggtgatcaaggattacccaagcaatgaggatctgcatgaaagactggaggtgtttaaggccctgactgacaacgggcgacacatcacatatctggaggaggagctggccgacttcgtgctgcagtggatggatgtgggcctgtcctccgagttcctgctggtgctggtcaatctggtgaagttcaatagctgctatctggacgaatacatcgcccggatggtgcagatgatctgcctgctgtgtgtgaggacagccagcagcgtggacatcgaggtgtcactccaggtgctggatgccgtggtgtgttacaactgtctccccgccgagagcctgcccctgtttatcgtcaccctgtgcagaaccattaacgtgaaggaactgtgtgagccatgttggaagctgatgcggaacctgctgggcacccacctgggccattctgccatctacaatatgtgccacctgatggaggatcgggcctatatggaggacgcccctctgctgagaggcgccgtgtttttcgtcggcatggcactctggggcgcccaccgactgtatagcctccgcaactcccctaccagcgtgctgccctctttctatcaggccatggcctgccccaatgaggtcgtgtcttacgagatcgtgctcagcatcaccaggctgattaagaagtacaggaaggagctgcaggtggtggcctgggatattctgttaaacatcatcgagcgcctgctgcagcagctgcagactctggactcccctgagctgcggaccattgtccacgatctgctgaccaccgtggaggagctgtgcgatcagaacgaattccacggctctcaggagcggtattttgagctggtggagcggtgcgccgaccagagacctgagtcatcactgctgaacctgatctcctacagggctcagtccatccacccagccaaagacggctggatccagaacctgcaggcactgatggagcgcttcttccggtctgaatctcggggcgccgtaaggatcaaggtgctggatgtgctgagcttcgtgctgctgatcaaccggcagttctacgaggaggagctgatcaactctgtggtgatcagtcagctgtctcatatccccgaggataaagaccaccaggtgagaaagctggccacccagctgctcgtggatctggccgagggctgccacacacaccacttcaacagcctgctggacatcatcgaaaaagtgatggcccgttccctgagccccccacccgagctggaagagcgcgatgtggccgcttattctgccagcctggaagacgtgaaaactgccgtcctgggcctgctggtgattctgcagaccaagctgtatacattgccagccagtcacgccaccagggtgtacgagatgctggtgagccatatccagctccactataagcactcttataccctgccaattgccagctccattaggctgcaggcctttgacttcctgctgctgctgagggctgacagcctgcacagactgggactgcctaacaaggatggggtggtgcgcttcagcccctactgcgtgtgcggcagcggctctctgctgtttagggtgctgctgcagtgtctcaaacaggagagcgactggaaagtgctgaaactggtgctgggccgcctgcccgaatccctgaggtacaaagtgctgatcttcacatccccatgctccgtggaccagctgtgttcagccctgtgctcaatgctgtccggacccaaaacactggagagactgaggggcgccccagagggcttttccaggacagacctgcacctggcagtggtgcctgtgttgaccgcactgatctcctatcacaactatctggataagaccaagcagagggagatggtgtactgcctggagcagggactgattcaccgttgcgccagccagtgcgtggtcgctctgtcaatctgcagtgtggagatgcccgatatcatcatcaaggctctgcctgtcctggtggtgaagctgacacacatcagcgccaccgccagcatggctgtgcccctgctggagtttctgagcactctggcccggctgccccacctgtaccgtaactttgccgccgagcagtacgcctccgtgtttgccattagcttgccctacaccaacccatccaagttcaatcaatatatcgtgtgcctggcccaccacgtgatcgctatgtggttcatcagatgccgcctccccttccggaaggatttcgtgccctttatcaccaaagggctgaggtctaacgtgctgctctctgggagcggcaagaatctgcacctggaactgaccgagacctgcctggacatgatggccaggtacgtgttttccaactttacagccgtgcctaaacgctccccagtcggcgaatttctgctggccgggggccgcactaagacctggctcgtgggcaacaagctcgtgaccgtgaccaccagcgtgggtactgggacacggtccctgctgggcggcagcggaggcctggccgcctacgtgcctctgcttacacagggctgggccgaaatcctggtgcgacgcccaacgggaaacacctcttggctgatgtccctggagaatcctggaagtggtatcaatcctagcttcgtgtttctgcagctgtaccacagcccttttttcggcgacgagtccaataaacccatcctgctgcctaacgagtcccagagcttcgagcggtcagtgcagcttctggatcagatcccaagctacgacacacacaagatcgctgtgctgtatgttggcgaaggccagagtaatagcgagctggccatcctgagcaatgagcacggcagctaccgctatacagagttcctgaccggcctcggaaggctgattgagctgaaggattgtcagccagataaggtctatctgggcggcctggatgtgtgcggcgaggatggccagtttacatattgctggcacgatgatatcatgcaggccgtgttccacatcgccacactgatgcctaccaaagatgtggacaagcacaggtgcgacaagaaaagacacctgggcaacgacttcgtgtccatcgtgtacaatgacagtggagaggacttcaagctgggcaccattaagggccagttcaatttcgtgcacgttattgttacacctctggattacgaatgcaacctcgtgagtctgcagtgtaggaaggacatggagggcttggtggacacctctgtggccaagattgtgagcgacagaaatctgccctttgtggccaggcagatggcactgcatgccaatatggcctctcaggtgcaccactcccggtcaaatcccactgacatctatccctccaagtggatcgccagactgagacatattaagcgcctgcgccagcgaatctgtgaggaggccgcctattccaacccatcatga

SEQ ID NO: 39 - DNA, 4921 bp, synthetic construct
  source 1..4921: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtttagtctttttgtcttttatttcaggtcccggatccggtggtggtgcaaatcaaagaactgctcctcagtggatgttgcctttacttctagcaagtttgtacaaaaaagcaggctgccaccatggccaaaccaacaagcaaagattcaggcttgaaggagaagtttaagattctgttgggactgggaacaccgaggccaaatcccaggtctgcagagggtaaacagacggagtttatcatcaccgcggaaatactgagagaactgagcatggaatgtggcctcaacaatcgcatccggatgatagggcagatttgtgaagtcgcaaaaaccaagaaatttgaagagcacgcagtggaagcactctggaaggcggtcgcggatctgttgcagccggagcggccgctggaggcccggcacgcggtgctggctctgctgaaggccatcgtgcaggggcagggcgagcgtttgggggtcctcagagccctcttctttaaggtcatcaaggattacccttccaacgaagaccttcacgaaaggctggaggttttcaaggccctcacagacaatgggagacacatcacctacttggaggaagagctggctgactttgtcctgcagtggatggatgttggcttgtcctcggaattccttctggtgctggtgaacttggtcaaattcaatagctgttacctcgacgagtacatcgcaaggatggttcagatgatctgtctgctgtgcgtccggaccgcgtcctctgtggacatagaggtctccctgcaggtgctggacgccgtggtctgctacaactgcctgccggctgagagcctcccgctgttcatcgttaccctctgtcgcaccatcaacgtcaaggagctctgcgagccttgctggaagctgatgcggaacctccttggcacccacctgggccacagcgccatctacaacatgtgccacctcatggaggacagagcctacatggaggacgcgcccctgctgagaggagccgtgttttttgtgggcatggctctctggggagcccaccggctctattctctcaggaactcgccgacatctgtgttgccatcattttaccaggccatggcatgtccgaacgaggtggtgtcctatgagatcgtcctgtccatcaccaggctcatcaagaagtataggaaggagctccaggtggtggcgtgggacattctgctgaacatcatcgaacggctccttcagcagctccagaccttggacagcccggagctcaggaccatcgtccatgacctgttgaccacggtggaggagctgtgtgaccagaacgagttccacgggtctcaggagagatactttgaactggtggagagatgtgcggaccagaggcctgagtcctccctcctgaacctgatctcctatagagcgcagtccatccacccggccaaggacggctggattcagaacctgcaggcgctgatggagagattcttcaggagcgagtcccgaggcgccgtgcgcatcaaggtgctggacgtgctgtcctttgtgctgctcatcaacaggcagttctatgaggaggagctgattaactcagtggtcatctcgcagctctcccacatccccgaggataaagaccaccaggtccgaaagctggccacccagttgctggtggacctggcagagggctgccacacacaccacttcaacagcctgctggacatcatcgagaaggtgatggcccgctccctctccccacccccggagctggaagaaagggatgtggccgcatactcggcctccttggaggatgtgaagacagccgtcctggggcttctggtcatccttcagaccaagctgtacaccctgcctgcaagccacgccacgcgtgtgtatgagatgctggtcagccacattcagctccactacaagcacagctacaccctgccaatcgcgagcagcatccggctgcaggcctttgacttcctgttgctgctgcgggccgactcactgcaccgcctgggcctgcccaacaaggatggagtcgtgcggttcagcccctactgcgtctgcggcagcggctccctgctcttccgcgtcctgctgcagtgcttgaagcaggagtctgactggaaggtgctgaagctggttctgggcaggctgcctgagtccctgcgctataaagtgctcatctttacttccccttgcagtgtggaccagctgtgctctgctctctgctccatgctttcaggcccaaagacactggagcggctccgaggcgccccagaaggcttctccagaactgacttgcacctggccgtggttccagtgctgacagcattaatctcttaccataactacctggacaaaaccaaacagcgcgagatggtctactgcctggagcagggcctcatccaccgctgtgccagccagtgcgtcgtggccttgtccatctgcagcgtggagatgcctgacatcatcatcaaggcgctgcctgttctggtggtgaagctcacgcacatctcagccacagccagcatggccgtcccactgctggagttcctgtccactctggccaggctgccgcacctctacaggaactttgccgcggagcagtatgccagtgtgttcgccatctccctgccgtacaccaacccctccaagtttaatcagtacatcgtgtgtctggcccatcacgtcatagccatgtggttcatcaggtgccgcctgcccttccggaaggattttgtccctttcatcactaagggcctgcggtccaatgtcctcttgtctgggagcggcaaaaacctccacctggagctcacggaaacctgtctggacatgatggctcgatacgtcttctccaacttcacggctgtcccgaagaggtctcctgtgggcgagttcctcctagcgggtggcaggaccaaaacctggctggttgggaacaagcttgtcactgtgacgacaagcgtgggaaccgggacccggtcgttactaggcggcagcggaggcctggcggcctatgtgcccctgctgacccagggctgggcggagatcctggtccggaggcccacagggaacaccagctggctgatgagcctggagaacccgggaagtggtatcaaccccagtttcgtgttcctgcagctctaccattcccccttctttggcgacgagtcaaacaagccaatcctgctgcccaatgagtcacagtcctttgagcggtcggtgcagctcctcgaccagatcccatcatacgacacccacaagatcgccgtcctgtatgttggagaaggccagagcaacagcgagctcgccatcctgtccaatgagcatggctcctacaggtacacggagttcctgacgggcctgggccggctcatcgagctgaaggactgccagccggacaaggtgtacctgggaggcctggacgtgtgtggtgaggacggccagttcacctactgctggcacgatgacatcatgcaagccgtcttccacatcgccaccctgatgcccaccaaggacgtggacaagcaccgctgcgacaagaagcgccacctgggcaacgactttgtgtccattgtctacaatgactccggtgaggacttcaagcttggcaccatcaagggccagttcaactttgtccacgtgatcgtcaccccgctggactacgagtgcaacctggtgtccctgcagtgcaggaaagacatggagggccttgtggacaccagcgtggccaagatcgtgtctgaccgcaacctgcccttcgtggcccgccagatggccctgcacgcaaatatggcctcacaggtgcatcatagccgctccaaccccaccgatatctacccctccaagtggattgcccggctccgccacatcaagcggctccgccagcggatctgcgaggaagccgcctactccaaccccagctgaggccggggccctccctcctgcactggccttggacggtattgcctgtcagtgaaataaataaagtcctgaccccagtgcacagacatagaggcacagattgcagtcagacagctcttttattgactttgtctgcttggtgcgggggttgggggggtgtcgaggctctagaagcggccatgcccacagaagtggtacacagaagcaggcacagccagctccgagggcacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 4696 bp, synthetic construct
  source 1..4696: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttggggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtttagtctttttgtcttttatttcaggtcccggatccggtggtggtgcaaatcaaagaactgctcctcagtggatgttgcctttacttctagcaagtttgtacaaaaaagcaggctgccaccatggctaagccaacttctaaggattctggcctgaaggagaagttcaagatcctgctggggctcggcactccacggcctaatcctagatctgccgagggaaagcagaccgagtttatcatcaccgccgaaatcctgagagagctgagcatggaatgcgggctcaataacaggatccggatgatcggccagatttgcgaggtggctaagaccaagaaatttgaagagcatgccgtggaggccctctggaaagccgtggccgacctgttacagcctgagagacccttggaggcccggcacgcagtgctggctctgctgaaagcaatcgtgcagggacagggggagaggctgggcgtgctgagagccctgttcttcaaggtgatcaaggattacccaagcaatgaggatctgcatgaaagactggaggtgtttaaggccctgactgacaacgggcgacacatcacatatctggaggaggagctggccgacttcgtgctgcagtggatggatgtgggcctgtcctccgagttcctgctggtgctggtcaatctggtgaagttcaatagctgctatctggacgaatacatcgcccggatggtgcagatgatctgcctgctgtgtgtgaggacagccagcagcgtggacatcgaggtgtcactccaggtgctggatgccgtggtgtgttacaactgtctccccgccgagagcctgcccctgtttatcgtcaccctgtgcagaaccattaacgtgaaggaactgtgtgagccatgttggaagctgatgcggaacctgctgggcacccacctgggccattctgccatctacaatatgtgccacctgatggaggatcgggcctatatggaggacgcccctctgctgagaggcgccgtgtttttcgtcggcatggcactctggggcgcccaccgactgtatagcctccgcaactcccctaccagcgtgctgccctctttctatcaggccatggcctgccccaatgaggtcgtgtcttacgagatcgtgctcagcatcaccaggctgattaagaagtacaggaaggagctgcaggtggtggcctgggatattctgttaaacatcatcgagcgcctgctgcagcagctgcagactctggactcccctgagctgcggaccattgtccacgatctgctgaccaccgtggaggagctgtgcgatcagaacgaattccacggctctcaggagcggtattttgagctggtggagcggtgcgccgaccagagacctgagtcatcactgctgaacctgatctcctacagggctcagtccatccacccagccaaagacggctggatccagaacctgcaggcactgatggagcgcttcttccggtctgaatctcggggcgccgtaaggatcaaggtgctggatgtgctgagcttcgtgctgctgatcaaccggcagttctacgaggaggagctgatcaactctgtggtgatcagtcagctgtctcatatccccgaggataaagaccaccaggtgagaaagctggccacccagctgctcgtggatctggccgagggctgccacacacaccacttcaacagcctgctggacatcatcgaaaaagtgatggcccgttccctgagccccccacccgagctggaagagcgcgatgtggccgcttattctgccagcctggaagacgtgaaaactgccgtcctgggcctgctggtgattctgcagaccaagctgtatacattgccagccagtcacgccaccagggtgtacgagatgctggtgagccatatccagctccactataagcactcttataccctgccaattgccagctccattaggctgcaggcctttgacttcctgctgctgctgagggctgacagcctgcacagactgggactgcctaacaaggatggggtggtgcgcttcagcccctactgcgtgtgcggcagcggctctctgctgtttagggtgctgctgcagtgtctcaaacaggagagcgactggaaagtgctgaaactggtgctgggccgcctgcccgaatccctgaggtacaaagtgctgatcttcacatccccatgctccgtggaccagctgtgttcagccctgtgctcaatgctgtccggacccaaaacactggagagactgaggggcgccccagagggcttttccaggacagacctgcacctggcagtggtgcctgtgttgaccgcactgatctcctatcacaactatctggataagaccaagcagagggagatggtgtactgcctggagcagggactgattcaccgttgcgccagccagtgcgtggtcgctctgtcaatctgcagtgtggagatgcccgatatcatcatcaaggctctgcctgtcctggtggtgaagctgacacacatcagcgccaccgccagcatggctgtgcccctgctggagtttctgagcactctggcccggctgccccacctgtaccgtaactttgccgccgagcagtacgcctccgtgtttgccattagcttgccctacaccaacccatccaagttcaatcaatatatcgtgtgcctggcccaccacgtgatcgctatgtggttcatcagatgccgcctccccttccggaaggatttcgtgccctttatcaccaaagggctgaggtctaacgtgctgctctctgggagcggcaagaatctgcacctggaactgaccgagacctgcctggacatgatggccaggtacgtgttttccaactttacagccgtgcctaaacgctccccagtcggcgaatttctgctggccgggggccgcactaagacctggctcgtgggcaacaagctcgtgaccgtgaccaccagcgtgggtactgggacacggtccctgctgggcggcagcggaggcctggccgcctacgtgcctctgcttacacagggctgggccgaaatcctggtgcgacgcccaacgggaaacacctcttggctgatgtccctggagaatcctggaagtggtatcaatcctagcttcgtgtttctgcagctgtaccacagcccttttttcggcgacgagtccaataaacccatcctgctgcctaacgagtcccagagcttcgagcggtcagtgcagcttctggatcagatcccaagctacgacacacacaagatcgctgtgctgtatgttggcgaaggccagagtaatagcgagctggccatcctgagcaatgagcacggcagctaccgctatacagagttcctgaccggcctcggaaggctgattgagctgaaggattgtcagccagataaggtctatctgggcggcctggatgtgtgcggcgaggatggccagtttacatattgctggcacgatgatatcatgcaggccgtgttccacatcgccacactgatgcctaccaaagatgtggacaagcacaggtgcgacaagaaaagacacctgggcaacgacttcgtgtccatcgtgtacaatgacagtggagaggacttcaagctgggcaccattaagggccagttcaatttcgtgcacgttattgttacacctctggattacgaatgcaacctcgtgagtctgcagtgtaggaaggacatggagggcttggtggacacctctgtggccaagattgtgagcgacagaaatctgccctttgtggccaggcagatggcactgcatgccaatatggcctctcaggtgcaccactcccggtcaaatcccactgacatctatccctccaagtggatcgccagactgagacatattaagcgcctgcgccagcgaatctgtgaggaggccgcctattccaacccatcatgaacccagctttcttgtacaaagtgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - protein, 1266 aa, synthetic construct
MAKPTSKDSGLKEKFKILLGLGTPRPNPRSAEGKQTEFIITAEILRELSMECGLNNRIRMIGQICEVAKTKKFEEHAVEALWKAVADLLQPERPLEARHAVLALLKAIVQGQGERLGVLRALFFKVIKDYPSNEDLHERLEVFKALTDNGRHITYLEEELADFVLQWMDVGLSSEFLLVLVNLVKFNSCYLDEYIARMVQMICLLCVRTASSVDIEVSLQVLDAVVCYNCLPAESLPLFIVTLCRTINVKELCEPCWKLMRNLLGTHLGHSAIYNMCHLMEDRAYMEDAPLLRGAVFFVGMALWGAHRLYSLRNSPTSVLPSFYQAMACPNEVVSYEIVLSITRLIKKYRKELQVVAWDILLNIIERLLQQLQTLDSPELRTIVHDLLTTVEELCDQNEFHGSQERYFELVERCADQRPESSLLNLISYRAQSIHPAKDGWIQNLQALMERFFRSESRGAVRIKVLDVLSFVLLINRQFYEEELINSVVISQLSHIPEDKDHQVRKLATQLLVDLAEGCHTHHFNSLLDIIEKVMARSLSPPPELEERDVAAYSASLEDVKTAVLGLLVILQTKLYTLPASHATRVYEMLVSHIQLHYKHSYTLPIASSIRLQAFDFLLLLRADSLHRLGLPNKDGVVRFSPYCVCGSGSLLFRVLLQCLKQESDWKVLKLVLGRLPESLRYKVLIFTSPCSVDQLCSALCSMLSGPKTLERLRGAPEGFSRTDLHLAVVPVLTALISYHNYLDKTKQREMVYCLEQGLIHRCASQCVVALSICSVEMPDIIIKALPVLVVKLTHISATASMAVPLLEFLSTLARLPHLYRNFAAEQYASVFAISLPYTNPSKFNQYIVCLAHHVIAMWFIRCRLPFRKDFVPFITKGLRSNVLLSGSGKNLHLELTETCLDMMARYVFSNFTAVPKRSPVGEFLLAGGRTKTWLVGNKLVTVTTSVGTGTRSLLGGSGGLAAYVPLLTQGWAEILVRRPTGNTSWLMSLENPGSGINPSFVFLQLYHSPFFGDESNKPILLPNESQSFERSVQLLDQIPSYDTHKIAVLYVGEGQSNSELAILSNEHGSYRYTEFLTGLGRLIELKDCQPDKVYLGGLDVCGEDGQFTYCWHDDIMQAVFHIATLMPTKDVDKHRCDKKRHLGNDFVSIVYNDSGEDFKLGTIKGQFNFVHVIVTPLDYECNLVSLQCRKDMEGLVDTSVAKIVSDRNLPFVARQMALHANMASQVHHSRSNPTDIYPSKWIARLRHIKRLRQRICEEAAYSNPS

SEQ ID NO: 42 - protein, 12 aa, synthetic construct
GSSGSSGSSGSS

SEQ ID NO: 43 - skipped

SEQ ID NO: 44 - protein, 5 aa, synthetic construct
GGSGG

SEQ ID NO: 45 - protein, 17 aa, synthetic construct
GSSGSSGSSGSSGSSGS

SEQ ID NO: 46 - protein, 5 aa, synthetic construct
GSSGS

SEQ ID NO: 47 - protein, 8 aa, synthetic construct
GSSGSSGS

SEQ ID NO: 48 - protein, 4 aa, synthetic construct
GSSG

SEQ ID NO: 49 - protein, 9 aa, synthetic construct
GGSGGSGGS

SEQ ID NO: 50 - DNA, 36 bp, synthetic construct
  source 1..36: mol_type = other DNA
ggcagttcgggcagctccgggtcctccggctcctct

SEQ ID NO: 51 - skipped

SEQ ID NO: 52 - DNA, 15 bp, synthetic construct
  source 1..15: mol_type = other DNA
ggcggcagcggaggg

SEQ ID NO: 53 - DNA, 51 bp, synthetic construct
  source 1..51: mol_type = other DNA
ggctcctctggtagctctggaagttccggctccagcggctcctccggatct

SEQ ID NO: 54 - DNA, 15 bp, synthetic construct
  source 1..15: mol_type = other DNA
ggctccagtggcagc

SEQ ID NO: 55 - DNA, 24 bp, synthetic construct
  source 1..24: mol_type = other DNA
ggctcctccggctctagcggcagc

SEQ ID NO: 56 - DNA, 12 bp, synthetic construct
  source 1..12: mol_type = other DNA
ggcagctccgga

SEQ ID NO: 57 - DNA, 27 bp, synthetic construct
  source 1..27: mol_type = other DNA
ggtggaagcggcggcagtggaggctcc

SEQ ID NO: 58 - protein, 6 aa, synthetic construct
HHHHHH

SEQ ID NO: 59 - DNA, 19 bp, synthetic construct
  source 1..19: mol_type = other DNA
gattacaggcctgagccac

SEQ ID NO: 60 - DNA, 19 bp, synthetic construct
  source 1..19: mol_type = other DNA
ccttctgccctggtcatgc

(19) United States
(12) Patent Application Publication
(10) Pub. No.: US 2026/0317386 A1
(43) Pub. Date: Oct. 15, 2026

(54) PROTEIN ENGINEERING MICRO-TUBERIN GENE THERAPY CANDIDATES FOR TUBEROUS SCLEROSIS COMPLEX TYPE 2

(71) Applicants: RESEARCH INSTITUTE AT NATIONWIDE CHILDREN'S HOSPITAL, Columbus, OH (US); OHIO STATE INNOVATION FOUNDATION, Columbus, OH (US)

(72) Inventors: Mark Hester, Columbus, OH (US); Craig McElroy, Columbus, OH (US)

(21) Appl. No.: 19/483,439

(22) PCT Filed: May 16, 2024
(86) PCT No.: PCT/US2024/029763

§ 371 Date: Nov. 11, 2025

Related U.S. Application Data
(60) Provisional application No. 63/467,252, filed on May 17, 2023.

(51) Int. Cl. C07K 14/47; A61K 38/17; A61K 48/00; C12N 15/86

(52) U.S. Cl. CPC C07K 14/4703; A61K 38/1709; A61K 48/0033; A61K 48/005; C07K 14/4706; C12N 15/86; C12N 2750/14143

(57) ABSTRACT

This disclosure is directed to engineered micro-Tuberin constructs that can be used in gene therapy for treating tuberin deficiency/mTOR hyperactivation. The disclosed engineered micro-Tuberins are smaller than wild type Tuberin and retain all important domains and functions of the wild type Tuberin protein. Also disclosed are methods for delivering the engineered micro-Tuberins and methods for treating Tuberin deficiency/mTOR hyperactivation.

### CROSS-REFERENCE TO RELATED PATENT APPLICATION

This application claims the benefit under 35 U.S.C. § 119(e) of U.S. Provisional Application No. 63/467,252, filed May 17, 2023, the entire contents of which are incorporated herein by reference.

### BACKGROUND

Tuberous Sclerosis complex (TSC) is an autosomal dominant disorder caused by mutations in either the TSC1 or TSC2 genes, which affects nearly 1 in every 5,500 newborns, ~80,000 people in the U.S., and 2 million people worldwide. There is currently no cure. TSC1 and TSC2 encode for Hamartin and Tuberin, respectively, and together with TBC1D7, form a protein complex to negatively regulate mammalian target of rapamycin complex 1 (mTORC1) signaling (FIG. 1). Dysfunction of the TSC protein complex results in constitutively active mTORC1 signaling, leading to the formation of non-malignant tumors throughout multiple organs, predominantly in the brain, kidney, lungs, eyes, and heart. Though TSC affects multiple organ systems, the most frequent causes of mortality and morbidity are due to neurologic complications. Such complications include refractory epilepsy, autism, neuropsychiatric issues, and uncontrolled ependymal growth lesions resulting in life-threatening hydrocephalus. The frequency and severity of neuropathology can vary widely among patients, although it is known that mutations in TSC2 are approximately six times more prevalent than in TSC1, and patients with TSC2 mutations show a greater severity of neuropathological symptoms. As epilepsy occurs in approximately 85 percent of patients, the inability to control seizures is one of the most debilitating aspects of the disease as most existing medications show loss of efficacy within 1-2 years of treatment. Other non-neurological and life-threatening complications in TSC include lymphangioleiomyomatosis (LAM), which is characterized by overgrowth of smooth muscle in the lungs leading to respiratory distress, and renal angiomyolipoma (AML), which can result in retroperitoneal bleeding.

There are multiple treatment options available for TSC patients to manage symptoms with variable and limited efficacies that are primarily administered to either control epileptic seizures and/or tumor growth. For TSC patients <1 year of age, anti-seizure treatments are commonly administered to control infantile spasms, which peak between four to six months of age, and are one of the earliest neurological symptoms that manifest in ~40% of TSC patients. Although vigabatrin is one of the most potent antiseizure medications to treat this condition, it is only effective in ~50% of TSC patients, carries the serious side-effect of permanent visual field constriction, and usually loses its efficacy within one year of treatment.

For TSC patients >1 year of age, current treatment options include administration of rapalog mTOR inhibitors, although these drugs cause side effects associated with immune suppression, have variable efficacies in patients, and must be administered continuously for lasting effects. In addition, long-term rapalog treatment in children may adversely affect brain development. Everolimus is a potent mTOR inhibitor for both reducing tumor lesions and treating intractable epilepsy in TSC. However, only 50% of patients show a clinically relevant reduction in seizure frequency and no evidence supports a positive impact for reducing cognitive and neuropsychiatric deficits in patients. If patients are not responsive to rapalogs or antiseizure medications, then surgical resection of brain lesions is the standard-of-care, however this option also has risks including acute morbidity and surgery-associated complications. Based on all of these challenges and limitations, there is a desperate and unmet clinical need to develop long lasting and highly efficacious treatment options for TSC.

Adeno-associated viruses (AAVs) have proven to be the safest and most efficacious gene delivery vectors for a variety of genetic diseases. These vectors provide multiple advantages as gene therapy vehicles which include minimal-to-no toxicity, sustained gene expression in post mitotic cells, and their ability to be administered systemically as a whole-body gene replacement treatment. Importantly, AAV-mediated gene therapy provides the value of a potentially one-time curative treatment for patients. However, a current limitation of AAV vectors is their restricted gene packaging capacity of 4.7 kilobases (kb), which must include space not only for delivering the normal copy of the gene, but also space for associated regulatory and packaging sequences. Thus, the restricted packaging capacity of AAV vectors can pose a significant challenge as a gene therapy for diseases caused by mutations in large genes. This challenge holds true for TSC type 2, as the complementary DNA sequence for TSC2 is 5.4 kb, far exceeding the size capacity of AAV vectors. Thus, alternative strategies are necessary for devising an efficacious gene replacement therapy for TSC patients with TSC2 mutations.

### SUMMARY OF THE DISCLOSURE

An aspect of the disclosure relates to an engineered micro-Tuberin comprising, from the amino to the carboxy terminus: (i) a hamartin interface domain, (ii) a GTPase-activating protein (GAP) extension domain, (iii) a first tuberin dimerization interface domain, (iv) a second tuberin dimerization interface domain, and (v) a GTPase-activating protein (GAP) domain, wherein the total length of the engineered micro-Tuberin is less than about 1300 amino acids.

In some embodiments, the micro-Tuberin does not include unstructured regions of the wild type tuberin protein as shown by SEQ ID NO: 1.

In some embodiments, the unstructured regions comprise: (a) amino acids 647-687 of SEQ ID NO: 1, (b) amino acids 924-1015 of SEQ ID NO: 1, (c) amino acids 1087-1182 of SEQ ID NO: 1, (d) amino acids 1216-1493 of SEQ ID NO: 1, and (e) amino acids 1765-1807 of SEQ ID NO: 1.

In some embodiments, the hamartin interface domain comprises: (1) at least 85% sequence identity to amino acids 94-575 of SEQ ID NO: 1, (2) at least 85% sequence identity to amino acids 94-575 and 580-646 of SEQ ID NO: 1, (3) at least 85% sequence identity to each of amino acids 94-188, 230-267, 355-419, 459-520, and 580-646 of SEQ ID NO: 1, (4) at least 85% sequence identity to amino acids 50-374, and 580-646 of SEQ ID NO: 1, (5) at least 85% sequence identity to amino acids 271-646 of SEQ ID NO: 1, (6) at least 85% sequence identity to amino acids 72-435 and 577-646 of SEQ ID NO: 1, or (7) at least 85% sequence identity to amino acids 1-646 of SEQ ID NO: 1.

In some embodiments, the GTPase-activating protein (GAP) extension domain comprises: (1) at least 85% sequence identity to amino acids 815-923 of SEQ ID NO: 1, and (2) at least 85% sequence identity to amino acids 687-923 of SEQ ID NO: 1.

In some embodiments, the first tuberin dimerization interface domain comprises at least 85% sequence identity to amino acids 1016-1086 of SEQ ID NO: 1.

In some embodiments, the second tuberin dimerization interface domain comprises at least 85% sequence identity to amino acids 1183-1215 of SEQ ID NO: 1.

In some embodiments, the GAP domain comprises at least 85% sequence identity to amino acids 1494-1764 of SEQ ID NO: 1.

In some embodiments, the micro-Tuberin further comprises a flexible linker region between each of the domains (i)-(v).

In some embodiments, the flexible linker region is selected from a peptide selected from SEQ ID NOs: 42-49.

In some embodiments, the micro-Tuberin sequence comprises an amino acid sequence selected from SEQ ID NOs: 11, 16, 21, 26, 31, 36, or 41.

In some embodiments, the micro-Tuberin further comprises a detectable label.

Another aspect of the disclosure is directed to a polynucleotide encoding the engineered micro-Tuberin of the instant disclosure, or a complement thereof, that is optionally detectably labeled.

Another aspect of the disclosure is directed to a polynucleotide comprising at least 85% sequence identity to a micro-Tuberin coding sequence selected from SEQ ID NOs: 7, 8, 12, 13, 17, 18, 22, 23, 27, 28, 32, 33, 37, or 38, optionally wherein the micro-Tuberin coding sequence comprises SEQ ID NOs: 7 or 8, optionally wherein the micro-Tuberin coding sequence comprises SEQ ID NOs: 12 or 13, optionally wherein the micro-Tuberin coding sequence comprises SEQ ID NOs: 17 or 18, optionally wherein the micro-Tuberin coding sequence comprises SEQ ID NOs: 22 or 23, optionally wherein the micro-Tuberin coding sequence comprises SEQ ID NOs: 27 or 28, optionally wherein the micro-Tuberin coding sequence comprises SEQ ID NOs: 32 or 33, or optionally wherein the micro-Tuberin coding sequence comprises SEQ ID NOs: 37 or 38.

In some embodiments, the polynucleotide further comprises a Kozak sequence (GCCACC) immediately 5′ of the micro-Tuberin coding sequence, and optionally further comprising at least one linker sequence selected from SEQ ID NOs: 50-57.

In some embodiments, the polynucleotide further comprises an Inverted terminal repeat (ITR) sequence, a promoter sequence, a 3′ UTR sequence, a polyA sequence, and a 3′ Inverted terminal repeat (ITR) sequence.

In some embodiments, the ITR sequence comprises SEQ ID NO: 2, optionally wherein the promoter sequence comprises SEQ ID NO: 3, the optionally wherein 3′ UTR sequence comprises SEQ ID NO: 4, optionally wherein the polyA sequence comprises SEQ ID NO: 5, and optionally wherein the 3′ UTR sequence comprises SEQ ID NO: 6.

In some embodiments, the polynucleotide comprises at least 85% sequence identity to a sequence selected from SEQ ID NOs: 9, 10, 14, 15, 19, 20, 24, 25, 29, 30, 34, 35, 39, or 40; optionally wherein the polynucleotide comprises SEQ ID NO: 9 or SEQ ID NO: 10, optionally wherein the polynucleotide comprises SEQ ID NO: 14 or SEQ ID NO: 15, optionally wherein the polynucleotide comprises SEQ ID NO: 19 or SEQ ID NO: 20, optionally wherein the polynucleotide comprises SEQ ID NO: 24 or SEQ ID NO: 25, optionally wherein the polynucleotide comprises SEQ ID NO: 29 or SEQ ID NO: 30, optionally wherein the polynucleotide comprises SEQ ID NO: 34 or SEQ ID NO: 35, or optionally wherein the polynucleotide comprises SEQ ID NO: 39 or SEQ ID NO: 40.

Another aspect of the disclosure is directed to a vector comprising the polynucleotide of the instant disclosure, that is optionally operationally linked to at least one regulatory element that is further optionally detectably labeled, and further optionally wherein the vector is an AAV vector.

In some embodiments, the vector is an AAV vector, and the AAV vector comprises a capsid selected from AAV1, AAV2, AAV3, AAV4, AAV5, AAV6, AAV7, AAV8, AAV9, AAV10, AAV11, AAV12, AAV13 or AAV rh74 capsid, or a variant thereof.

Another aspect of the disclosure is directed to a cell comprising the engineered micro-Tuberin of the instant disclosure, or the polynucleotide of the instant disclosure, or the vector of the instant disclosure, optionally wherein the cell is a brain cell, heart cell, kidney cell, skin cell, muscle cell or lung cell.

Another aspect of the disclosure is directed to a composition comprising the engineered micro-Tuberin of any one of claims 1-12, or the polynucleotide of any one of claims 13, 15-19, or the vector of claim 14, 20 or 21 or the cell of claim 22.

Another aspect of the disclosure is directed to a method for delivering a micro-Tuberin polynucleotide or polypeptide to a cell comprising contacting the cell with the engineered micro-Tuberin of the instant disclosure, or the polynucleotide of the instant disclosure, or the vector of the instant disclosure.

In some embodiments, the contacting is in vitro or in vivo.

In some embodiments, the cell is a mammalian cell, optionally a human cell and further optionally wherein the cell is a brain cell, heart cell, kidney cell, skin cell, muscle cell or lung cell.

Another aspect of the disclosure is directed to a method of treating a subject suffering from mTOR hyperactivation comprising administering to the subject the engineered micro-Tuberin of the instant disclosure, or the polynucleotide of the instant disclosure, or the vector of the instant disclosure.

In some embodiments, the disease is selected from the group consisting of tuberous sclerosis complex (TSC) type 2, focal cortical dysplasia type 2, lymphangioleiomyomatosis, and renal angiomyolipoma.

In some embodiments, the administration is achieved systemically, intravascularly, intracerebrally, or intrathecally; or optionally the micro-Tuberin administration is into the renal artery or vein, or into the lungs.

In some embodiments, the administration is achieved by an AAV vector, and wherein the AAV vector comprises a capsid selected from AAV1, AAV2, AAV3, AAV4, AAV5, AAV6, AAV7, AAV8, AAV9, AAV10, AAV11, AAV12, AAV13 or AAV rh74 capsid, optionally wherein the AAV vector is parenterally administered by injection, infusion or implantation.

In some embodiments, the method further comprises administering an mTOR inhibitor; optionally wherein the mTOR inhibitor is selected from rapamycin, pimecrolimus, tacrolimus, sirolimus, temsirolimus, everolimus, ridaforolimus, samimod, AZD8055, PF04691502, SF1126, XL765, or pharmaceutically acceptable salts and combinations thereof.

In some embodiments, the subject is a mammal, optionally a human.

### BRIEF DESCRIPTION OF THE DRAWINGS

FIG. 1. Tuberin, Hamartin, and TBCD17 form a complex, which negatively regulates the mTOR signaling pathway. In wild type or normal cells, the GTPase activating protein (GAP) domain of Tuberin hydrolyzes Rheb-GTP to the inactive GDP form, thereby reducing the interaction with mTOR, which controls cell proliferation by regulating downstream factors critical for ribosomal biogenesis and protein synthesis. Loss of function variants in either TSC1 or TSC2 result in hyperactivation of mTOR signaling, which causes downstream phosphorylation of S6K and S6 and ultimately increased cell growth and proliferation.

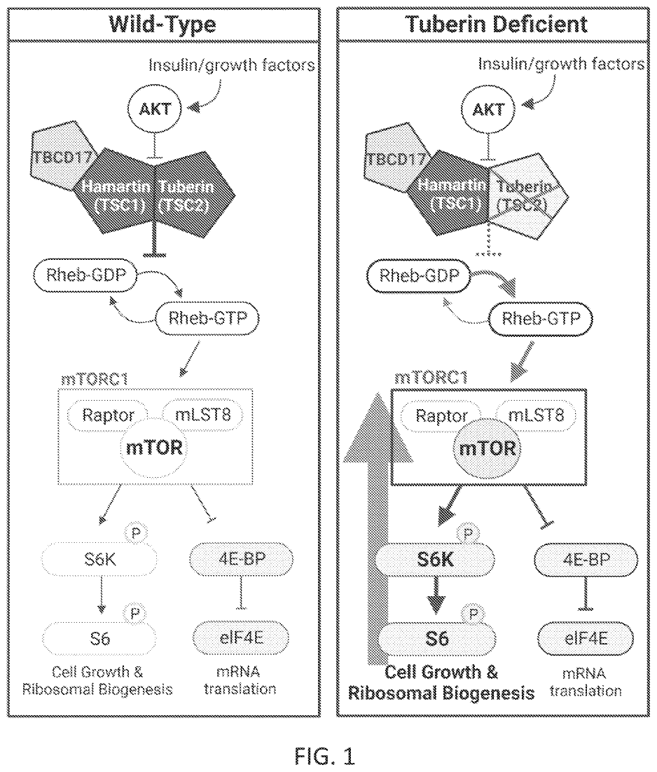

FIGS. 2A-2D. cTuberin lacks a dimerization interfacial region and overall structural homology to that of full-length (FL) Tuberin. (A) CryoEM structure of the TSC complex with two monomers of FL-Tuberin (cyan and green) and two monomers of hamartin (yellow and magenta). (B) Overlay of one monomer of FL-Tuberin (green) with the Alphafold 2 predicted structure of cTuberin (blue) zoomed to the Tuberin dimerization interface demonstrating that cTuberin lacks the interfacial region. (C) Overlay of the amino terminal region of one molecule of cTuberin (blue, far right) and the carboxy terminal region of another molecule of cTuberin (left) with the corresponding regions in FL-Tuberin (green) hiding the regions of each that were not used in the alignment to display the distance between the conserved regions (118.6 Å) indicated by the red line, demonstrating that the 16 residue flexible linker of cTuberin is insufficient to traverse this distance (typical span is 3.5 Å/residue). (D) Overlay of the amino terminal region of cTuberin (blue) with FL-Tuberin (green) displaying the entire molecule demonstrating lack of structural similarity to multiple features.

FIG. 3. Protein sequence alignment of full-length Tuberin, cTuberin, and micro-Tuberin (MT) candidates. Top schematic shows full-length Tuberin (1807 amino acids) containing various boxed domains critical for function and physiological interactions with other members in the TSC complex. Blue areas denote homologous regions to full-length Tuberin and grey areas denote absent regions in cTuberin and MT candidates. Of note, MT-2 was determined to contain an inadvertent insertion of 4 amino acids at position 281, which was subsequently characterized in downstream in vitro analyses. The MT-2 candidate shown in the alignment depicts the correct sequence. Additionally, MT-8 and MT-9 have been designed but have yet to be tested for functional activity in vitro. Glycine-serine flexible linkers of varying lengths were engineered to bridge devoid areas by measuring the intervening distance of the retained regions and inserting the appropriate number of residues to span that distance using a value of 3.5 Å/residue. MT9 represents a candidate containing all of the structured regions encoded within full-length Tuberin.

FIG. 4A-4C. Micro-Tuberin 2, 3, and 5 significantly inhibit phosphorylation of S6K at position T389. (A) Experimental strategy to transfect micro-Tuberin candidates or an EGFP control vector in conjunction with TSC1 and S6K1 into HEK-293 cells. After culturing transfected cells for 48 hours, cell lysates were processed for ELISA to detect phosho-S6K1 and total S6K1 levels among all treatment and control groups. (B) Quantification of phosphorylated S6K1 (position T389) levels, which were then normalized to total S6K1 levels. (C) Data as shown in panel B, but depicted as a percentage of activity relative to wild type (WT) or full-length Tuberin. All data are shown as the mean±SD. Statistical analyses were performed using one-way ANOVA with Tukey's multiple comparisons test. *P≤0.05; ** P≤0.01 *** P≤0.001; **** P≤0.0001; ns=not significant.

FIG. 5A-5E. CRISPR/Cas9 strategy to disrupt both TSC2 alleles in normal iPSCS. (A) TSC2 genomic locus showing exons depicted as blue bars, introns as gray lines, exons encoding the Hamartin interacting region as a red line, and exons encoding the GTPase activating protein (GAP) domain as a dark gray line. Two small guide (sg)RNAs were designed either in intron 1 (sgRNA1) or in intron 4 (sgRNA2) to delete a 4.9 kilobase (kb) region that spans exons 2-4, a knockout (KO) strategy that is synonymous to a Tsc2 preclinical conditional knockout mouse model. Excision of exons 2-4 causes a frameshift and a subsequent premature stop codon at c.542-544 causing nonsense mediated decay. (B-C) Phase contrast images of isogenic control iPSCs and TSC2 KO iPSCs. (D-E) Western blot analysis showing loss of Tuberin in TSC2 KO iPSCs and increased mTORC1 signaling as demonstrated by increased pS6K1 levels.

FIG. 6A-6D. AAV9 Micro-Tuberin candidates differentially inhibit mTORC1 signaling, which causes a reduction of pS6 levels in TSC2 knockout astrocytes. (A) Schematic of the AAV9 vector backbone containing micro-Tuberin candidates, a 225 base pair 3′ TSC2 untranslated region (UTR), and 208 base pair bovine growth hormone polyadenylation (BGH-pA) signal. Expression of micro-Tuberin transgenes is driven by the 850 base pair chicken beta-actin (CBA) promoter. EGFP control and micro-Tuberin candidates were packaged into AAV9 viral vectors for subsequent infection of TSC2 knockout astrocytes, which were allowed to culture for 7 days and then assessed for pS6 levels (at positions Ser235/236) by immunofluorescence (IF) analysis. (B) Representative images of pS6 IF staining (red) in all treatment and control groups, DAPI was used to stain nuclei (blue). (C-D) Quantification of relative pS6 fluorescence intensity and pS6 area occupancy in control and treatment groups. All data are shown as the mean±SD. Statistical analyses were performed using one-way ANOVA with Tukey's multiple comparisons test. Statistical significance was achieved in all treatment groups in comparison to the mock EGFP control *** P≤0.001.

FIG. 7. MT-9 shows near-exact structural homology to that of full-length Tuberin. Protein structure model showing the overlay of the AlphaFold 2 model of MT-9 (red) with the CryoEM structure (7DL2) of full-length Tuberin (green) demonstrating near-exact similarity in structural features of full-length Tuberin including fully intact GAP domain, Tuberin dimerization interface, GAP extensions, and Hamartin interfaces.

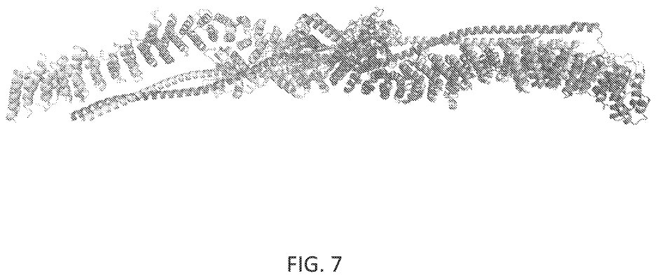

FIG. 8. AlphaFold 2 models and SwissModel homology models of each of the micro-Tuberin candidates. The AlphaFold 2 models often do not overlay as well because the folding is based on sequence only and the flexible linkers will have no structure so the orientation of the next folded region may not be in the correct orientation. The SwissModel homology models were created using the 7DL2 structure as the template and demonstrate that the flexible linkers are long enough to allow the folded regions to directly overlap with the corresponding regions in the cryoEM structure.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

The detailed description is divided into various sections only for the reader's convenience and disclosure found in any section may be combined with that in another section. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

As will be understood by one skilled in the art, for any and all purposes, all ranges disclosed herein also encompass any and all possible subranges and combinations of subranges thereof. Furthermore, as will be understood by one skilled in the art, a range includes each individual member.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

### Definitions

As it would be understood, the section or subsection headings as used herein is for organizational purposes only and are not to be construed as limiting and/or separating the subject matter described.

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The term “about” when used before a numerical designation, e.g., temperature, time, amount, concentration, and such other, including a range, indicates approximations which may vary by (+) or (−) 10%, 5% or 1%.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has, or is diagnosed of having, or is suspected of having, or is at risk of having a disease, such as a cancer or a hereditary disease such as sickle cell anemia or cystic fibrosis.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the composition or cell to provide the therapeutic benefit in vitro or in vivo by at least 10%, 25%, 40%, 60%, 80%, 90% or 95% as compared to control. Determination of these parameters is well within the skill of the art. These considerations, as well as effective formulations and administration procedures are well known in the art and are described in standard textbooks.

Administration or delivery in vivo can be effected in one dose, continuously or intermittently throughout the course of treatment. Methods of determining the most effective means and dosage of administration are well known to those of skill in the art and will vary with the composition used for therapy, the purpose of the therapy, the target cell, solid tumor, or cancer being treated, and the subject being treated. Single or multiple administrations can be carried out with the dose level and pattern being selected by the treating physician.

The pharmaceutical compositions can be administered by inhalation, orally, intranasally, parenterally, injection, orally and may take the form of tablets, lozenges, granules, capsules, pills, ampoules, suppositories, or aerosol form. They may also take the form of suspensions, solutions, and emulsions of the active ingredient in aqueous or nonaqueous diluents, syrups, granulates or powders. In addition to an agent of the present disclosure, the compositions can also contain other pharmaceutically active compounds or a plurality of systems or cells of the disclosure.

More particularly, an agent of the present disclosure also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

While it is possible for the agent to be administered alone, it is preferable to present it as a pharmaceutical formulation comprising, or consisting essentially of, or consisting of at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present disclosure suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the agent, such carriers as are known in the art to be appropriate.

Formulations suitable for nasal administration or aerosol (directly into the lung), wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above, the formulations of this disclosure may include other agents conventional in the art having regard to the type of formulation in question, for example, those suitable for oral administration may include such further agents as sweeteners, thickeners and flavoring agents. It also is intended that the agents, compositions and methods of this disclosure be combined with other suitable compositions and therapies.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides, ribonucleotides, hybrid polynucleotides or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones. A polynucleotide may comprise one or more modified nucleotides, such as methylated nucleotides and nucleotide analogs or those as described herein. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “vector” refers to a construct which is capable of delivering, and, in some embodiments expressing, a polynucleotide into a cell. Non-limiting examples of delivery vectors include viral vectors, nucleic acid expression vectors (such as a plasmid), naked DNA, and certain eukaryotic cells (e.g., producer cells). In some embodiments, nucleic acids described by the disclosure are delivered via a viral vector. Examples of viral vectors include retroviral vectors (e.g., Maloney murine leukemia virus, MML-V), adenoviral vectors (e.g., AD 100), lentiviral vectors (e.g., HIV and FIV-based vectors), and herpesvirus vectors (e.g., HSV, HSV-1, HSV-2), as described by Chira et al. (Oncotarget, 2015, 6(31); 30673-30703). In some embodiments, nucleic acids described by the disclosure are delivered by an adeno-associated virus (AAV) vector (e.g., a recombinant AAV (rAAV) vector).

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques in Biochemistry and Molecular Biology-Hybridization with Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridising to the reference sequence under highly stringent conditions. Generally, in order to maximize the hybridization rate, relatively low-stringency hybridization conditions are selected: about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately-stringent washing conditions are selected to be about 15 to 30° C. lower than the Tₘ. Highly permissive (very low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. Exemplary highly stringent conditions comprise incubation in 50% formamide, 5×SSC, and 1% SDS at 42° C., or incubation in 5×SSC and 1% SDS at 65° C., with wash in 0.2×SSC and 0.1% SDS at 65° C.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogsteen base pairing, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “domain” or “protein domain” refers to a part of a protein sequence that may exist and function independently of the rest of the protein chain.

As described in aspects of this disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., gene-editing system transcripts, proteins, enzymes, mutant forms thereof, fusion proteins thereof, etc.). With regards to regulatory sequences, mention is made of U.S. patent application Ser. No. 10/491,026, the contents of which are incorporated by reference herein in their entirety. With regards to promoters, mention is made of PCT publication WO 2011/028929 and U.S. application Ser. No. 12/511,940, the contents of which are incorporated by reference herein in their entirety.

Vectors can be designed for expression of gene-editing system transcripts (e.g. nucleic acid transcripts, proteins, or enzymes) in prokaryotic or eukaryotic cells. For example, gene-editing system transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerevisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments of the disclosure may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety.

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, pl9, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8: 659-669 (1997); Kessler et al., Proc Nat. Acad Sc. USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the disclosure comprise, or consist essentially of, or yet further consist of a nucleic acid molecule encoding micro-Tuberin and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art. To promote skeletal muscle specific expression, AAV1, AAV5, AAV6, AAV8 or AAV9 may be used.

In some embodiments, a regulatory element is operably linked to one or more elements of a gene-editing system so as to drive expression of the one or more elements of the gene-editing system.

The gene-editing systems described herein can further comprise one or more labels or detection tags (e.g., FLAG™ tag, epitope or protein tags, such as myc tag, 6 His, and fluorescent fusion protein). In an aspect, the label (e.g., FLAG™ tag) is fused to the NLS. In an aspect, the disclosed methods and compositions further comprise a fusion protein, or a polynucleotide encoding the same. In various aspects, the fusion protein comprises at least one epitope-providing amino acid sequence (e.g., “epitope-tag”), wherein the epitope-tag is selected from i) an epitope-tag added to the N- and/or C-terminus of a protein, or ii) an epitope-tag inserted into a region of a protein, and an epitope-tag replacing a number of amino acids in a protein.

As used herein, “epitope tags” refer to short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In an aspect, the disclosed methods and compositions comprise an epitope-tag wherein the epitope-tag has a length of between 6 to 15 amino acids. In an alternative aspect, the epitope-tag has a length of 9 to 11 amino acids. The disclose methods and compositions can also comprise a fusion protein comprising two or more epitope-tags, either spaced apart or directly in tandem. Further, the disclosed methods and composition can comprise 2, 3, 4, 5 or even more epitope-tags, as long as the fusion protein maintains its biological activity/activities (e.g., “functional”).

In an aspect, the epitope-tag is a VSV-G tag, CD tag, calmodulin-binding peptide tag, S-tag, Avitag, SF-TAP-tag, strep-tag, myc-tag, FLAG-tag, T7-tag, HA (hemagglutinin)-tag, His-tag, S-tag, GST-tag, or GFP-tag. The sequences of these tags are described in the literature and well known to the person of skill in art.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

As used herein, the term “detectably labeled” means that the agent (biologic or small molecule) is attached to another molecule, compound or polymer that facilitates detection of the presence of the agent in vitro or in vivo. A “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

### Modes for Carrying Out the Disclosure

The instant disclosure is generally directed to a novel gene replacement strategy to treat tuberous sclerosis complex (TSC) type 2. In some aspects, the gene replacement strategy uses a viral delivery vector, such as an adeno-associated viral (AAV) vector. Since the coding region of TSC2 (~5.4 kb) far exceeds the packaging capacity of current AAV vectors, Applicant has leveraged principles based on rational protein design and artificial-intelligence-based protein structure prediction in order to develop micro-Tuberin (MT) candidates with the same key structural motifs and functional components as encoded in full-length Tuberin. Based on these principles, novel micro-Tuberin candidates were generated, which were validated by homology modeling using Swiss model and the artificial intelligence (AI)-guided folding prediction tool, Alphafold 2. The in vitro efficacy studies demonstrated that the novel micro-Tuberin polypeptides can negatively regulate mTOR signaling as well as full length (wild type) tuberin. Also disclosed herein are methods for delivering a micro-Tuberin polynucleotide or a polypeptide to a cell and methods for treating tuberous sclerosis complex (TSC) type 2 by administering a micro-Tuberin polynucleotide or a polypeptide to a subject in need thereof.

### Engineered Micro-Tuberins

An aspect of the disclosure is directed to an engineered micro-Tuberin comprising, or consisting essentially of, or consisting of, from the amino to the carboxy terminus: (i) a hamartin interface domain, (ii) a GTPase-activating protein (GAP) extension domain, (iii) a first tuberin dimerization interface domain, (iv) a second tuberin dimerization interface domain, and (v) a GTPase-activating protein (GAP) domain, wherein the total length of the engineered micro-Tuberin is less than 1300 amino acids. As used herein, the term “engineered micro-Tuberin” intends a non-naturally occurring protein or polypeptide.

In some embodiment, the total length of the engineered micro-Tuberin is between 800-1300 amino acids. In some embodiment, the total length of the engineered micro-Tuberin is between about 750-1300 amino acids (e.g., about 750, 800, 850, 900, 950, 1000, 1050, 1100, 1150, 1200, 1250, or 1300) amino acids. In some embodiment, the total length of the engineered micro-Tuberin is between about 900-1250 (e.g., 900, 950, 1000, 1050, 1100, 1150, 1200, or 1250) amino acids.

In some embodiment, the engineered micro-Tuberin does not include unstructured regions of the wild type tuberin protein as shown by SEQ ID NO: 1. In some embodiments, the structured and unstructured regions of the wild type tuberin protein is as shown by the cryoEM structure PDBid: 7DL2, where the unstructured regions are shown as gaps in the cryoEM structure.

In some embodiments, the unstructured regions comprise: (a) amino acids 637-697 of SEQ ID NO: 1, (b) amino acids 914-1025 of SEQ ID NO: 1, (c) amino acids 1077-1192 of SEQ ID NO: 1, (d) amino acids 1206-1503 of SEQ ID NO: 1, and (e) amino acids 1755-1817 of SEQ ID NO: 1. In some embodiments, the unstructured regions comprise: (a) amino acids 640-695 of SEQ ID NO: 1, (b) amino acids 918-1020 of SEQ ID NO: 1, (c) amino acids 1080-1190 of SEQ ID NO: 1, (d) amino acids 1210-1500 of SEQ ID NO: 1, and (e) amino acids 1755-1815 of SEQ ID NO: 1. In some embodiments, the unstructured regions comprise: (a) amino acids 645-690 of SEQ ID NO: 1, (b) amino acids 920-1018 of SEQ ID NO: 1, (c) amino acids 1085-1185 of SEQ ID NO: 1, (d) amino acids 1215-1495 of SEQ ID NO: 1, and (e) amino acids 1760-1810 of SEQ ID NO: 1. In some embodiments, the unstructured regions comprise: (a) amino acids 647-687 of SEQ ID NO: 1, (b) amino acids 924-1015 of SEQ ID NO: 1, (c) amino acids 1087-1182 of SEQ ID NO: 1, (d) amino acids 1216-1493 of SEQ ID NO: 1, and (e) amino acids 1765-1807 of SEQ ID NO: 1.

In some embodiments, the hamartin interface domain comprises, or consists essentially of, or consists of at least 85%, at least 90%, at least 95%, at least 99% or more sequence identity to amino acids 84-585 of SEQ ID NO: 1. In some embodiments, the hamartin interface domain comprises, or consists essentially of, or consists of at least 85%, at least 90%, at least 95%, at least 99% or more sequence identity to amino acids 90-580 of SEQ ID NO: 1. In some embodiments, the hamartin interface domain comprises, or consists essentially of, or consists of at least 85%, at least 90%, at least 95%, at least 99% or more sequence identity to amino acids 94-575 of SEQ ID NO: 1.

In some embodiments, the hamartin interface domain comprises, or consists essentially of, or consists of at least 85%, at least 90%, at least 95%, at least 99% or more sequence identity to amino acids 84-585 and amino acids 570-656 of SEQ ID NO: 1. In some embodiments, the hamartin interface domain comprises, or consists essentially of, or consists of at least 85%, at least 90%, at least 95%, at least 99% or more sequence identity to amino acids 90-580 and amino acids 575-650 of SEQ ID NO: 1. In some embodiments, the hamartin interface domain comprises, or consists essentially of, or consists of at least 85%, at least 90%, at least 95%, at least 99% or more sequence identity to amino acids 94-575 and amino acids 580-646 of SEQ ID NO: 1.

In some embodiments, the hamartin interface domain comprises, or consists essentially of, or consists of at least 85%, at least 90%, at least 95%, at least 99% or more sequence identity to amino acids 84-198, 220-277, 345-429, 449-530, and 570-656 of SEQ ID NO: 1. In some embodiments, the hamartin interface domain comprises, or consists essentially of, or consists of at least 85%, at least 90%, at least 95%, at least 99% or more sequence identity to amino acids 90-193, 225-273, 350-425, 455-525, and 575-650 of SEQ ID NO: 1. In some embodiments, the hamartin interface domain comprises, or consists essentially of, or consists of at least 85%, at least 90%, at least 95%, at least 99% or more sequence identity to amino acids 94-188, 230-267, 355-419, 459-520, and 580-646 of SEQ ID NO: 1.

In some embodiments, the hamartin interface domain comprises, or consists essentially of, or consists of at least 85%, at least 90%, at least 95%, at least 99% or more sequence identity to amino acids 40-384, and 570-656 of SEQ ID NO: 1. In some embodiments, the hamartin interface domain comprises, or consists essentially of, or consists of at least 85%, at least 90%, at least 95%, at least 99% or more sequence identity to amino acids 45-380, and 575-650 of SEQ ID NO: 1. In some embodiments, the hamartin interface domain comprises, or consists essentially of, or consists of at least 85%, at least 90%, at least 95%, at least 99% or more sequence identity to amino acids 50-374, and 580-646 of SEQ ID NO: 1.

In some embodiments, the hamartin interface domain comprises, or consists essentially of, or consists of at least 85%, at least 90%, at least 95%, at least 99% or more sequence identity to amino acids 261-656 of SEQ ID NO: 1. In some embodiments, the hamartin interface domain comprises, or consists essentially of, or consists of at least 85%, at least 90%, at least 95%, at least 99% or more sequence identity to amino acids 265-650 of SEQ ID NO: 1. In some embodiments, the hamartin interface domain comprises, or consists essentially of, or consists of at least 85%, at least 90%, at least 95%, at least 99% or more sequence identity to amino acids 271-646 of SEQ ID NO: 1.

In some embodiments, the hamartin interface domain comprises, or consists essentially of, or consists of at least 85%, at least 90%, at least 95%, at least 99% or more sequence identity to amino acids 62-445 and 567-656 of SEQ ID NO: 1. In some embodiments, the hamartin interface domain comprises, or consists essentially of, or consists of at least 85%, at least 90%, at least 95%, at least 99% or more sequence identity to amino acids 65-440 and 573-650 of SEQ ID NO: 1. In some embodiments, the hamartin interface domain comprises, or consists essentially of, or consists of at least 85%, at least 90%, at least 95%, at least 99% or more sequence identity to amino acids 72-435 and 577-646 of SEQ ID NO: 1.

In some embodiments, the hamartin interface domain comprises, or consists essentially of, or consists of at least 85%, at least 90%, at least 95%, at least 99% or more sequence identity to amino acids 1-656 of SEQ ID NO: 1. In some embodiments, the hamartin interface domain comprises, or consists essentially of, or consists of at least 85%, at least 90%, at least 95%, at least 99% or more sequence identity to amino acids 1-650 of SEQ ID NO: 1. In some embodiments, the hamartin interface domain comprises, or consists essentially of, or consists of at least 85%, at least 90%, at least 95%, at least 99% or more sequence identity to amino acids 1-646 of SEQ ID NO: 1.

In some embodiments, the GTPase-activating protein (GAP) extension domain comprises, or consists essentially of, or consists of at least 85%, at least 90%, at least 95%, at least 99% or more sequence identity to amino acids 805-933 of SEQ ID NO: 1. In some embodiments, the GTPase-activating protein (GAP) extension domain comprises, or consists essentially of, or consists of at least 85%, at least 90%, at least 95%, at least 99% or more sequence identity to amino acids 810-927 of SEQ ID NO: 1. In some embodiments, the GTPase-activating protein (GAP) extension domain comprises, or consists essentially of, or consists of at least 85%, at least 90%, at least 95%, at least 99% or more sequence identity to amino acids 815-923 of SEQ ID NO: 1.

In some embodiments, the GTPase-activating protein (GAP) extension domain comprises, or consists essentially of, or consists of at least 85%, at least 90%, at least 95%, at least 99% or more sequence identity to amino acids 677-933 of SEQ ID NO: 1. In some embodiments, the GTPase-activating protein (GAP) extension domain comprises, or consists essentially of, or consists of at least 85%, at least 90%, at least 95%, at least 99% or more sequence identity to amino acids 683-928 of SEQ ID NO: 1. In some embodiments, the GTPase-activating protein (GAP) extension domain comprises, or consists essentially of, or consists of at least 85%, at least 90%, at least 95%, at least 99% or more sequence identity to amino acids 687-923 of SEQ ID NO: 1.

In some embodiments, the first tuberin dimerization interface domain comprises, or consists essentially of, or consists of at least 85%, at least 90%, at least 95%, at least 99% or more sequence identity to amino acids 1006-1096 of SEQ ID NO: 1. In some embodiments, the first tuberin dimerization interface domain comprises, or consists essentially of, or consists of at least 85%, at least 90%, at least 95%, at least 99% or more sequence identity to amino acids 1010-1090 of SEQ ID NO: 1. In some embodiments, the first tuberin dimerization interface domain comprises, or consists essentially of, or consists of at least 85%, at least 90%, at least 95%, at least 99% or more sequence identity to amino acids 1016-1086 of SEQ ID NO: 1.

In some embodiments, the second tuberin dimerization interface domain comprises, or consists essentially of, or consists of at least 85%, at least 90%, at least 95%, at least 99% or more sequence identity to amino acids 1173-1225 of SEQ ID NO: 1. In some embodiments, the second tuberin dimerization interface domain comprises, or consists essentially of, or consists of at least 85%, at least 90%, at least 95%, at least 99% or more sequence identity to amino acids 1178-1220 of SEQ ID NO: 1. In some embodiments, the second tuberin dimerization interface domain comprises, or consists essentially of, or consists of at least 85%, at least 90%, at least 95%, at least 99% or more sequence identity to amino acids 1183-1215 of SEQ ID NO: 1.

In some embodiments, the GAP domain comprises, or consists essentially of, or consists of at least 85%, at least 90%, at least 95%, at least 99% or more sequence identity to amino acids 1494-1764 of SEQ ID NO: 1. In some embodiments, the GAP domain comprises, or consists essentially of, or consists of at least 85%, at least 90%, at least 95%, at least 99% or more sequence identity to amino acids 1494-1764 of SEQ ID NO: 1. In some embodiments, the GAP domain comprises, or consists essentially of, or consists of at least 85%, at least 90%, at least 95%, at least 99% or more sequence identity to amino acids 1494-1764 of SEQ ID NO: 1.

In some embodiments, the engineered micro-Tuberin comprises a flexible linker region between domains, and between the individual components of the domains. In some embodiments, the flexible linker length is adjusted to position domains of the engineered micro-Tuberin correctly with pairing hamartin. In some embodiments, the flexible linker is selected from a peptide selected from SEQ ID NOs: 42-49.

In some embodiments, the engineered tuberin comprises, in the following order, (i) a hamartin interface domain, (ii) a GTPase-activating protein (GAP) extension domain, (iii) a first tuberin dimerization interface domain, (iv) a second tuberin dimerization interface domain, and (v) a GTPase-activating protein (GAP) domain, and there is a flexible linker between (i) and (ii) that is at least 3 amino acids long and at most 20 amino acids long (e.g., 3, 4, 5, 6, 7, 8, 9, 10, 11, 12, 13, 14, 15, 16, 17, 18, 19, or 20 amino acids long), there is a flexible linker between (ii) and (iii) that is at least 3 amino acids long and at most 20 amino acids long (e.g., 3, 4, 5, 6, 7, 8, 9, 10, 11, 12, 13, 14, 15, 16, 17, 18, 19, or 20 amino acids long), there is a flexible linker between (iii) and (iv) that is at least 5 amino acids long and at most 20 amino acids long (e.g., 5, 6, 7, 8, 9, 10, 11, 12, 13, 14, 15, 16, 17, 18, 19, or 20 amino acids long), and there is a flexible linker between (iv) and (v) that is at least 3 amino acids long and at most 20 amino acids long (e.g., 3, 4, 5, 6, 7, 8, 9, 10, 11, 12, 13, 14, 15, 16, 17, 18, 19, or 20 amino acids long).

In some embodiments, the engineered micro-Tuberin comprises amino acids 94-575, 815-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 94-575, (b) 815-923, (c) 1016-1086, (d) 1183-1215 and (e) 1494-1764 of SEQ ID NO: 1. In some embodiments, there is a flexible linker between each of (a) through (e) (i.e., there is a flexible linker between (a) and (b), a flexible linker between (b) and (c), a flexible linker between (c) and (d), and a flexible linker between (d) and (e)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 11. In a specific embodiment, the engineered micro-Tuberin comprises SEQ ID NO: 11.

In some embodiments, the engineered micro-Tuberin comprises amino acids 94-575, 580-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 94-575, (b) 580-646, (c) 687-923, (d) 1016-1086, (e) 1183-1215 and (f) 1494-1764 of SEQ ID NO: 1. In some embodiments, there is a flexible linker between each of (a) through (f) that is at least 3 amino acids long and at most 20 amino acids long (i.e., there is a flexible linker between (a) and (b), a flexible linker between (b) and (c), a flexible linker between (c) and (d), a flexible linker between (d) and (e), and a flexible linker between (e) and (f), each of which is 3, 4, 5, 6, 7, 8, 9, 10, 11, 12, 13, 14, 15, 16, 17, 18, 19, or 20 amino acids long). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 16. In a specific embodiment, the engineered micro-Tuberin comprises SEQ ID NO: 16.

In some embodiments, the engineered micro-Tuberin comprises amino acids 94-188, 230-267, 355-419, 459-520, 580-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 94-188, (b) 230-267, (c) 355-419, (d) 459-520, (e) 580-646, (f) 687-923, (g) 1016-1086, (h) 1183-1215 and (i) 1494-1764 of SEQ ID NO: 1. In some embodiments, there is a flexible linker between each of (a) through (i) (i.e., there is a flexible linker between (a) and (b), a flexible linker between (b) and (c), a flexible linker between (c) and (d), a flexible linker between (d) and (e), a flexible linker between (e) and (f), a flexible linker between (g) and (h), and a flexible linker between (h) and (i)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 21. In a specific embodiment, the engineered micro-Tuberin comprises SEQ ID NO: 21.

In some embodiments, the engineered micro-Tuberin comprises amino acids 50-374, 580-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 50-374, (b) 580-646, (c) 687-923, (d) 1016-1086, (e) 1183-1215 and (f) 1494-1764 of SEQ ID NO: 1. In some embodiments, there is a flexible linker between each of (a) through (f) (i.e., there is a flexible linker between (a) and (b), a flexible linker between (b) and (c), a flexible linker between (c) and (d), a flexible linker between (d) and (e), and a flexible linker between (e) and (f)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 26. In a specific embodiment, the engineered micro-Tuberin comprises SEQ ID NO: 26.

In some embodiments, the engineered micro-Tuberin comprises amino acids 271-435, 577-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 271-435, (b) 577-646, (c) 687-923, (d) 1016-1086, (e) 1183-1215 and (f) 1494-1764 of SEQ ID NO: 1. In some embodiments, there is a flexible linker between each of (a) through (f) (i.e., there is a flexible linker between (a) and (b), a flexible linker between (b) and (c), a flexible linker between (c) and (d), a flexible linker between (d) and (e), and a flexible linker between (e) and (f)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 31. In a specific embodiment, the engineered micro-Tuberin comprises SEQ ID NO: 31.

In some embodiments, the engineered micro-Tuberin comprises amino acids 72-435, 577-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 72-435, (b) 577-646, (c) 687-923, (d) 1016-1086, (e) 1183-1215 and (f) 1494-1764 of SEQ ID NO: 1. In some embodiments, there is a flexible linker between each of (a) through (f) (i.e., there is a flexible linker between (a) and (b), a flexible linker between (b) and (c), a flexible linker between (c) and (d), a flexible linker between (d) and (e), and a flexible linker between (e) and (f)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 36. In a specific embodiment, the engineered micro-Tuberin comprises SEQ ID NO: 36.

In some embodiments, the engineered micro-Tuberin comprises amino acids 1-646, 687-923, 1016-1086, 1183-1215 and 1494-1764 of SEQ ID NO: 1. In some embodiments, the engineered micro-Tuberin comprises, or consists essentially of, or consists of at least 85%, at least 90%, at least 95%, at least 99% or more sequence identity to a polypeptide comprising, in the following order, a plurality of domains having amino acids (a) 1-646, (b) 687-923, (c) 1016-1086, (d) 1183-1215 and (e) 1494-1764 of SEQ ID NO: 1. In some embodiments, there is a flexible linker between each of (a) through (e) (i.e., there is a flexible linker between (a) and (b), a flexible linker between (b) and (c), a flexible linker between (c) and (d), and a flexible linker between (d) and (e)). In some embodiments, the flexible linker is selected from a peptide selected from SEQ ID NOs: 42-49. In some embodiments, the length of the flexible linker between different domains of the engineered micro-Tuberin is determined by the relative distance between the domains in wild type micro-Tuberin protein. In some embodiments, the engineered micro-Tuberin comprises, or consists essentially of, or consists of at least 85%, at least 90%, at least 95%, at least 99% or more sequence identity to SEQ ID NO: 41. In a specific embodiment, the engineered micro-Tuberin comprises SEQ ID NO: 41.

In some embodiment, the engineered micro-Tuberin of the instant disclosure comprises a detectable label.

### Polynucleotides Encoding Micro-Tuberins

Another aspect of the disclosure is directed to a polynucleotide encoding the engineered micro-Tuberin disclosed herein. In some embodiments, the polynucleotide is detectably labeled. In some embodiment, the polynucleotide is codon optimized. In some embodiments, the polynucleotide is codon optimized for expression in a mammalian cell.

In some embodiments, the polynucleotide comprises, or consists essentially of, or consists of at least 85%, at least 90%, at least 95%, at least 99% or more sequence identity to a micro-Tuberin coding sequence selected from SEQ ID NOs: 7, 8, 12, 13, 17, 18, 22, 23, 37, 38, 32, 33, 37, or 38.

In some embodiments, the micro-Tuberin coding sequence comprises, or consists essentially of, or consists of SEQ ID NOs: 7 or 8. In some embodiments, the micro-Tuberin coding sequence comprises, or consists essentially of, or consists of SEQ ID NOs: 12 or 13. In some embodiments, the micro-Tuberin coding sequence comprises, or consists essentially of, or consists of SEQ ID NOs: 17 or 18. In some embodiments, the micro-Tuberin coding sequence comprises, or consists essentially of, or consists of SEQ ID NOs: 22 or 23. In some embodiments, the micro-Tuberin coding sequence comprises, or consists essentially of, or consists of SEQ ID NOs: 27 or 28. In some embodiments, the micro-Tuberin coding sequence comprises, or consists essentially of, or consists of SEQ ID NOs: 32 or 33. In some embodiments, the micro-Tuberin coding sequence comprises, or consists essentially of, or consists of SEQ ID NOs: 37 or 38.

In some embodiments, the polypeptide further comprises a Kozak sequence comprising, consisting essentially of, or consisting of GCCACC immediately 5′ of the micro-Tuberin coding sequence.

In some embodiments, the polypeptide further comprises at least one linker sequence selected from SEQ ID No: 50-57.

In some embodiments, the polypeptide further comprises an Inverted terminal repeat (ITR) sequence. In some embodiments, the ITR sequence comprises, or consists essentially of, or consists of at least 85%, at least 90%, at least 95%, at least 99% or more sequence identity to SEQ ID NO: 2. In some embodiments, the ITR sequence comprises, or consists essentially of, or consists of SEQ ID NO: 2.

In some embodiments, the polypeptide further comprises a promoter sequence. In some embodiments, the promoter sequence comprises, or consists essentially of, or consists of at least 85%, at least 90%, at least 95%, at least 99% or more sequence identity to SEQ ID NO: 3. In some embodiments, the promoter sequence comprises, or consists essentially of, or consists of SEQ ID NO: 3.

In some embodiments, the polypeptide further comprises a 3′ UTR sequence. In some embodiments, the 3′ UTR sequence comprises, or consists essentially of, or consists of at least 85%, at least 90%, at least 95%, at least 99% or more sequence identity to SEQ ID NO: 4. In some embodiments, the 3′ UTR sequence comprises, or consists essentially of, or consists of SEQ ID NO: 4.

In some embodiments, the polypeptide further comprises a polyA sequence. In some embodiments, the polyA sequence comprises, or consists essentially of, or consists of at least 85%, at least 90%, at least 95%, at least 99% or more sequence identity to SEQ ID NO: 5. In some embodiments, the polyA sequence comprises, or consists essentially of, or consists of SEQ ID NO: 5.

In some embodiments, the polypeptide further comprises a 3′ Inverted terminal repeat (ITR) sequence. In some embodiments, the 3′ ITR sequence comprises, or consists essentially of, or consists of at least 85%, at least 90%, at least 95%, at least 99% or more sequence identity to SEQ ID NO: 6. In some embodiments, the 3′ ITR sequence comprises, or consists essentially of, or consists of SEQ ID NO: 6.

In some embodiments, the polypeptide comprises, or consists essentially of, or consists of at least 85%, at least 90%, at least 95%, at least 99% or more sequence identity to SEQ ID NO: 9 or 10. In some embodiments, the polypeptide comprises, or consists essentially of, or consists of at least 85%, at least 90%, at least 95%, at least 99% or more sequence identity to SEQ ID NO: 14 or 15. In some embodiments, the polypeptide comprises, or consists essentially of, or consists of at least 85%, at least 90%, at least 95%, at least 99% or more sequence identity to SEQ ID NO: 19 or 20. In some embodiments, the polypeptide comprises, or consists essentially of, or consists of at least 85%, at least 90%, at least 95%, at least 99% or more sequence identity to SEQ ID NO: 24 or 25. In some embodiments, the polypeptide comprises, or consists essentially of, or consists of at least 85%, at least 90%, at least 95%, at least 99% or more sequence identity to SEQ ID NO: 29 or 30. In some embodiments, the polypeptide comprises, or consists essentially of, or consists of at least 85%, at least 90%, at least 95%, at least 99% or more sequence identity to SEQ ID NO: 34 or 35. In some embodiments, the polypeptide comprises, or consists essentially of, or consists of at least 85%, at least 90%, at least 95%, at least 99% or more sequence identity to SEQ ID NO: 39 or 40.

### Vectors

Another aspect of the disclosure is directed to a vector comprising any one of the polynucleotides disclosed herein operationally linked to at least one regulatory element. In some embodiments, the polynucleotide or the vector is detectably labeled. In some embodiments, the vector is an AAV vector. In some embodiments, the AAV vector comprises a capsid selected from AAV1, AAV2, AAV3, AAV4, AAV5, AAV6, AAV7, AAV8, AAV9, AAV10, AAV11, or AAV12 capsid, or a variant thereof.

In some embodiments, DNA plasmids of the disclosure comprise AAV genomes. The DNA plasmids are transferred to cells permissible for infection with a helper virus of AAV (e.g., adenovirus, El-deleted adenovirus or herpesvirus) for assembly of the AAV genome into infectious viral particles. Techniques to produce AAV particles, in which an AAV genome to be packaged, rep and cap genes, and helper virus functions are provided to a cell are standard in the art. Production of AAV requires that the following components are present within a single cell (denoted herein as a packaging cell): an AAV genome, AAV rep and cap genes separate from (i.e., not in) the AAV genome, and helper virus functions. The AAV rep and cap genes may be from any AAV serotype for which recombinant virus can be derived and may be from a different AAV serotype than the AAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. In some embodiments, an AAV vector comprises the inverted terminal repeat (ITR) sequences of AAV2. Production of pseudotyped AAV is disclosed in, for example, WO 01/83692 which is incorporated by reference herein in its entirety. In certain aspects, an AAV vector comprises the inverted ITR sequences of AAV2 and is encapsidated in a capsid of AAV rh.

A method of generating a packaging cell line is to create a cell line that stably expresses all the necessary components for AAV particle production. For example, a plasmid (or multiple plasmids) comprising an AAV genome lacking AAV rep and cap genes, AAV rep and cap genes separate from the AAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of AAV. Other examples of suitable methods employ adenovirus or baculovirus rather than plasmids to introduce AAV genomes and/or rep and cap genes into packaging cells.

General principles of AAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62: 1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13: 1244-1250; Paul et al. (1993) Human Gene Therapy 4:609-615; Clark et al. (1996) Gene Therapy 3: 1124-1132; U.S. Pat. Nos. 5,786,211; 5,871,982; and 6,258,595. The foregoing documents are hereby incorporated by reference in their entirety herein, with particular emphasis on those sections of the documents relating to AAV production.

The disclosure thus provides packaging cells that produce infectious AAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated AAV particles) of the disclosure comprise an AAV genome.

The AAV may be purified by methods standard in the art such as by column chromatography or cesium chloride gradients. Methods for purifying AAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising, or consisting essentially of AAV of the present disclosure. Compositions described herein comprise, or consist essentially of AAV in a pharmaceutically acceptable carrier. In one particular embodiment, the composition of this disclosure comprises, or consists essentially of, or consists of Lactated Ringer's Solution (LRS). The compositions may also comprise other ingredients such as diluents and adjuvants. Acceptable carriers, diluents and adjuvants are nontoxic to recipients and are inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Capsid proteins of an AAV may be modified so that the AAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The AAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of AAV as a free acid (DNA contains acidic phosphate groups) or a pharmacologically acceptable salt can be prepared in water suitably mixed with a surfactant such as hydroxpropylcellulose. A dispersion of AAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating AAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Transduction with AAV may also be carried out in vitro. In one embodiment, desired target muscle cells are removed from the subject, transduced with AAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining AAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with AAV of the disclosure results in sustained expression of micro-Tuberin. The present disclosure thus provides methods of administering/delivering AAV which express micro-Tuberin to a mammalian subject, optionally a human being. These methods include transducing tissues (including, but not limited to, tissues such as muscle, organs such as liver and brain, and glands such as salivary glands) with one or more AAV of the present disclosure. Transduction may be carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et ah, Science, 251:761-766 (1991)], the myocyte-specific enhancer binding factor MEF-2 [Cserjesi and Olson, Mol Cell Biol 11: 4854-4862 (1991)], control elements derived from the human skeletal actin gene [Muscat et al, Mol Cell Biol, 7: 4089-4099 (1987)], the cardiac actin gene, muscle creatine kinase sequence elements [See Johnson et ah, Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et ah, Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

In some embodiments, delivering the vector of the instant disclosure to a cell reduces mTOR signaling within the cell.

### Cells and Compositions

Another aspect of the disclosure is directed to a cell comprising the engineered micro-Tuberin disclosed herein, or the polynucleotide disclosed herein, or the vector disclosed herein. In some embodiments, the cell is a mammalian cell. In some embodiments, the cell is a human cell. In some embodiments, the cell is a brain cell, heart cell, kidney cell, skin cell, muscle cell or lung cell.

### Methods

Another aspect of the disclosure is directed to a method for delivering a micro-Tuberin polynucleotide or polypeptide to a cell comprising contacting the cell with the engineered micro-Tuberin disclosed herein, or the polynucleotide disclosed herein, or the vector disclosed herein. In some embodiments, the contacting is in vitro or in vivo.

In some embodiments, the cell is a mammalian cell, optionally a human cell.

Another aspect of the disclosure is directed to a method of treating a subject suffering from mTOR hyperactivation comprising administering to the subject the engineered micro-Tuberin disclosed herein, or the polynucleotide disclosed herein, or the vector disclosed herein. In some embodiments, the contacting is in vitro or in vivo.

In some embodiments, the disease is selected from the group consisting of tuberous sclerosis complex (TSC) type 2, focal cortical dysplasia type 2, lymphangioleiomyomatosis, and renal angiomyolipoma.

In some embodiments, the administration is achieved systemically, intravascularly, intracerebrally, or intrathecally; or optionally the administration is into the renal artery or vein, or into the lungs.

In some embodiments, the administration is achieved by an AAV vector, and wherein the AAV vector comprises a capsid selected from AAV1, AAV2, AAV3, AAV4, AAV5, AAV6, AAV7, AAV8, AAV9, AAV10, AAV11, or AAV12 capsid. In some embodiments, the AAV vector is parenterally administered by injection, infusion or implantation.

Also described herein are methods of administering a therapeutically effective dose (or doses, administered essentially simultaneously or doses given at intervals) of AAV that encode micro-Tuberin to a subject in need thereof. In some embodiment, the subject is a mammal. In some embodiments, the subject is a human.

Methods of transducing a target cell with AAV, in vivo or in vitro, are contemplated by the disclosure. The term “transduction” is used to refer to the administration/delivery of a polynucleotide of interest (e.g., a polynucleotide sequence encoding micro-Tuberin) to a recipient cell either in vivo or in vitro, via a replication deficient AAV described resulting in expression of micro-Tuberin by the recipient cell.

Titers of AAV to be administered in methods of the disclosure will vary depending, for example, on the particular AAV, the mode of administration, the treatment goal, the individual, and the cell type(s) being targeted, and may be determined by methods standard in the art. Titers of AAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg).

A therapeutically effective dose of the AAV vector is in some embodiments a dose of AAV ranging in one or more administrations in ranges from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or lei 3 vg/kg to about 3e14 vg/kg, or about 1×13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg. The disclosure also comprises, or consists essentially of, or yet further consists of compositions comprising, or consisting essentially of, or yet further consisting of these ranges of AAV vector.

For example, a therapeutically effective amount of AAV vector is a dose of 1e13 vg/kg, about 2e13 vg/kg, about 3e13 vg/kg, about 4e13 vg/kg, about 5e13 vg/kg, about 6e13 vg/kg, about 7e13 vg/kg, about 8e13 vg/kg, about 9e13 vg/kg, about 1e14 vg/kg, about 2e14 vg/kg, about 3e14 vg/kg, about 4e14 vg/kg and 5e14 vg/kg. The disclosure also comprises, or consists essentially of, or yet further consists of compositions comprising, or consisting essentially of, or yet further consisting of these doses of AAV vector.

A therapeutic effective amount of AAV is in some embodiments a dose of AAV ranging from about 1e14 vg/kg to about 1e15 vg/kg or about 1e15 vg/kg to about 1e16 vg/kg. In some embodiments, the disclosure provides methods of administering an AAV vector of the disclosure to subject at a dose of about 1e14 vg/kg, about 1.5e14 vg/kg, about 2e14 vg/kg, about 2.5e14 vg/kg, about 3e14 vg/kg, about 3.5e14 vg/kg, about 4e14 vg/kg, about 4.5e14 vg/kg, about 5e14 vg/kg, about 5.5e14 vg/kg, about 6e14 vg/kg, about 6.5e14 vg/kg, about 7e14 vg/kg, about 7.5e14 vg/kg, about 8e14 vg/kg, about 8.5e14 vg/kg, about 9e14 vg/kg, about 9.5e14 vg/kg, about 1e15 vg/kg, about 1.5e15 vg/kg, about 2e15 vg/kg, about 2.5e15 vg/kg, about 3e15 vg/kg, about 3.5e15 vg/kg, about 4e15 vg/kg, about 4.5e15 vg/kg, or about 5e15 vg/kg. In some embodiments, the disclosure provides methods of administering an AAV vector of the disclosure to subject at a total dose of about 4.0e14 vg/kg, about 4.1e14 vg/kg, about 4.2e14 vg/kg, about 4.3e14 vg/kg, about 4.4e14 vg/kg, about 4.5e14 vg/kg, about 4.6e14 vg/kg, about 4.7e14 vg/kg, about 4.8e14 vg/kg, about 4.9e14 vg/kg, about 5.0e14 vg/kg, about 5.1e14 vg/kg, about 5.2e14 vg/kg, about 5.3e14 vg/kg, about 5.4e14 vg/kg, about 5.5e14 vg/kg, about 5.6e14 vg/kg, about 5.7e14 vg/kg, about 5.8e14 vg/kg, about 5.9e14 vg/kg, or about 6e14 vg/kg.

In various embodiments, the administering step may comprise administering the total dose in 1, 2, 3, 4, 5, 6, 7, 8, 9, 10 or more divided doses. For examples, the total dose may be delivered by injection to multiple sites on the subject or to the subject spaced over several minutes, several hours, or several days.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the AAV (in particular, the AAV ITRs and capsid protein) of the disclosure may be chosen and/or matched by those skilled in the art taking into account the infection and/or disease state being treated and the target cells/tissue(s) that are to express the micro-Tuberin.

The disclosure provides for local administration and systemic administration of an effective dose of AAV and compositions of the disclosure. For example, systemic administration is administration into the circulatory system so that the entire body is affected. Systemic administration includes enteral administration such as absorption through the gastrointestinal tract and parental administration through injection, infusion or implantation.

In particular, actual administration of AAV of the present disclosure may be accomplished by using any physical method that will transport the AAV recombinant vector into the target tissue of an animal. Administration according to the disclosure includes, but is not limited to, injection into muscle, the bloodstream and/or directly into the liver. Simply resuspending an AAV in phosphate buffered saline has been demonstrated to be sufficient to provide a vehicle useful for muscle tissue expression, and there are no known restrictions on the carriers or other components that can be co-administered with the AAV (although compositions that degrade DNA should be avoided in the normal manner with AAV).

In some embodiments, the method of treating a subject comprises further administering an mTOR inhibitor to the subject. In some embodiments, the mTOR inhibitor is administered before the engineered micro-Tuberin disclosed herein, or the polynucleotide disclosed herein, or the vector disclosed herein. In some embodiments, the mTOR inhibitor is administered after the engineered micro-Tuberin disclosed herein, or the polynucleotide disclosed herein, or the vector disclosed herein. In some embodiments, the mTOR inhibitor is administered simultaneously (at the same time) with the engineered micro-Tuberin disclosed herein, or the polynucleotide disclosed herein, or the vector disclosed herein.

In some embodiments, the mTOR inhibitor is selected from rapamycin, pimecrolimus, tacrolimus, sirolimus, temsirolimus, everolimus, ridaforolimus, samimod, AZD8055, PF04691502, SF1126, XL765, or pharmaceutically acceptable salts and combinations thereof. In some embodiments, the mTOR inhibitor is rapamycin or everolimus.

In some embodiments, the subject is a mammal. In some embodiments, the subject is a human.

Further provided herein are kits comprising, or consisting essentially of, or yet further consisting of, any of one or more of the embodiments disclosed herein and optional instructions for use. The kits can comprise, or consist essentially of, or yet further consist of one or more of the compositions disclosed herein and a corticosteroid or one or more of the combination therapy provided herein and optional instructions for use.

### EXAMPLES

### Example 1: Materials and Methods

Construction of MT candidates based on rational design and AI protein structure prediction: The native full-length sequence of human Tuberin isoform 1 (NP_000539.2-SEQ ID NO: 1) was used as the input for Alphafold 2, generating a structural model based only on sequence. This structure was overlaid on one of the monomers of tuberin in the cryoEM structure of the TSC complex (PDBid: 7DL2). Next, the mutations known to cause tuberous sclerosis as well as the known phosphorylation sites were mapped to the structure. It was noted that all of the mutations known to cause disease were contained within the structured regions of the protein whereas many of the phosphorylation sites were found within the unstructured regions. Therefore, the initial approach to truncating the sequence was to remove the unstructured regions and replace them with short flexible linkers (consisting of glycine and serine residues). The length of the flexible linkers was always determined by measuring the intervening distance in the structure and incorporating sufficient residues to span that distance based on a per residue span of 3.5 Å/residue.

Because the structured region of the carboxy-terminus (C-terminus) contains functional components of Tuberin and/or components critical for TSC complex formation such as the GTPase-activating protein (GAP) domain as well as the tuberin dimerization domain and GAP extension, this region (with the unstructured regions removed) was common to all of the MT candidates. This common region (CR) corresponds to residues 915-923 of SEQ ID NO: 1 (consisting of the GAP extension regions) with a 3 glycine-serine (GS) residue linker engineered to bridge this region with a Tuberin dimerization interface at residues 1016-1081 of SEQ ID NO: 1. This Tuberin dimerization interface was linked to a second Tuberin dimerization domain corresponding to residues 1183-1215 of SEQ ID NO: 1 with another 5 GS residue linker. Lastly, the second Tuberin dimerization domain was bridged to residues 1517-1758 (the GAP domain residues) of SEQ ID NO: 1 with an additional 3 GS residue linker. Regions corresponding to the hamartin interfaces and/or other domains in the amino-terminus (N-terminus) varied in configuration among MT candidates according to the design strategy/rationale described below. In cases when a naturally occurring methionine was not present in the beginning of the engineered micro-Tuberin (MT) construct, an ATG start codon was engineered.

MT2: MT2 was engineered to retain the N-terminal domain of Tuberin initiating at residue 94 and extending until residue 575 (residue numbers relative to human Tuberin isoform 1 (NP_000539.2)). A 12 GS residue flexible linker was engineered to bridge this region with the CR resulting in a construct of 984 residues.

MT3: MT3 was engineered to retain the N-terminal domain of Tuberin initiating at residue 94 and extending until residue 374 (removing a couple of the hamartin interfaces from the N-terminus). This region was then coupled to a region corresponding to residues 580-646 with a 17 residue GS linker. This region was then linked to a region corresponding to residues 686-923 with a 3 residue GS linker (effectively increasing the number of hamartin interfaces adjacent to the GAP extensions which are a part of the CR). This resulted in a construct of 986 residues.

MT5: Both MT2 and MT3 lack some of the hamartin interfacial regions that contain residues which when mutated are associated with tuberous sclerosis, therefore MT5 was engineered to contain all of the hamartin interfacial regions that contain residues which when mutated are associated with tuberous sclerosis. MT5 was engineered to retain the N-terminal domain of Tuberin initiating at residue 94 and extending until residue 188. This region was joined to residues 230-267 with a 3 residue GS linker. This region was then linked to 355-419 with an 8 GS residue linker, which was then linked to residues 459-520 with a 4 GS residue linker. This region was then linked to residues 580-646 with a 3 GS residue linker. This region was then linked to a region corresponding to residues 686-923 with a 3 residue GS linker (again directly connecting into the GAP extensions in the CR) resulting in a construct of 966 residues.

MT6: MT6 is synonymous to MT3 but contains an additional 44 residues on the N-terminus (initiating at residue 50 rather than 94 in MT3) yielding a construct of 1029 residues.

MT7: MT7 was designed to extend the structured region of the C-terminus as far into the N-terminal hamartin interfaces as possible (beginning with residue 271) yielding a 997 residue construct.

MT8: MT8 was designed similarly to MT3 but includes residues 72-435 with a 9 residue linker to 577 at the C-terminus resulting in a 1064 residue construct.

MT9: MT9 was designed to contain all of the structured regions encoded within full-length Tuberin resulting in a 1266 residue construct. MT9 was engineered to retain the full structured N-terminal domain of Tuberin initiating at residue 1 and extending until residue 646. This region was then linked to a region corresponding to residues 686-923 (which proceeds into the CR) with a 3 residue GS linker.

To accommodate for this larger protein MT9 construct of 1266 residues, which corresponds to a 3.8 kilobase pair complementary DNA sequence and that can fit within the constraints of a 4.7 kilobase AAV vector, Applicant swapped out the CBA promoter with a truncated EFS promoter in addition to including the SV40 intron that increases transgene expression. Additionally, the 225 base pair fragment of the TSC2 3′UTR was excluded in the MT8 and MT9-AAV9 vector in order not to exceed the 4.7 kilobase AAV vector capacity.

Next, the finalized sequences were subjected to homology modeling in SwissModel using 7DL2 as a template to ensure that the inserted flexible linkers were sufficient to span the gaps allowing the structural elements to align. Finally, the sequences were used as the input for Alphafold 2 generating structural models for each based only on sequence. The resulting homology models and Alphafold generated structures were aligned with one of the tuberin monomers in 7DL2 to assess the similarities and differences.

Functional efficacy testing of MT candidates to inhibit phosphorylation of S6K: Protein sequences for Micro-Tuberin and cTuberin constructs were reverse translated and codon optimized in order to enhance mRNA stability and to maximize transcriptional and translational efficiency. All Tuberin constructs were gene synthesized by VectorBuilder.com. For transient transfections, HEK293 cells were cultured at 37° C. with 5% CO2 in Dulbecco's modified Eagle's medium (DMEM) with 10% fetal bovine serum (FBS). Cells were transiently transfected in a 24-well plate format at 80% confluency using Lipofectamine™ 3000 according to the manufacturer's protocol (Thermofisher). An equivalent molar concentration of MT candidates, cTuberin, full-length WT Tuberin, and eGFP mock vector control were used for transfections, which were performed in duplicate biological replicates and co-transfected with a constant amount of plasmids encoding TSC1 and S6K. Cells were allowed to culture for 48 hours prior to harvesting cell pellets, which were stored at −80° C. prior to performing an ELISA for phosphorylated and total S6K. As a positive control, HEK293 cells were treated with the PI3K/Akt signaling pathway inhibitor, wortmannin, at a final concentration of 1 μM for 30 minutes prior to cell harvest. To quantify for total S6K and phosphorylated-S6K (at position T389) levels, the p70 S6K (Total/Phospho) InstantOne ELISA™ kit was used according to the manufacturer's protocol (ThermoFisher). Quantification of phosphorylated S6K1 (position T389) levels were normalized to total S6K1 levels in all experimental and control conditions. All data from two biological transfection replicates and three ELISA technical replicates were calculated as the mean±SD. Data was also analyzed as a percentage of activity relative to wild type (WT) or full-length Tuberin. Statistical analyses were performed using one-way ANOVA with Tukey's multiple comparisons test.

Functional efficacy testing of AAV9 MT candidates to inhibit phosphorylation of S6 in TSC2 knockout astrocytes: A CRISPR/Cas9 non-homology end joining (NHEJ) strategy was used to disrupt both TSC2 alleles in a normal human induced pluripotent stem cell (iPSC) line using multi-small guide RNAs, which were designed to target introns 1 and 4 and delete an approximate 4.9 kilobase pair fragment encompassing exons 2, 3, and 4. Excision of exons 2-4 results in a frameshift and a subsequent premature stop codon at c.542-544 causing nonsense mediated decay. Western blot analysis confirmed loss of Tuberin protein in TSC2 knockout (KO) iPSCs and increased mTORC1 signaling as demonstrated by increased pS6K1 levels. TSC2 KO iPSCs were then differentiated into astrocytes and purified using magnetic-activated cell sorting (MACS®) technology using the Anti-GLAST (ACSA-1) MicroBead Kit (Miltenyi-Biotec). Astrocytes were plated at a density of 25,000 cells per well of a 96 well tissue culture plate that was previously coated with poly-ornithine and laminin to promote cell attachment. Micro-Tuberins, cTuberin, and mock GFP AAV9 vectors were packaged into AAV9 viral particles and then used to infect TSC2 KO astrocytes in quadruplicate experimental wells at a multiplicity of infection (MOI) of 20,000. Infected astrocytes were allowed to culture for 7 days and then were processed for immunofluorescence analysis and assessed for phosphor-S6 levels (at position Serine 235/236, Cell Signaling Technologies #2211). DAPI was used to stain nuclei. Fluorescent images were captured from four biological replicate wells using an EVOS microscope (Thermofisher). ImageJ was used to analyze images and calculate fluorescence intensity and pS6 area occupancy in control and experimental groups. Statistical analyses were performed using one-way ANOVA with Tukey's multiple comparisons test.

### Example 2

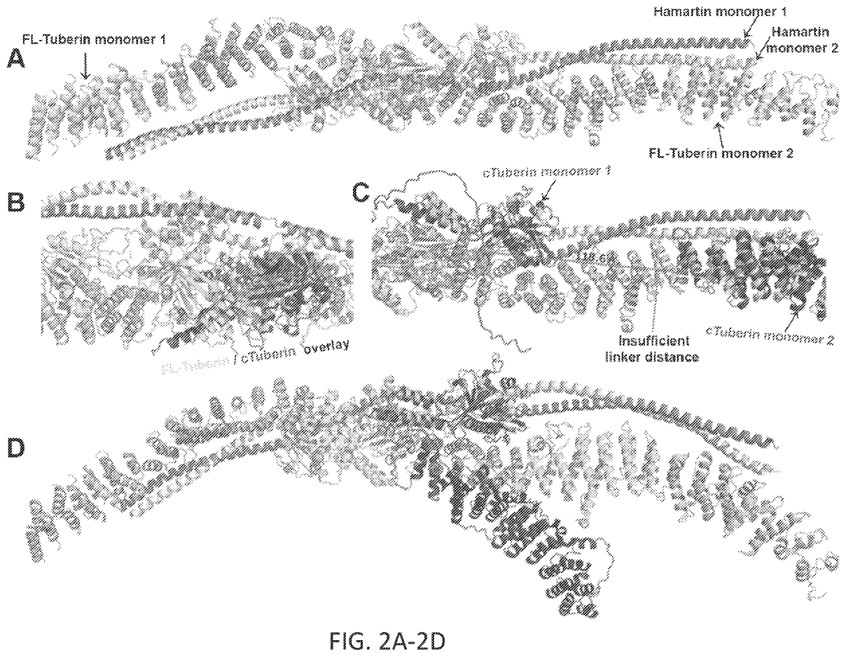

Recently, a condensed form of Tuberin (cTuberin) was designed and evaluated as a potential gene therapy candidate for TSC type 2 (Cheah, P. S. et al; Sci Adv 7, doi:10.1126/sciadv.abb1703 (2021)). Although cTuberin was engineered to contain two important functional domains (Hamartin binding and GAP domain), it lacks other critical domains important for proper self-dimerization and physiological interactions to other components within the full TSC complex. Using Alphafold 2, the Applicant's analysis confirmed cTuberin lacks critical structural homology to that of full-length Tuberin (FIGS. 2A-2D), which prevents physiological dimerization and proper TSC complex formation. This fundamental design limitation is consistent with recent data demonstrating c-Tuberin exhibits limited improvement in motor coordination, partial functional activity in vivo (by the ability to reduce pS6 levels), and limited survival benefit in a stochastic Tsc2 cKO mouse model. Further, whether cTuberin shows efficacy in preventing or mitigating seizures in this mouse model was not previously evaluated. All of this data collectively suggests that the partial efficacy demonstrated by cTuberin is most likely due to a lack of structural homology to that of the full-length Tuberin protein, rendering it with reduced functional capacity.

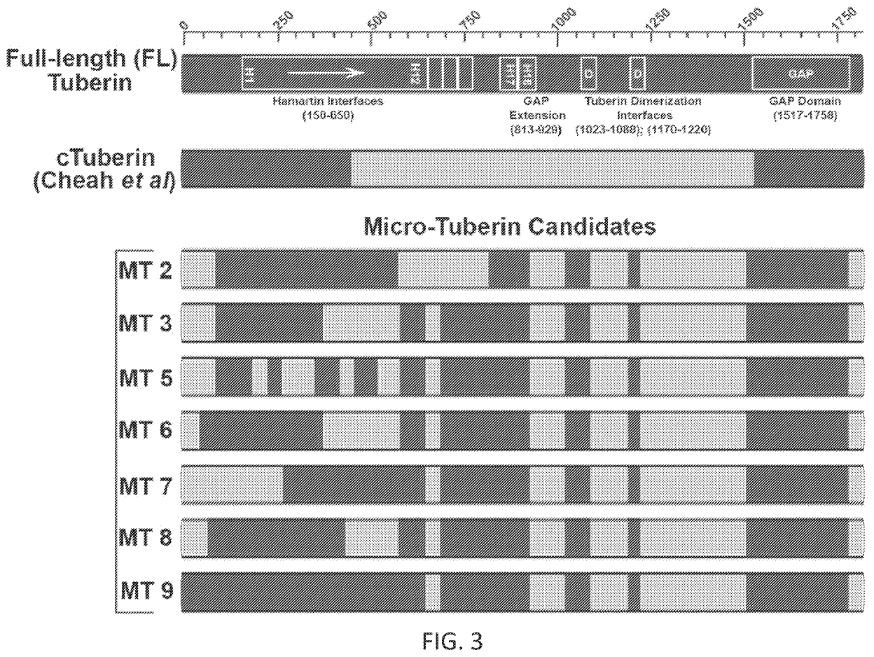

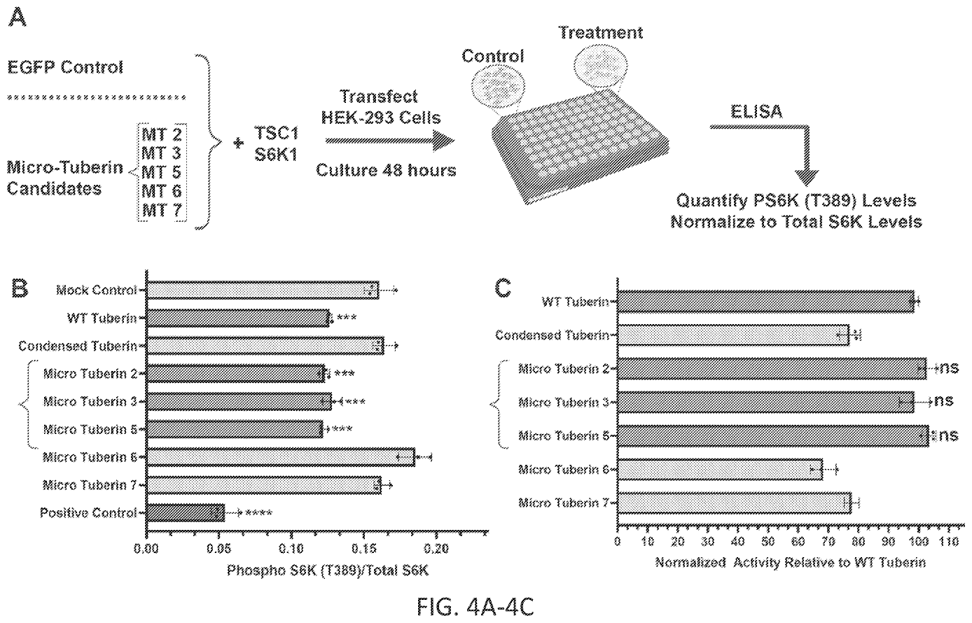

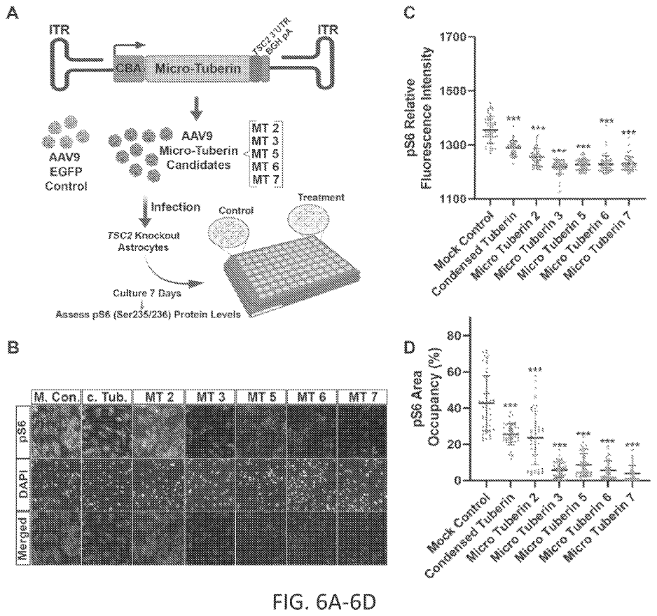

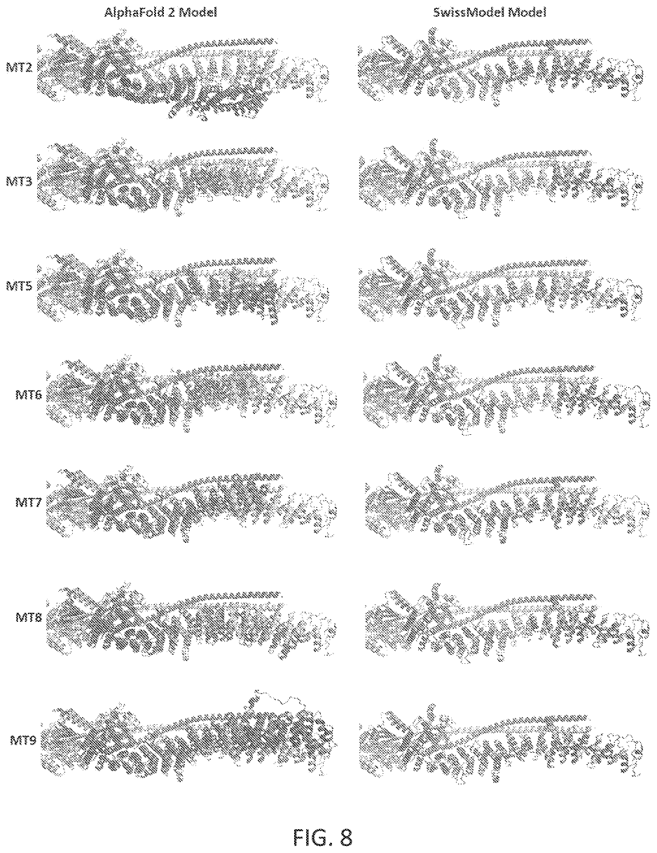

To this end, Applicant leveraged principles based on rational protein design and artificial-intelligence-based protein structure prediction in order to develop micro-Tuberin candidates with increased structural similarity and enhanced functional capacity comparable to that of full-length Tuberin (FIG. 3). Micro-Tuberin candidates were designed by removing unstructured regions based on a recent cryo-EM structure (Yang, Huirong, et al. “Structural insights into TSC complex assembly and GAP activity on Rheb.” Nature communications 12.1 (2021): 339) as well as the structure predicted using Alphafold 2, a highly accurate, open-source, protein structure prediction software tool. The remaining structured regions were then reconnected using the appropriate number of flexible residues (Gly and Ser) to span the distances calculated from the structures using Pymol. Finally, the construct size was further reduced to fit within the limitations of the AAV vectors by removing different portions of the structured regions that were predicted to be least likely to affect micro-Tuberin function. Based on these principles, seven novel micro-Tuberin candidates were developed (FIG. 3), which were validated by homology modeling using Swissmodel and folding prediction using Alphafold 2 (FIG. 8). The final constructs were gene synthesized, packaged them into AAV9 viral vectors, and tested for their ability to suppress mTORC1 signaling using multiple functional in vitro assays (FIGS. 4A-4C, 5A-5E, and 6A-6D). In sum, novel micro-Tuberins of the instant disclosure are viable candidates for gene replacement therapy for treating TSC type 2.

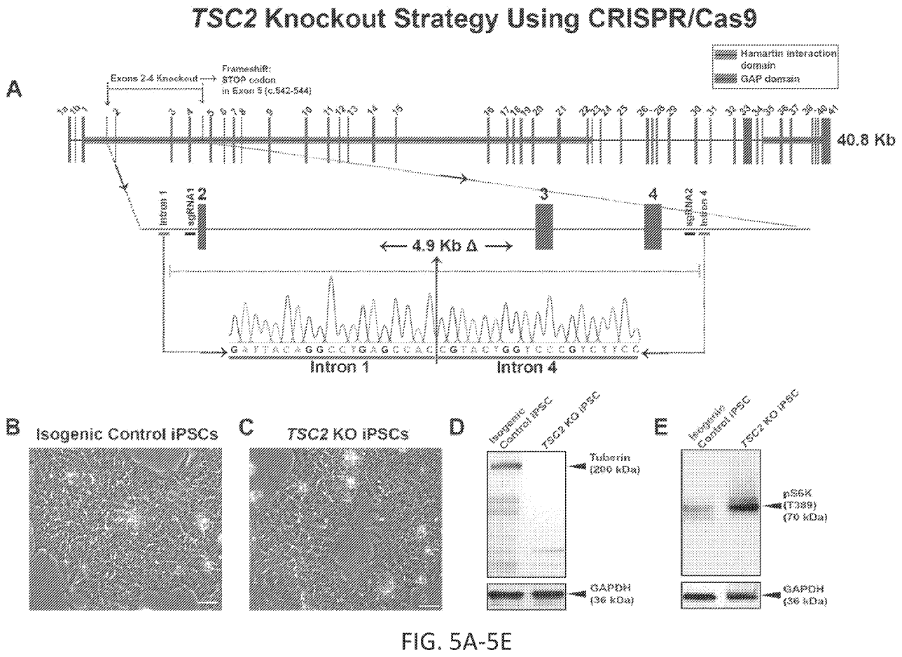

Next, Applicant engineered seven micro-Tuberin candidates within a recombinant AAV vector genome. Five of the micro-Tuberin candidates: MT2, MT3, MT5, MT6, and MT7, contain the same regulatory and vector backbone sequences (FIG. 5). The expression of each of these micro-Tuberin constructs is controlled by the strong CAG promoter (850 base pairs), which is a hybrid promoter consisting of the CMV early enhancer joined to the chicken β-actin promoter and SV40 intron. Additionally, to further enhance expression levels, all micro-Tuberin cDNAs have been codon optimized and include a Kozak translation initiation sequence, which promotes translation initiation of the ATG start codon. To allow for efficient transcription termination and polyadenylation, a hybrid 3′ untranslatable region (UTR) was engineered by joining an endogenous TSC2 sequence of 225 base pairs immediately downstream of the TGA STOP codon with the Bovine growth Hormone (BGH) polyadenylation signal (208 base pairs). MT8 contains all of the regulatory sequences described in the preceding section except for the inclusion of the 225 base pair TSC2 3′UTR. MT9 contains a truncated EFS promoter in addition to the SV40 intron (309 base pairs) to drive transgene expression. Both MT8 and MT9 have been codon optimized and include a Kozak translation initiation sequence, which promotes translation initiation of the ATG start codon. In addition, both MT8 and MT9 contain the BGH polyadenylation signal (208 base pairs).

### LIST OF SEQUENCES

SEQ ID NO: 1, Protein, Homo Sapiens, Tuberin isoform 1 wild type full length
sequence
MAKPTSKDSGLKEKFKILLGLGTPRPNPRSAEGKQTEFIITAEILRELSMECGLNNRIR
MIGQICEVAKTKKFEEHAVEALWKAVADLLQPERPLEARHAVLALLKAIVQGQGER
LGVLRALFFKVIKDYPSNEDLHERLEVFKALTDNGRHITYLEEELADFVLQWMDVG
LSSEFLLVLVNLVKFNSCYLDEYIARMVQMICLLCVRTASSVDIEVSLQVLDAVVCY
NCLPAESLPLFIVTLCRTINVKELCEPCWKLMRNLLGTHLGHSAIYNMCHLMEDRAY
MEDAPLLRGAVFFVGMALWGAHRLYSLRNSPTSVLPSFYQAMACPNEVVSYEIVLSI
TRLIKKYRKELQVVAWDILLNIIERLLQQLQTLDSPELRTIVHDLLTTVEELCDQNEFH
GSQERYFELVERCADQRPESSLLNLISYRAQSIHPAKDGWIQNLQALMERFFRSESRG
AVRIKVLDVLSFVLLINRQFYEEELINSVVISQLSHIPEDKDHQVRKLATQLLVDLAEG
CHTHHFNSLLDIIEKVMARSLSPPPELEERDVAAYSASLEDVKTAVLGLLVILQTKLY
TLPASHATRVYEMLVSHIQLHYKHSYTLPIASSIRLQAFDFLLLLRADSLHRLGLPNK
DGVVRFSPYCVCDYMEPERGSEKKTSGPLSPPTGPPGPAPAGPAVRLGSVPYSLLFRV
LLQCLKQESDWKVLKLVLGRLPESLRYKVLIFTSPCSVDQLCSALCSMLSGPKTLERL
RGAPEGFSRTDLHLAVVPVLTALISYHNYLDKTKQREMVYCLEQGLIHRCASQCVV
ALSICSVEMPDIIIKALPVLVVKLTHISATASMAVPLLEFLSTLARLPHLYRNFAAEQY
ASVFAISLPYTNPSKFNQYIVCLAHHVIAMWFIRCRLPFRKDFVPFITKGLRSNVLLSF
DDTPEKDSFRARSTSLNERPKSLRIARPPKQGLNNSPPVKEFKESSAAEAFRCRSISVS
EHVVRSRIQTSLTSASLGSADENSVAQADDSLKNLHLELTETCLDMMARYVFSNFTA
VPKRSPVGEFLLAGGRTKTWLVGNKLVTVTTSVGTGTRSLLGLDSGELQSGPESSSS
PGVHVRQTKEAPAKLESQAGQQVSRGARDRVRSMSGGHGLRVGALDVPASQFLGS
ATSPGPRTAPAAKPEKASAGTRVPVQEKTNLAAYVPLLTQGWAEILVRRPTGNTSW
LMSLENPLSPFSSDINNMPLQELSNALMAAERFKEHRDTALYKSLSVPAASTAKPPPL
PRSNTVASFSSLYQSSCQGQLHRSVSWADSAVVMEEGSPGEVPVLVEPPGLEDVEAA
LGMDRRTDAYSRSSSVSSQEEKSLHAEELVGRGIPIERVVSSEGGRPSVDLSFQPSQPL
SKSSSSPELQTLQDILGDPGDKADVGRLSPEVKARSQSGTLDGESAAWSASGEDSRG
QPEGPLPSSSPRSPSGLRPRGYTISDSAPSRRGKRVERDALKSRATASNAEKVPGINPS
FVFLQLYHSPFFGDESNKPILLPNESQSFERSVQLLDQIPSYDTHKIAVLYVGEGQSNS
ELAILSNEHGSYRYTEFLTGLGRLIELKDCQPDKVYLGGLDVCGEDGQFTYCWHDDI
MQAVFHIATLMPTKDVDKHRCDKKRHLGNDFVSIVYNDSGEDFKLGTIKGQFNFVH
VIVTPLDYECNLVSLQCRKDMEGLVDTSVAKIVSDRNLPFVARQMALHANMASQV
HHSRSNPTDIYPSKWIARLRHIKRLRQRICEEAAYSNPSLPLVHPPSHSKAPAQTPAEP
TPGYEVGQRKRLISSVEDFTEFV
SEQ ID NO: 2, DNA, Artificial sequence, Inverted terminal repeat (ITR) sequence
Ctgcgcgctcgctcgctcactgaggccgcccgggcaaagcccgggcgtcgggcgacctttggtcgcccggcctcagtgagcgag
cgagcgcgcagagagggagtggccaactccatcactaggggttcct
SEQ ID NO: 3, DNA, Artificial sequence, Chicken Beta-Actin (CBA) Promoter-
cgttacataacttacggtaaatggcccgcctggctgaccgcccaacgacccccgcccattgacgtcaataatgacgtatgttcccatag
taacgccaatagggactttccattgacgtcaatgggtggagtatttacggtaaactgcccacttggcagtacatcaagtgtatcatatgcc
aagtacgccccctattgacgtcaatgacggtaaatggcccgcctggcattatgcccagtacatgaccttatgggactttcctacttggca
gtacatctactcgaggccacgttctgcttcactctccccatctcccccccctccccacccccaattttgtatttatttattttttaattatttt
gtgcagcgatgggggcggggggggggggggggcgcgcgccaggcggggcggggcggggcgaggggcggggcggggcgaggc
ggagaggtgcggcggcagccaatcagagcggcgcgctccgaaagtttccttttatggcgaggcggcggcggcggcggccctataa
aaagcgaagcgcgcggcgggcgggagcgggatcagccaccgcggtggcggcctagagtcgacgaggaactgaaaaaccagaa
agttaactggtaagtttagtctttttgtcttttatttcaggtcccggatccggtggtggtgcaaatcaaagaactgctcctcagtggatgttgc
ctttacttctaggcctgtacggaagtgttacttctgctctaaaagctgeggaattgtacccgcggccgatccaccggtccggaattcccg
ggatatcgtcgacccacgcgtccgggccccacgctgcgcacccgcgggtttgct
SEQ ID NO: 4, DNA, Artificial sequence, TSC2 3′ UTR sequence
ggccggggccctccctcctgcactggccttggacggtattgcctgtcagtgaaataaataaagtcctgaccccagtgcacagacatag
aggcacagattgcagtcagacagctcttttattgactttgtctgcttggtgcgggggttgggggggtgtcgaggctctagaagcggcca
tgcccacagaagtggtacacagaagcaggcacagccagctccgagggc
SEQ ID NO: 5, DNA, Artificial sequence, Bovine growth hormone (BGH) poly A
sequence
acccagctttcttgtacaaagtgggaattcctagagctcgctgatcagcctcgactgtgccttctagttgccagccatctgttgtttgcccct
cccccgtgccttccttgaccctggaaggtgccactcccactgtcctttcctaataaaatgaggaaattgcatcgcattgtctgagtaggtg
tcattctattctggggggtggggtggggcaggacagcaagggggaggattgggaagagaatagcaggcatgctggggagggccg
c
SEQ ID NO: 6, DNA, Artificial sequence, 3′ ITR-Inverted terminal repeat (ITR)
sequence
aggaacccctagtgatggagttggccactccctctctgcgcgctcgctcgctcactgaggccgggcgaccaaaggtcgcccgacgc
ccgggctttgcccgggcggcctcagtgagcgagcgagcgcgcag
SEQ ID NO: 7, DNA, Artificial sequence, Micro-Tuberin 2 coding sequence
atgccgctggaggcccggcacgcggtgctggctctgctgaaggccatcgtgcaggggcagggcgagcgtttgggggtcctcagag
ccctcttctttaaggtcatcaaggattacccttccaacgaagaccttcacgaaaggctggaggttttcaaggccctcacagacaatggga
gacacatcacctacttggaggaagagctggctgactttgtcctgcagtggatggatgttggcttgtcctcggaattccttctggtgctggt
gaacttggtcaaattcaatagctgttacctcgacgagtacatcgcaaggatggttcagatgatctgtctgctgtgcgtccggaccgcgtc
ctctgtggacatagaggtctccctgcaggtgctggacgccgtggtctgctacaactgcctgccggctgagagcctcccgctgttcatcg
ttaccctctgtcgcaccatcaacgtcaaggagctctgcgagccttgctggaagctgatgcggaacctccttggcacccacctgggcca
cagcgccatctacaacatgtgccacctcatggaggacagagcctacatggaggacgcgcccctgctgagaggagccgtgttttttgtg
ggcatggctctctggggagcccaccggctctattctctcaggaactcgccgacatctgtgttgccatcattttaccaggccatggcatgt
ccgaacgaggtggtgtcctatgagatcgtcctgtccatcaccaggctcatcaagaagtataggaaggagctccaggtggtggcgtgg
gacattctgctgaacatcatcgaacggctccttcagcagctccagaccttggacagcccggagctcaggaccatcgtccatgacctgtt
gaccacggtggaggagctgtgtgaccagaacgagttccacgggtctcaggagagatactttgaactggtggagagatgtgcggacc
agaggcctgagtcctccctcctgaacctgatctcctatagagcgcagtccatccacccggccaaggacggctggattcagaacctgca
ggcgctgatggagagattcttcaggagcgagtcccgaggcgccgtgcgcatcaaggtgctggacgtgctgtcctttgtgctgctcatc
aacaggcagttctatgaggaggagctgattaactcagtggtcatctcgcagctctcccacatccccgaggataaagaccaccaggtcc
gaaagctggccacccagttgctggtggacctggcagagggctgccacacacaccacttcaacagcctgctggacatcatcgagaag
gtgatggcccgctccctctccccacccccggagctggaagaaagggatgtggccgcatactcggcctccttggaggatgtgaagac
agccgtcctggggcttctggtcatccttcagaccaagctgggcagttcgggcagctccgggtcctccggctcctctatgcctgacatc
atcatcaaggcgctgcctgttctggtggtgaagctcacgcacatctcagccacagccagcatggccgtcccactgctggagttcctgtc
cactctggccaggctgccgcacctctacaggaactttgccgcggagcagtatgccagtgtgttcgccatctccctgccgtacaccaac
ccctccaagtttaatcagtacatcgtgtgtctggcccatcacgtcatagccatgtggttcatcaggtgccgcctgcccttccggaaggatt
ttgtccctttcatcactaagggcctgcggtccaatgtcctcttgtctggatccggaaaaaacctccacctggagctcacggaaacctgtc
tggacatgatggctcgatacgtcttctccaacttcacggctgtcccgaagaggtctcctgtgggcgagttcctcctagcgggtggcagg
accaaaacctggctggttgggaacaagcttgtcactgtgacgacaagcgtgggaaccgggacccggtcgttactaggcggcagcgg
agggctggcggcctatgtgcccctgctgacccagggctgggcggagatcctggtccggaggcccacagggaacaccagctggct
gatgagcctggagaacccgggatctggtatcaaccccagtttcgtgttcctgcagctctaccattcccccttctttggcgacgagtcaaa
caagccaatcctgctgcccaatgagtcacagtcctttgagcggtcggtgcagctcctcgaccagatcccatcatacgacacccacaag
atcgccgtcctgtatgttggagaaggccagagcaacagcgagctcgccatcctgtccaatgagcatggctcctacaggtacacggag
ttcctgacgggcctgggccggctcatcgagctgaaggactgccagccggacaaggtgtacctgggaggcctggacgtgtgtggtga
ggacggccagttcacctactgctggcacgatgacatcatgcaagccgtcttccacatcgccaccctgatgcccaccaaggacgtgga
caagcaccgctgcgacaagaagcgccacctgggcaacgactttgtgtccattgtctacaatgactccggtgaggacttcaagcttggc
accatcaagggccagttcaactttgtccacgtgatcgtcaccccgctggactacgagtgcaacctggtgtccctgcagtgcaggaaag
acatggagggccttgtggacaccagcgtggccaagatcgtgtctgaccgcaacctgcccttcgtggcccgccagatggccctgcac
gcaaatatggcctcacaggtgcatcatagccgctccaaccccaccgatatctacccctccaagtggattgcccggctccgccacatca
agcggctccgccagcggatctgcgaggaagccgcctactccaaccccagctga
SEQ ID NO: 8, DNA, Artificial sequence, Micro-Tuberin 2 codon optimized sequence
agagccctgttcttcaaggtgatcaaggactacccaagcaatgaagacctgcatgagcgcctggaggtgttcaaagccctgaccgata
atggccggcacatcacctacctggaggaggaactggcagactttgtcctgcaatggatggatgtggggctgagctctgagtttctgctg
gtgctggtgaatctggtgaagtttaacagctgctacctggatgagtatatcgccaggatggtgcagatgatttgcctgctgtgtgtgcgca
ccgccagctcagtggacattgaagtgtctctgcaggtgctggatgccgtggtgtgctacaactgcctgcccgctgagtccctgcccctg
ttcatcgtgactctgtgccggaccatcaatgtcaaggagctgtgcgagccttgctggaaactgatgagaaatctgctgggcacacatct
ggggcacagcgctatctacaacatgtgccacctgatggaagatcgcgcttatatggaagacgcccctctgctgaggggagccgtgttc
ttcgtgggaatggccctgtggggagcccacaggctctactccctgcggaacagccccacctctgtgctgccgtccttctatcaggccat
ggcctgtcccaacgaggtggtgtcttacgagatagtgctgtccatcaccagactgattaagaaatacaggaaggagctgcaggtggtg
gcctgggatattctcctgaatatcatcgagcggctgctgcaacagctgcagaccctcgattctcctgagctgcggaccatcgtgcacga
cctgctgaccaccgtggaggaactgtgcgaccagaacgaattccacgggtctcaggagaggtacttcgagctcgttgagagatgtgc
tgatcagagacccgagtcctccctcctgaacctgattagctacagagcccagtccattcatccagctaaagatgggggattcagaacc
tgcaggcccttatggagcggttcttccggtcagagtcaagaggcgccgttcggattaaagtcctggacgtgctgagctttgtgctgctg
atcaatagacagttctatgaggaagagctgatcaactccgtggtgattagccagctgagccacatccccgaggacaaagaccaccag
gtgaggaagctggcgacacagctgctggtggacctggccgagggctgtcacacacatcacttcaacagcctcctggatatcatcgag
aaagtgatggcaaggtccctgagccccccccccgaactggaggaaagggacgtggccgcttactcagccagcctggaggacgtga
agactgccgtgctgggcctgctggtgattctgcagaccaagctcggcagttcgggcagctccgggtcctccggctcctctatgcccg
acattatcatcaaagccctgcctgtgctggtggtgaagctgacacacatcagcgccaccgcttcaatggccgtgcctctgctggaatttc
tgagcacactggcccggctgccccacctgtatcggaacttcgccgccgagcagtacgccagcgtgtttgccatcagcctgccttacac
aaacccctccaaatttaaccagtacatcgtctgcctggctcaccacgtgatcgccatgtggttcatccgatgcagactgccctttagaaa
ggacttcgtgcctttcatcaccaaggggctgaggagcaacgtgctgctgagcggatccggaaagaacctgcatctggagctgaccg
agacttgcctggacatgatggccagatacgtgttctccaatttcacagctgtgccaaagaggagccccgtgggcgagttcttgctggcc
ggaggcagaacaaagacctggctggtcggcaacaagcttgtgaccgtgaccactagcgtcggcaccggaaccagaagcctgctgg
gcggcagcggagggctcgccgcctatgtgccactgctgacgcaaggatgggccgagatcctggtccggaggccaaccggcaaca
catcctggctgatgtcactggagaatcccggatctggtatcaatccaagtttcgtgttcctgcagctgtaccacagccccttcttcggcg
acgagtccaataagcctatcctgctgcctaacgagtcccagagctttgagcgcagcgtgcaactgttggaccagatccccagctatga
cactcacaagatcgccgtgctgtatgtgggcgagggacagagcaactcagagctggccatcctgtccaacgagcacggcagctaca
gatacaccgagttcctgaccggcctgggccgcctgatcgagctgaaagactgccagccagataaggtgtatctgggcggcctggac
gtgtgcggcgaggacggacagtttacatactgctggcatgacgatattatgcaggccgtgttccacattgccacactgatgccaaccaa
agacgtggacaagcacaggtgcgataagaagagacacctgggtaatgacttcgtttcaatcgtgtacaatgatagcggcgaggacttc
aaactgggcaccatcaagggccaatttaattttgtgcacgtcatcgtgacacccctggactacgagtgcaacctcgtgtcactgcagtgt
cggaaggacatggaaggcctggtggatacctcagtggctaagattgtgtccgaccgcaacctgccattcgtggcccgacagatggcc
ctgcatgccaatatggccagccaggtgcaccattcacgctccaatcctactgatatctacccctctaagtggatcgccaggctgaggca
catcaagagactgcggcagcggatctgtgaggaggccgcctatagcaaccctagctga
SEQ ID NO: 9, DNA, Artificial sequence, Micro-Tuberin 2 Native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCGCTGGAGGCCCGGCACGCGGTGCTGGCTCTGCTGAAGGCCATC
GTGCAGGGGCAGGGCGAGCGTTTGGGGGTCCTCAGAGCCCTCTTCTTTAAGGTCA
TCAAGGATTACCCTTCCAACGAAGACCTTCACGAAAGGCTGGAGGTTTTCAAGG
CCCTCACAGACAATGGGAGACACATCACCTACTTGGAGGAAGAGCTGGCTGACT
TTGTCCTGCAGTGGATGGATGTTGGCTTGTCCTCGGAATTCCTTCTGGTGCTGGTG
AACTTGGTCAAATTCAATAGCTGTTACCTCGACGAGTACATCGCAAGGATGGTTC
AGATGATCTGTCTGCTGTGCGTCCGGACCGCGTCCTCTGTGGACATAGAGGTCTC
CCTGCAGGTGCTGGACGCCGTGGTCTGCTACAACTGCCTGCCGGCTGAGAGCCTC
CCGCTGTTCATCGTTACCCTCTGTCGCACCATCAACGTCAAGGAGCTCTGCGAGC
CTTGCTGGAAGCTGATGCGGAACCTCCTTGGCACCCACCTGGGCCACAGCGCCAT
CTACAACATGTGCCACCTCATGGAGGACAGAGCCTACATGGAGGACGCGCCCCT
GCTGAGAGGAGCCGTGTTTTTTGTGGGCATGGCTCTCTGGGGAGCCCACCGGCTC
TATTCTCTCAGGAACTCGCCGACATCTGTGTTGCCATCATTTTACCAGGCCATGG
CATGTCCGAACGAGGTGGTGTCCTATGAGATCGTCCTGTCCATCACCAGGCTCAT
CAAGAAGTATAGGAAGGAGCTCCAGGTGGTGGCGTGGGACATTCTGCTGAACAT
CATCGAACGGCTCCTTCAGCAGCTCCAGACCTTGGACAGCCCGGAGCTCAGGAC
CATCGTCCATGACCTGTTGACCACGGTGGAGGAGCTGTGTGACCAGAACGAGTT
CCACGGGTCTCAGGAGAGATACTTTGAACTGGTGGAGAGATGTGCGGACCAGAG
GCCTGAGTCCTCCCTCCTGAACCTGATCTCCTATAGAGCGCAGTCCATCCACCCG
GCCAAGGACGGCTGGATTCAGAACCTGCAGGCGCTGATGGAGAGATTCTTCAGG
AGCGAGTCCCGAGGCGCCGTGCGCATCAAGGTGCTGGACGTGCTGTCCTTTGTGC
TGCTCATCAACAGGCAGTTCTATGAGGAGGAGCTGATTAACTCAGTGGTCATCTC
GCAGCTCTCCCACATCCCCGAGGATAAAGACCACCAGGTCCGAAAGCTGGCCAC
CCAGTTGCTGGTGGACCTGGCAGAGGGCTGCCACACACACCACTTCAACAGCCT
GCTGGACATCATCGAGAAGGTGATGGCCCGCTCCCTCTCCCCACCCCCGGAGCTG
GAAGAAAGGGATGTGGCCGCATACTCGGCCTCCTTGGAGGATGTGAAGACAGCC
GTCCTGGGGCTTCTGGTCATCCTTCAGACCAAGCTGGGCAGTTCGGGCAGCTCC
GGGTCCTCCGGCTCCTCTATGCCTGACATCATCATCAAGGCGCTGCCTGTTCTG
GTGGTGAAGCTCACGCACATCTCAGCCACAGCCAGCATGGCCGTCCCACTGCTG
GAGTTCCTGTCCACTCTGGCCAGGCTGCCGCACCTCTACAGGAACTTTGCCGCGG
AGCAGTATGCCAGTGTGTTCGCCATCTCCCTGCCGTACACCAACCCCTCCAAGTT
TAATCAGTACATCGTGTGTCTGGCCCATCACGTCATAGCCATGTGGTTCATCAGG
TGCCGCCTGCCCTTCCGGAAGGATTTTGTCCCTTTCATCACTAAGGGCCTGCGGT
CCAATGTCCTCTTGTCTGGATCCGGAAAAAACCTCCACCTGGAGCTCACGGAAA
CCTGTCTGGACATGATGGCTCGATACGTCTTCTCCAACTTCACGGCTGTCCCGAA
GAGGTCTCCTGTGGGCGAGTTCCTCCTAGCGGGTGGCAGGACCAAAACCTGGCT
GGTTGGGAACAAGCTTGTCACTGTGACGACAAGCGTGGGAACCGGGACCCGGTC
GTTACTAGGCGGCAGCGGAGGGCTGGCGGCCTATGTGCCCCTGCTGACCCAGG
GCTGGGCGGAGATCCTGGTCCGGAGGCCCACAGGGAACACCAGCTGGCTGATGA
GCCTGGAGAACCCGGGATCTGGTATCAACCCCAGTTTCGTGTTCCTGCAGCTCT
ACCATTCCCCCTTCTTTGGCGACGAGTCAAACAAGCCAATCCTGCTGCCCAATGA
GTCACAGTCCTTTGAGCGGTCGGTGCAGCTCCTCGACCAGATCCCATCATACGAC
ACCCACAAGATCGCCGTCCTGTATGTTGGAGAAGGCCAGAGCAACAGCGAGCTC
GCCATCCTGTCCAATGAGCATGGCTCCTACAGGTACACGGAGTTCCTGACGGGCC
TGGGCCGGCTCATCGAGCTGAAGGACTGCCAGCCGGACAAGGTGTACCTGGGAG
GCCTGGACGTGTGTGGTGAGGACGGCCAGTTCACCTACTGCTGGCACGATGACA
TCATGCAAGCCGTCTTCCACATCGCCACCCTGATGCCCACCAAGGACGTGGACAA
GCACCGCTGCGACAAGAAGCGCCACCTGGGCAACGACTTTGTGTCCATTGTCTAC
AATGACTCCGGTGAGGACTTCAAGCTTGGCACCATCAAGGGCCAGTTCAACTTTG
TCCACGTGATCGTCACCCCGCTGGACTACGAGTGCAACCTGGTGTCCCTGCAGTG
CAGGAAAGACATGGAGGGCCTTGTGGACACCAGCGTGGCCAAGATCGTGTCTGA
CCGCAACCTGCCCTTCGTGGCCCGCCAGATGGCCCTGCACGCAAATATGGCCTCA
CAGGTGCATCATAGCCGCTCCAACCCCACCGATATCTACCCCTCCAAGTGGATTG
CCCGGCTCCGCCACATCAAGCGGCTCCGCCAGCGGATCTGCGAGGAAGCCGCCT
ACTCCAACCCCAGCTGAGGCCGGGGCCCTCCCTCCTGCACTGGCCTTGGACGGTA
TTGCCTGTCAGTGAAATAAATAAAGTCCTGACCCCAGTGCACAGACATAGAGGC
ACAGATTGCAGTCAGACAGCTCTTTTATTGACTTTGTCTGCTTGGTGCGGGGGTT
GGGGGGGTGTCGAGGCTCTAGAAGCGGCCATGCCCACAGAAGTGGTACACAGAA
GCAGGCACAGCCAGCTCCGAGGGCacccagctttcttgtacaaagtgggaattcctagagctcgctgatcagcc
tcgactgtgccttctagttgccagccatctgttgtttgcccctcccccgtgccttccttgaccctggaaggtgccactcccactgtcctttcc
taataaaatgaggaaattgcatcgcattgtctgagtaggtgtcattctattctggggggtggggtggggcaggacagcaagggggagg
attgggaagagaatagcaggcatgctggggagggccgcaggaacccctagtgatggagttggccactccctctctgcgcgctcgct
cgctcactgaggccgggcgaccaaaggtcgcccgacgcccgggctttgcccgggcggcctcagtgagcgagcgagcgcgcag
SEQ ID NO: 10, DNA, Artificial sequence, Micro-Tuberin 2 codon-optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCTCTGGAGGCTAGGCACGCCGTGCTGGCTCTGCTCAAGGCCATCG
TGCAGGGCCAGGGTGAGCGGCTGGGCGTGCTGAGAGCCCTGTTCTTCAAGGTGA
TCAAGGACTACCCAAGCAATGAAGACCTGCATGAGCGCCTGGAGGTGTTCAAAG
CCCTGACCGATAATGGCCGGCACATCACCTACCTGGAGGAGGAACTGGCAGACT
TTGTCCTGCAATGGATGGATGTGGGGCTGAGCTCTGAGTTTCTGCTGGTGCTGGT
GAATCTGGTGAAGTTTAACAGCTGCTACCTGGATGAGTATATCGCCAGGATGGTG
CAGATGATTTGCCTGCTGTGTGTGCGCACCGCCAGCTCAGTGGACATTGAAGTGT
CTCTGCAGGTGCTGGATGCCGTGGTGTGCTACAACTGCCTGCCCGCTGAGTCCCT
GCCCCTGTTCATCGTGACTCTGTGCCGGACCATCAATGTCAAGGAGCTGTGCGAG
CCTTGCTGGAAACTGATGAGAAATCTGCTGGGCACACATCTGGGGCACAGCGCT
ATCTACAACATGTGCCACCTGATGGAAGATCGCGCTTATATGGAAGACGCCCCTC
TGCTGAGGGGAGCCGTGTTCTTCGTGGGAATGGCCCTGTGGGGAGCCCACAGGC
TCTACTCCCTGCGGAACAGCCCCACCTCTGTGCTGCCGTCCTTCTATCAGGCCAT
GGCCTGTCCCAACGAGGTGGTGTCTTACGAGATAGTGCTGTCCATCACCAGACTG
ATTAAGAAATACAGGAAGGAGCTGCAGGTGGTGGCCTGGGATATTCTCCTGAAT
ATCATCGAGCGGCTGCTGCAACAGCTGCAGACCCTCGATTCTCCTGAGCTGCGGA
CCATCGTGCACGACCTGCTGACCACCGTGGAGGAACTGTGCGACCAGAACGAAT
TCCACGGGTCTCAGGAGAGGTACTTCGAGCTCGTTGAGAGATGTGCTGATCAGA
GACCCGAGTCCTCCCTCCTGAACCTGATTAGCTACAGAGCCCAGTCCATTCATCC
AGCTAAAGATGGGTGGATTCAGAACCTGCAGGCCCTTATGGAGCGGTTCTTCCG
GTCAGAGTCAAGAGGCGCCGTTCGGATTAAAGTCCTGGACGTGCTGAGCTTTGTG
CTGCTGATCAATAGACAGTTCTATGAGGAAGAGCTGATCAACTCCGTGGTGATTA
GCCAGCTGAGCCACATCCCCGAGGACAAAGACCACCAGGTGAGGAAGCTGGCG
ACACAGCTGCTGGTGGACCTGGCCGAGGGCTGTCACACACATCACTTCAACAGC
CTCCTGGATATCATCGAGAAAGTGATGGCAAGGTCCCTGAGCCCCCCCCCCGAA
CTGGAGGAAAGGGACGTGGCCGCTTACTCAGCCAGCCTGGAGGACGTGAAGACT
GCCGTGCTGGGCCTGCTGGTGATTCTGCAGACCAAGCTCGGCAGTTCGGGCAG
CTCCGGGTCCTCCGGCTCCTCTATGCCCGACATTATCATCAAAGCCCTGCCTGT
GCTGGTGGTGAAGCTGACACACATCAGCGCCACCGCTTCAATGGCCGTGCCTCTG
CTGGAATTTCTGAGCACACTGGCCCGGCTGCCCCACCTGTATCGGAACTTCGCCG
CCGAGCAGTACGCCAGCGTGTTTGCCATCAGCCTGCCTTACACAAACCCCTCCAA
ATTTAACCAGTACATCGTCTGCCTGGCTCACCACGTGATCGCCATGTGGTTCATC
CGATGCAGACTGCCCTTTAGAAAGGACTTCGTGCCTTTCATCACCAAGGGGCTGA
GGAGCAACGTGCTGCTGAGCGGATCCGGAAAGAACCTGCATCTGGAGCTGACC
GAGACTTGCCTGGACATGATGGCCAGATACGTGTTCTCCAATTTCACAGCTGTGC
CAAAGAGGAGCCCCGTGGGCGAGTTCTTGCTGGCCGGAGGCAGAACAAAGACCT
GGCTGGTCGGCAACAAGCTTGTGACCGTGACCACTAGCGTCGGCACCGGAACCA
GAAGCCTGCTGGGCGGCAGCGGAGGGCTCGCCGCCTATGTGCCACTGCTGACG
CAAGGATGGGCCGAGATCCTGGTCCGGAGGCCAACCGGCAACACATCCTGGCTG
ATGTCACTGGAGAATCCCGGATCTGGTATCAATCCAAGTTTCGTGTTCCTGCAG
CTGTACCACAGCCCCTTCTTCGGCGACGAGTCCAATAAGCCTATCCTGCTGCCTA
ACGAGTCCCAGAGCTTTGAGCGCAGCGTGCAACTGTTGGACCAGATCCCCAGCT
ATGACACTCACAAGATCGCCGTGCTGTATGTGGGCGAGGGACAGAGCAACTCAG
AGCTGGCCATCCTGTCCAACGAGCACGGCAGCTACAGATACACCGAGTTCCTGA
CCGGCCTGGGCCGCCTGATCGAGCTGAAAGACTGCCAGCCAGATAAGGTGTATC
TGGGCGGCCTGGACGTGTGCGGCGAGGACGGACAGTTTACATACTGCTGGCATG
ACGATATTATGCAGGCCGTGTTCCACATTGCCACACTGATGCCAACCAAAGACGT
GGACAAGCACAGGTGCGATAAGAAGAGACACCTGGGTAATGACTTCGTTTCAAT
CGTGTACAATGATAGCGGCGAGGACTTCAAACTGGGCACCATCAAGGGCCAATT
TAATTTTGTGCACGTCATCGTGACACCCCTGGACTACGAGTGCAACCTCGTGTCA
CTGCAGTGTCGGAAGGACATGGAAGGCCTGGTGGATACCTCAGTGGCTAAGATT
GTGTCCGACCGCAACCTGCCATTCGTGGCCCGACAGATGGCCCTGCATGCCAATA
TGGCCAGCCAGGTGCACCATTCACGCTCCAATCCTACTGATATCTACCCCTCTAA
GTGGATCGCCAGGCTGAGGCACATCAAGAGACTGCGGCAGCGGATCTGTGAGGA
GGCCGCCTATAGCAACCCTAGCTGAGGCCGGGGCCCTCCCTCCTGCACTGGCCTT
GGACGGTATTGCCTGTCAGTGAAATAAATAAAGTCCTGACCCCAGTGCACAGAC
ATAGAGGCACAGATTGCAGTCAGACAGCTCTTTTATTGACTTTGTCTGCTTGGTG
CGGGGGTTGGGGGGGTGTCGAGGCTCTAGAAGCGGCCATGCCCACAGAAGTGGT
ACACAGAAGCAGGCACAGCCAGCTCCGAGGGCacccagctttcttgtacaaagtgggaattcctagagc
tcgctgatcagcctcgactgtgccttctagttgccagccatctgttgtttgcccctcccccgtgccttccttgaccctggaaggtgccactc
ccactgtcctttcctaataaaatgaggaaattgcatcgcattgtctgagtaggtgtcattctattctggggggtggggtggggcaggaca
gcaagggggaggattgggaagagaatagcaggcatgctggggagggccgcaggaacccctagtgatggagttggccactccctct
ctgcgcgctcgctcgctcactgaggccgggcgaccaaaggtcgcccgacgcccgggctttgcccgggcggcctcagtgagcgag
cgagcgcgcag
SEQ ID NO: 11, Protein, Artificial sequence, Micro-Tuberin 2 protein sequence
MPLEARHAVLALLKAIVQGQGERLGVLRALFFKVIKDYPSNEDLHERLEVFKALTDN
GRHITYLEEELADFVLQWMDVGLSSEFLLVLVNLVKFNSCYLDEYIARMVQMICLLC
VRTASSVDIEVSLQVLDAVVCYNCLPAESLPLFIVTLCRTINVKELCEPCWKLMRNLL
GTHLGHSAIYNMCHLMEDRAYMEDAPLLRGAVFFVGMALWGAHRLYSLRNSPTSV
LPSFYQAMACPNEVVSYEIVLSITRLIKKYRKELQVVAWDILLNIIERLLQQLQTLDSP
ELRTIVHDLLTTVEELCDQNEFHGSQERYFELVERCADQRPESSLLNLISYRAQSIHPA
KDGWIQNLQALMERFFRSESRGAVRIKVLDVLSFVLLINRQFYEEELINSVVISQLSHI
PEDKDHQVRKLATQLLVDLAEGCHTHHFNSLLDIIEKVMARSLSPPPELEERDVAAY
SASLEDVKTAVLGLLVILQTKLGSSGSSGSSGSSMPDIIIKALPVLVVKLTHISATASM
AVPLLEFLSTLARLPHLYRNFAAEQYASVFAISLPYTNPSKFNQYIVCLAHHVIAMWF
IRCRLPFRKDFVPFITKGLRSNVLLSGSGKNLHLELTETCLDMMARYVFSNFTAVPKR
SPVGEFLLAGGRTKTWLVGNKLVTVTTSVGTGTRSLLGGSGGLAAYVPLLTQGWA
EILVRRPTGNTSWLMSLENPGSGINPSFVFLQLYHSPFFGDESNKPILLPNESQSFERS
VQLLDQIPSYDTHKIAVLYVGEGQSNSELAILSNEHGSYRYTEFLTGLGRLIELKDCQ
PDKVYLGGLDVCGEDGQFTYCWHDDIMQAVFHIATLMPTKDVDKHRCDKKRHLG
NDFVSIVYNDSGEDFKLGTIKGQFNFVHVIVTPLDYECNLVSLQCRKDMEGLVDTSV
AKIVSDRNLPFVARQMALHANMASQVHHSRSNPTDIYPSKWIARLRHIKRLRQRICE
EAAYSNPS*
SEQ ID NO: 12, DNA, Artificial sequence, Micro-Tuberin 3 coding sequence
atgccgctggaggcccggcacgcggtgctggctctgctgaaggccatcgtgcaggggcagggcgagcgtttgggggtcctcagag
ccctcttctttaaggtcatcaaggattacccttccaacgaagaccttcacgaaaggctggaggttttcaaggccctcacagacaatggga
gacacatcacctacttggaggaagagctggctgactttgtcctgcagtggatggatgttggcttgtcctcggaattccttctggtgctggt
gaacttggtcaaattcaatagctgttacctcgacgagtacatcgcaaggatggttcagatgatctgtctgctgtgcgtccggaccgcgtc
ctctgtggacatagaggtctccctgcaggtgctggacgccgtggtctgctacaactgcctgccggctgagagcctcccgctgttcatcg
ttaccctctgtcgcaccatcaacgtcaaggagctctgcgagccttgctggaagctgatgcggaacctccttggcacccacctgggcca
cagcgccatctacaacatgtgccacctcatggaggacagagcctacatggaggacgcgcccctgctgagaggagccgtgttttttgtg
ggcatggctctctggggagcccaccggctctattctctcaggaactcgccgacatctgtgttgccatcattttaccaggccatggcatgt
ccgaacgaggtggtgtcctatgagatcgtcctgtccatcaccaggctcatcaagaagtataggaaggagctccaggtggtggcgtgg
gacattctgctgaacatcatcgaacggctccttcagcagctccagaccggctcctctggtagctctggaagttccggctccagcggc
tcctccggatctgcaagccacgccacgcgtgtgtatgagatgctggtcagccacattcagctccactacaagcacagctacaccctgc
caatcgcgagcagcatccggctgcaggcctttgacttcctgttgctgctgcgggccgactcactgcaccgcctgggcctgcccaacaa
ggatggagtcgtgcggttcagcccctactgcgtctgcggcagcgggtccctgctcttccgcgtcctgctgcagtgcttgaagcaggag
tctgactggaaggtgctgaagctggttctgggcaggctgcctgagtccctgcgctataaagtgctcatctttacttccccttgcagtgtgg
accagctgtgctctgctctctgctccatgctttcaggcccaaagacactggagcggctccgaggcgccccagaaggcttctccagaac
tgacttgcacctggccgtggttccagtgctgacagcattaatctcttaccataactacctggacaaaaccaaacagcgcgagatggtcta
ctgcctggagcagggcctcatccaccgctgtgccagccagtgcgtcgtggccttgtccatctgcagcgtggagatgcctgacatcatc
atcaaggcgctgcctgttctggtggtgaagctcacgcacatctcagccacagccagcatggccgtcccactgctggagttcctgtcca
ctctggccaggctgccgcacctctacaggaactttgccgcggagcagtatgccagtgtgttcgccatctccctgccgtacaccaaccc
ctccaagtttaatcagtacatcgtgtgtctggcccatcacgtcatagccatgtggttcatcaggtgccgcctgcccttccggaaggattttg
tccctttcatcactaagggcctgcggtccaatgtcctcttgtctggctcaggaaaaaacctccacctggagctcacggaaacctgtctgg
acatgatggctcgatacgtcttctccaacttcacggctgtcccgaagaggtctcctgtgggcgagttcctcctagcgggtggcaggacc
aaaacctggctggttgggaacaagcttgtcactgtgacgacaagcgtgggaaccgggacccggtcgttactaggctccagtggcag
cctggcggcctatgtgcccctgctgacccagggctgggcggagatcctggtccggaggcccacagggaacaccagctggctgatg
agcctggagaacccgggatccggcatcaaccccagtttcgtgttcctgcagctctaccattcccccttctttggcgacgagtcaaacaa
gccaatcctgctgcccaatgagtcacagtcctttgagcggtcggtgcagctcctcgaccagatcccatcatacgacacccacaagatc
gccgtcctgtatgttggagaaggccagagcaacagcgagctcgccatcctgtccaatgagcatggctcctacaggtacacggagttc
ctgacgggcctgggccggctcatcgagctgaaggactgccagccggacaaggtgtacctgggaggcctggacgtgtgtggtgagg
acggccagttcacctactgctggcacgatgacatcatgcaagccgtcttccacatcgccaccctgatgcccaccaaggacgtggaca
agcaccgctgcgacaagaagcgccacctgggcaacgactttgtgtccattgtctacaatgactccggtgaggacttcaagcttggcac
catcaagggccagttcaactttgtccacgtgatcgtcaccccgctggactacgagtgcaacctggtgtccctgcagtgcaggaaagac
atggagggccttgtggacaccagcgtggccaagatcgtgtctgaccgcaacctgcccttcgtggcccgccagatggccctgcacgc
aaatatggcctcacaggtgcatcatagccgctccaaccccaccgatatctacccctccaagtggattgcccggctccgccacatcaag
cggctccgccagcggatctgcgaggaagccgcctactccaaccccagctga
SEQ ID NO: 13, DNA, Artificial sequence, Micro-Tuberin 3 codon optimized coding
sequence
atgcctctggaggctcggcatgccgtgctggccctgctgaaggccattgtgcaaggccagggcgaaaggttgggggtgctgagggc
cctgttctttaaggtgatcaaggactatccctccaacgaggacttgcacgagaggctggaagtgtttaaggccctgactgataacggac
gccacatcacctatctcgaggaagaactggctgactttgtgttgcagtggatggatgtcggcctgtcaagcgagttcctcctggtgctgg
tgaacctggtgaagttcaattcctgctacctggatgaatacatcgcccggatggtgcagatgatctgcctgctgtgcgtgagaaccgcct
catctgtggatatagaggtgagcctccaggtgctggacgccgtggtgtgctataattgcctgccagccgaaagcctgccactgttcatc
gtgacactgtgtaggaccattaatgtgaaggagctgtgcgagccatgctggaagctcatgaggaatctgctgggaacacacctgggc
cacagcgctatctacaacatgtgccacctgatggaggaccgggcgtacatggaggacgccccactgctgcggggcgccgtgttcttc
gtgggaatggccctgtggggggctcacagactgtactcactgcggaatagccctacctctgtgctgccttctttttatcaggctatggcct
gtcccaatgaggtggtgtcttacgagattgtgctgtctattaccagactgattaagaagtataggaaagagctgcaggtcgtggcctggg
acattctcctgaacatcatcgagcggctgctgcagcagctgcagacaggctcctctggtagctctggaagttccggctccagcggct
cctccggatctgcctctcacgccacccgggtgtacgaaatgctggtgtcccacatccagctgcattataagcacagttataccctgcct
attgccagcagcatccggctgcaggccttcgatttcctcctgctgctgcgcgccgatagcctgcaccgccttggactgccaaacaagg
atggcgtggtgagattctccccttactgcgtgtgcggcagcgggtccctgctgtttagggttctgctgcagtgtctgaagcaggagtca
gactggaaagtgctgaagctggtgctgggaagactgccagagagcctgcgatacaaggtgctgatcttcaccagcccatgctccgtg
gatcagctgtgttccgccctctgcagcatgctgagcggtcctaagaccctggaacggctgaggggcgccccagagggcttcagccg
cactgatctgcacctggctgtggtgcccgtgctgaccgccctgattagctaccataactatctggacaagaccaagcagagggagatg
gtgtactgcctggaacagggactgatccacagatgtgcttcccagtgtgtggtggccctgtccatttgctctgtggagatgcctgacatc
attatcaaagccctgccagtgctggtggtgaaactgactcacatctccgccacggcctctatggccgtccctctcctggagttcctgtcc
accctcgcccggctgcctcacctgtacaggaactttgctgccgagcagtacgcaagcgtttttgccatcagcctgccttacaccaatcct
tctaagttcaaccagtacatcgtgtgcctggcccaccatgtgattgccatgtggtttatccgctgccgcctgccctttcggaaggacttcgt
gcccttcatcaccaaggggctgaggtctaatgttctgctgtccggctcaggaaagaacctgcaccttgaactgacagaaacctgcctg
gatatgatggcccggtacgtgttttccaatttcacggctgtgcccaagcggagccctgtcggcgagttcctgctggccggaggcagga
ccaagacatggctggtgggcaacaaactggtgaccgtgaccaccagcgtgggcacaggcaccaggtcactgctgggctccagtgg
cagcctggctgcctacgtgcccctgctgacacagggctgggctgaaatcctggtgaggagaccaaccggcaacacctcttggctgat
gagcctggagaatcccggatccggcatcaatccaagcttcgtgtttctgcagctgtaccactcacccttctttggggacgagtccaaca
aacccatcctgctgcccaacgagtcccagtcttttgagagaagtgtgcagctgctggaccagattccaagttacgacactcacaagatc
gcagtgctgtatgtgggtgagggccagagcaacagcgaactggccatcctgtctaatgagcatggcagctacagatacaccgaatttc
tgaccggactgggcaggctgatcgagcttaaagactgtcagcccgacaaagtgtacctgggcggcctggacgtgtgcggggaaga
cggacagttcacctactgctggcacgacgacattatgcaggccgtgtttcacatcgccaccctgatgcctactaaagacgtggacaaa
cataggtgcgacaagaagcgccacctggggaacgactttgtgagcattgtgtacaacgactctggcgaggatttcaagctggggaca
atcaaagggcagtttaacttcgtgcacgtgatcgtgactccactggactacgagtgtaatctggtgagcctgcagtgccggaaggacat
ggagggcctggtggacacctcagtggccaagatcgtgagcgatcgtaatctgccttttgtggccagacagatggccctgcacgccaa
catggcctcccaggtgcaccactctcgctccaacccaaccgacatctacccatccaagtggatcgccagactgagacacattaagag
gctcaggcaaagaatctgtgaggaagccgcctactccaatccctcctga
SEQ ID NO: 14, DNA, Artificial sequence, Micro-Tuberin 3 Native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCGCTGGAGGCCCGGCACGCGGTGCTGGCTCTGCTGAAGGCCATCGT
GCAGGGGCAGGGCGAGCGTTTGGGGGTCCTCAGAGCCCTCTTCTTTAAGGTCATCAA
GGATTACCCTTCCAACGAAGACCTTCACGAAAGGCTGGAGGTTTTCAAGGCCCTCACA
GACAATGGGAGACACATCACCTACTTGGAGGAAGAGCTGGCTGACTTTGTCCTGCAGT
GGATGGATGTTGGCTTGTCCTCGGAATTCCTTCTGGTGCTGGTGAACTTGGTCAAATTC
AATAGCTGTTACCTCGACGAGTACATCGCAAGGATGGTTCAGATGATCTGTCTGCTGTG
CGTCCGGACCGCGTCCTCTGTGGACATAGAGGTCTCCCTGCAGGTGCTGGACGCCGT
GGTCTGCTACAACTGCCTGCCGGCTGAGAGCCTCCCGCTGTTCATCGTTACCCTCTGT
CGCACCATCAACGTCAAGGAGCTCTGCGAGCCTTGCTGGAAGCTGATGCGGAACCTC
CTTGGCACCCACCTGGGCCACAGCGCCATCTACAACATGTGCCACCTCATGGAGGAC
AGAGCCTACATGGAGGACGCGCCCCTGCTGAGAGGAGCCGTGTTTTTTGTGGGCATG
GCTCTCTGGGGAGCCCACCGGCTCTATTCTCTCAGGAACTCGCCGACATCTGTGTTGC
CATCATTTTACCAGGCCATGGCATGTCCGAACGAGGTGGTGTCCTATGAGATCGTCCT
GTCCATCACCAGGCTCATCAAGAAGTATAGGAAGGAGCTCCAGGTGGTGGCGTGGGA
CATTCTGCTGAACATCATCGAACGGCTCCTTCAGCAGCTCCAGACCGGCTCCTCTGGT
AGCTCTGGAAGTTCCGGCTCCAGCGGCTCCTCCGGATCTGCAAGCCACGCCACGCG
TGTGTATGAGATGCTGGTCAGCCACATTCAGCTCCACTACAAGCACAGCTACACCCTG
CCAATCGCGAGCAGCATCCGGCTGCAGGCCTTTGACTTCCTGTTGCTGCTGCGGGCC
GACTCACTGCACCGCCTGGGCCTGCCCAACAAGGATGGAGTCGTGCGGTTCAGCCCC
TACTGCGTCTGCGGCAGCGGGTCCCTGCTCTTCCGCGTCCTGCTGCAGTGCTTGAAG
CAGGAGTCTGACTGGAAGGTGCTGAAGCTGGTTCTGGGCAGGCTGCCTGAGTCCCTG
CGCTATAAAGTGCTCATCTTTACTTCCCCTTGCAGTGTGGACCAGCTGTGCTCTGCTCT
CTGCTCCATGCTTTCAGGCCCAAAGACACTGGAGCGGCTCCGAGGCGCCCCAGAAGG
CTTCTCCAGAACTGACTTGCACCTGGCCGTGGTTCCAGTGCTGACAGCATTAATCTCTT
ACCATAACTACCTGGACAAAACCAAACAGCGCGAGATGGTCTACTGCCTGGAGCAGGG
CCTCATCCACCGCTGTGCCAGCCAGTGCGTCGTGGCCTTGTCCATCTGCAGCGTGGA
GATGCCTGACATCATCATCAAGGCGCTGCCTGTTCTGGTGGTGAAGCTCACGCACATC
TCAGCCACAGCCAGCATGGCCGTCCCACTGCTGGAGTTCCTGTCCACTCTGGCCAGG
CTGCCGCACCTCTACAGGAACTTTGCCGCGGAGCAGTATGCCAGTGTGTTCGCCATCT
CCCTGCCGTACACCAACCCCTCCAAGTTTAATCAGTACATCGTGTGTCTGGCCCATCAC
GTCATAGCCATGTGGTTCATCAGGTGCCGCCTGCCCTTCCGGAAGGATTTTGTCCCTT
TCATCACTAAGGGCCTGCGGTCCAATGTCCTCTTGTCTGGCTCAGGAAAAAACCTCCA
CCTGGAGCTCACGGAAACCTGTCTGGACATGATGGCTCGATACGTCTTCTCCAACTTC
ACGGCTGTCCCGAAGAGGTCTCCTGTGGGCGAGTTCCTCCTAGCGGGTGGCAGGACC
AAAACCTGGCTGGTTGGGAACAAGCTTGTCACTGTGACGACAAGCGTGGGAACCGGG
ACCCGGTCGTTACTAGGCTCCAGTGGCAGCCTGGCGGCCTATGTGCCCCTGCTGACC
CAGGGCTGGGCGGAGATCCTGGTCCGGAGGCCCACAGGGAACACCAGCTGGCTGAT
GAGCCTGGAGAACCCGGGATCCGGCATCAACCCCAGTTTCGTGTTCCTGCAGCTCTA
CCATTCCCCCTTCTTTGGCGACGAGTCAAACAAGCCAATCCTGCTGCCCAATGAGTCA
CAGTCCTTTGAGCGGTCGGTGCAGCTCCTCGACCAGATCCCATCATACGACACCCACA
AGATCGCCGTCCTGTATGTTGGAGAAGGCCAGAGCAACAGCGAGCTCGCCATCCTGT
CCAATGAGCATGGCTCCTACAGGTACACGGAGTTCCTGACGGGCCTGGGCCGGCTCA
TCGAGCTGAAGGACTGCCAGCCGGACAAGGTGTACCTGGGAGGCCTGGACGTGTGTG
GTGAGGACGGCCAGTTCACCTACTGCTGGCACGATGACATCATGCAAGCCGTCTTCCA
CATCGCCACCCTGATGCCCACCAAGGACGTGGACAAGCACCGCTGCGACAAGAAGCG
CCACCTGGGCAACGACTTTGTGTCCATTGTCTACAATGACTCCGGTGAGGACTTCAAG
CTTGGCACCATCAAGGGCCAGTTCAACTTTGTCCACGTGATCGTCACCCCGCTGGACT
ACGAGTGCAACCTGGTGTCCCTGCAGTGCAGGAAAGACATGGAGGGCCTTGTGGACA
CCAGCGTGGCCAAGATCGTGTCTGACCGCAACCTGCCCTTCGTGGCCCGCCAGATGG
CCCTGCACGCAAATATGGCCTCACAGGTGCATCATAGCCGCTCCAACCCCACCGATAT
CTACCCCTCCAAGTGGATTGCCCGGCTCCGCCACATCAAGCGGCTCCGCCAGCGGAT
CTGCGAGGAAGCCGCCTACTCCAACCCCAGCTGAGGCCGGGGCCCTCCCTCCTGCA
CTGGCCTTGGACGGTATTGCCTGTCAGTGAAATAAATAAAGTCCTGACCCCAGTG
CACAGACATAGAGGCACAGATTGCAGTCAGACAGCTCTTTTATTGACTTTGTCTG
CTTGGTGCGGGGGTTGGGGGGGTGTCGAGGCTCTAGAAGCGGCCATGCCCACAG
AAGTGGTACACAGAAGCAGGCACAGCCAGCTCCGAGGGCacccagctttcttgtacaaagtggg
aattcctagagctcgctgatcagcctcgactgtgccttctagttgccagccatctgttgtttgcccctcccccgtgccttccttgaccc
tggaaggtgccactcccactgtcctttcctaataaaatgaggaaattgcatcgcattgtctgagtaggtgtcattctattctgggg
ggtggggtggggcaggacagcaagggggaggattgggaagagaatagcaggcatgctggggagggccgcaggaacccct
agtgatggagttggccactccctctctgcgcgctcgctcgctcactgaggccgggcgaccaaaggtcgcccgacgcccgggctttgc
ccgggcggcctcagtgagcgagcgagcgcgcag
SEQ ID NO: 15, DNA, Artificial sequence, Micro-Tuberin 3 codon-optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGGGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCTCTGGAGGCTCGGCATGCCGTGCTGGCCCTGCTGAAGGCCATTGTG
CAAGGCCAGGGCGAAAGGTTGGGGGTGCTGAGGGCCCTGTTCTTTAAGGTGATCAAG
GACTATCCCTCCAACGAGGACTTGCACGAGAGGCTGGAAGTGTTTAAGGCCCTGACTG
ATAACGGACGCCACATCACCTATCTCGAGGAAGAACTGGCTGACTTTGTGTTGCAGTG
GATGGATGTCGGCCTGTCAAGCGAGTTCCTCCTGGTGCTGGTGAACCTGGTGAAGTTC
AATTCCTGCTACCTGGATGAATACATCGCCCGGATGGTGCAGATGATCTGCCTGCTGT
GCGTGAGAACCGCCTCATCTGTGGATATAGAGGTGAGCCTCCAGGTGCTGGACGCCG
TGGTGTGCTATAATTGCCTGCCAGCCGAAAGCCTGCCACTGTTCATCGTGACACTGTG
TAGGACCATTAATGTGAAGGAGCTGTGCGAGCCATGCTGGAAGCTCATGAGGAATCTG
CTGGGAACACACCTGGGCCACAGCGCTATCTACAACATGTGCCACCTGATGGAGGAC
CGGGCGTACATGGAGGACGCCCCACTGCTGCGGGGCGCCGTGTTCTTCGTGGGAAT
GGCCCTGTGGGGGGCTCACAGACTGTACTCACTGCGGAATAGCCCTACCTCTGTGCT
GCCTTCTTTTTATCAGGCTATGGCCTGTCCCAATGAGGTGGTGTCTTACGAGATTGTGC
TGTCTATTACCAGACTGATTAAGAAGTATAGGAAAGAGCTGCAGGTCGTGGCCTGGGA
CATTCTCCTGAACATCATCGAGCGGCTGCTGCAGCAGCTGCAGACAGGCTCCTCTGG
TAGCTCTGGAAGTTCCGGCTCCAGCGGCTCCTCCGGATCTGCCTCTCACGCCACCC
GGGTGTACGAAATGCTGGTGTCCCACATCCAGCTGCATTATAAGCACAGTTATACCCT
GCCTATTGCCAGCAGCATCCGGCTGCAGGCCTTCGATTTCCTCCTGCTGCTGCGCGC
CGATAGCCTGCACCGCCTTGGACTGCCAAACAAGGATGGCGTGGTGAGATTCTCCCCT
TACTGCGTGTGCGGCAGCGGGTCCCTGCTGTTTAGGGTTCTGCTGCAGTGTCTGAAG
CAGGAGTCAGACTGGAAAGTGCTGAAGCTGGTGCTGGGAAGACTGCCAGAGAGCCTG
CGATACAAGGTGCTGATCTTCACCAGCCCATGCTCCGTGGATCAGCTGTGTTCCGCCC
TCTGCAGCATGCTGAGCGGTCCTAAGACCCTGGAACGGCTGAGGGGCGCCCCAGAG
GGCTTCAGCCGCACTGATCTGCACCTGGCTGTGGTGCCCGTGCTGACCGCCCTGATT
AGCTACCATAACTATCTGGACAAGACCAAGCAGAGGGAGATGGTGTACTGCCTGGAAC
AGGGACTGATCCACAGATGTGCTTCCCAGTGTGTGGTGGCCCTGTCCATTTGCTCTGT
GGAGATGCCTGACATCATTATCAAAGCCCTGCCAGTGCTGGTGGTGAAACTGACTCAC
ATCTCCGCCACGGCCTCTATGGCCGTCCCTCTCCTGGAGTTCCTGTCCACCCTCGCCC
GGCTGCCTCACCTGTACAGGAACTTTGCTGCCGAGCAGTACGCAAGCGTTTTTGCCAT
CAGCCTGCCTTACACCAATCCTTCTAAGTTCAACCAGTACATCGTGTGCCTGGCCCACC
ATGTGATTGCCATGTGGTTTATCCGCTGCCGCCTGCCCTTTCGGAAGGACTTCGTGCC
CTTCATCACCAAGGGGCTGAGGTCTAATGTTCTGCTGTCCGGCTCAGGAAAGAACCTG
CACCTTGAACTGACAGAAACCTGCCTGGATATGATGGCCCGGTACGTGTTTTCCAATTT
CACGGCTGTGCCCAAGCGGAGCCCTGTCGGCGAGTTCCTGCTGGCCGGAGGCAGGA
CCAAGACATGGCTGGTGGGCAACAAACTGGTGACCGTGACCACCAGCGTGGGCACAG
GCACCAGGTCACTGCTGGGCTCCAGTGGCAGCCTGGCTGCCTACGTGCCCCTGCTG
ACACAGGGCTGGGCTGAAATCCTGGTGAGGAGACCAACCGGCAACACCTCTTGGCTG
ATGAGCCTGGAGAATCCCGGATCCGGCATCAATCCAAGCTTCGTGTTTCTGCAGCTGT
ACCACTCACCCTTCTTTGGGGACGAGTCCAACAAACCCATCCTGCTGCCCAACGAGTC
CCAGTCTTTTGAGAGAAGTGTGCAGCTGCTGGACCAGATTCCAAGTTACGACACTCAC
AAGATCGCAGTGCTGTATGTGGGTGAGGGCCAGAGCAACAGCGAACTGGCCATCCTG
TCTAATGAGCATGGCAGCTACAGATACACCGAATTTCTGACCGGACTGGGCAGGCTGA
TCGAGCTTAAAGACTGTCAGCCCGACAAAGTGTACCTGGGCGGCCTGGACGTGTGCG
GGGAAGACGGACAGTTCACCTACTGCTGGCACGACGACATTATGCAGGCCGTGTTTCA
CATCGCCACCCTGATGCCTACTAAAGACGTGGACAAACATAGGTGCGACAAGAAGCGC
CACCTGGGGAACGACTTTGTGAGCATTGTGTACAACGACTCTGGCGAGGATTTCAAGC
TGGGGACAATCAAAGGGCAGTTTAACTTCGTGCACGTGATCGTGACTCCACTGGACTA
CGAGTGTAATCTGGTGAGCCTGCAGTGCCGGAAGGACATGGAGGGCCTGGTGGACAC
CTCAGTGGCCAAGATCGTGAGCGATCGTAATCTGCCTTTTGTGGCCAGACAGATGGCC
CTGCACGCCAACATGGCCTCCCAGGTGCACCACTCTCGCTCCAACCCAACCGACATCT
ACCCATCCAAGTGGATCGCCAGACTGAGACACATTAAGAGGCTCAGGCAAAGAATCTG
TGAGGAAGCCGCCTACTCCAATCCCTCCTGAGGCCGGGGCCCTCCCTCCTGCACTG
GCCTTGGACGGTATTGCCTGTCAGTGAAATAAATAAAGTCCTGACCCCAGTGCAC
AGACATAGAGGCACAGATTGCAGTCAGACAGCTCTTTTATTGACTTTGTCTGCTT
GGTGCGGGGGTTGGGGGGGTGTCGAGGCTCTAGAAGCGGCCATGCCCACAGAAG
TGGTACACAGAAGCAGGCACAGCCAGCTCCGAGGGCacccagctttcttgtacaaagtgggaattc
ctagagctcgctgatcagcctcgactgtgccttctagttgccagccatctgttgtttgcccctcccccgtgccttccttgaccctgga
aggtgccactcccactgtcctttcctaataaaatgaggaaattgcatcgcattgtctgagtaggtgtcattctattctggggggtg
gggtggggcaggacagcaagggggaggattgggaagagaatagcaggcatgctggggagggccgcaggaacccctagtga
tggagttggccactccctctctgcgcgctcgctcgctcactgaggccgggcgaccaaaggtcgcccgacgcccgggctttgcccgg
gcggcctcagtgagcgagcgagcgcgcag
SEQ ID NO: 16, Protein, Artificial sequence, Micro-Tuberin 3 protein sequence
MPLEARHAVLALLKAIVQGQGERLGVLRALFFKVIKDYPSNEDLHERLEVFKALTDN
GRHITYLEEELADFVLQWMDVGLSSEFLLVLVNLVKFNSCYLDEYIARMVQMICLLC
VRTASSVDIEVSLQVLDAVVCYNCLPAESLPLFIVTLCRTINVKELCEPCWKLMRNLL
GTHLGHSAIYNMCHLMEDRAYMEDAPLLRGAVFFVGMALWGAHRLYSLRNSPTSV
LPSFYQAMACPNEVVSYEIVLSITRLIKKYRKELQVVAWDILLNIIERLLQQLQTGSSG
SSGSSGSSGSSGSASHATRVYEMLVSHIQLHYKHSYTLPIASSIRLQAFDFLLLLRADS
LHRLGLPNKDGVVRFSPYCVCGSGSLLFRVLLQCLKQESDWKVLKLVLGRLPESLR
YKVLIFTSPCSVDQLCSALCSMLSGPKTLERLRGAPEGFSRTDLHLAVVPVLTALISY
HNYLDKTKQREMVYCLEQGLIHRCASQCVVALSICSVEMPDIIIKALPVLVVKLTHIS
ATASMAVPLLEFLSTLARLPHLYRNFAAEQYASVFAISLPYTNPSKFNQYIVCLAHHV
IAMWFIRCRLPFRKDFVPFITKGLRSNVLLSGSGKNLHLELTETCLDMMARYVFSNF
TAVPKRSPVGEFLLAGGRTKTWLVGNKLVTVTTSVGTGTRSLLGSSGSLAAYVPLLT
QGWAEILVRRPTGNTSWLMSLENPGSGINPSFVFLQLYHSPFFGDESNKPILLPNESQ
SFERSVQLLDQIPSYDTHKIAVLYVGEGQSNSELAILSNEHGSYRYTEFLTGLGRLIEL
KDCQPDKVYLGGLDVCGEDGQFTYCWHDDIMQAVFHIATLMPTKDVDKHRCDKK
RHLGNDFVSIVYNDSGEDFKLGTIKGQFNFVHVIVTPLDYECNLVSLQCRKDMEGLV
DTSVAKIVSDRNLPFVARQMALHANMASQVHHSRSNPTDIYPSKWIARLRHIKRLRQ
RICEEAAYSNPS*
SEQ ID NO: 17, DNA, Artificial sequence, Micro-Tuberin 5 coding sequence
atgccgctggaggcccggcacgcggtgctggctctgctgaaggccatcgtgcaggggcagggcgagcgtttgggggtcctcagag
ccctcttctttaaggtcatcaaggattacccttccaacgaagaccttcacgaaaggctggaggttttcaaggccctcacagacaatggga
gacacatcacctacttggaggaagagctggctgactttgtcctgcagtggatggatgttggcttgtcctcggaattccttctggtgctggt
gaacttggtcaaattcaatagcggatctggatgcctgccggctgagagcctcccgctgttcatgttaccctctgtcgcaccatcaacgt
caaggagctctgcgagccttgctggaagctgatgcggaacctccttggcacccacggctcctccggctctagcggcagcgtggtgg
cgtgggacattctgctgaacatcatcgaacggctccttcagcagctccagaccttggacagcccggagctcaggaccatcgtccatga
cctgttgaccacggtggaggagctgtgtgaccagaacgagttccacgggtctcaggagagatactttgaactggtggagagatgtgc
ggaccagaggcctggcagctccggaggcgccgtgcgcatcaaggtgctggacgtgctgtcctttgtgctgctcatcaacaggcagtt
ctatgaggaggagctgattaactcagtggtcatctcgcagctctcccacatccccgaggataaagaccaccaggtccgaaagctggc
cacccagttgctggtggacctggcagagggctgccacgggagtggcgcaagccacgccacgcgtgtgtatgagatgctggtcagc
cacattcagctccactacaagcacagctacaccctgccaatcgcgagcagcatccggctgcaggcctttgacttcctgttgctgctgcg
ggccgactcactgcaccgcctgggcctgcccaacaaggatggagtcgtgcggttcagcccctactgcgtctgcggcagtgggtccc
tgctcttccgcgtcctgctgcagtgcttgaagcaggagtctgactggaaggtgctgaagctggttctgggcaggctgcctgagtccctg
cgctataaagtgctcatctttacttccccttgcagtgtggaccagctgtgctctgctctctgctccatgctttcaggcccaaagacactgga
gcggctccgaggcgccccagaaggcttctccagaactgacttgcacctggccgtggttccagtgctgacagcattaatctcttaccata
actacctggacaaaaccaaacagcgcgagatggtctactgcctggagcagggcctcatccaccgctgtgccagccagtgcgtcgtg
gccttgtccatctgcagcgtggagatgcctgacatcatcatcaaggcgctgcctgttctggtggtgaagctcacgcacatctcagccac
agccagcatggccgtcccactgctggagttcctgtccactctggccaggctgccgcacctctacaggaactttgccgcggagcagtat
gccagtgtgttcgccatctccctgccgtacaccaacccctccaagtttaatcagtacatcgtgtgtctggcccatcacgtcatagccatgt
ggttcatcaggtgccgcctgcccttccggaaggattttgtccctttcatcactaagggcctgcggtccaatgtcctcttgtctggctccgg
taaaaacctccacctggagctcacggaaacctgtctggacatgatggctcgatacgtcttctccaacttcacggctgtcccgaagaggt
ctcctgtgggcgagttcctcctagcgggtggcaggaccaaaacctggctggttgggaacaagcttgtcactgtgacgacaagcgtgg
gaaccgggacccggtcgttactaggaagctctggctctctggcggcctatgtgcccctgctgacccagggctgggcggagatcctg
gtccggaggcccacagggaacaccagctggctgatgagcctggagaacccgggctccggcatcaaccccagtttcgtgttcctgca
gctctaccattcccccttctttggcgacgagtcaaacaagccaatcctgctgcccaatgagtcacagtcctttgagcggtcggtgcagct
cctcgaccagatcccatcatacgacacccacaagatcgccgtcctgtatgttggagaaggccagagcaacagcgagctcgccatcct
gtccaatgagcatggctcctacaggtacacggagttcctgacgggcctgggccggctcatcgagctgaaggactgccagccggaca
aggtgtacctgggaggcctggacgtgtgtggtgaggacggccagttcacctactgctggcacgatgacatcatgcaagccgtcttcca
catcgccaccctgatgcccaccaaggacgtggacaagcaccgctgcgacaagaagcgccacctgggcaacgactttgtgtccattgt
ctacaatgactccggtgaggacttcaagcttggcaccatcaagggccagttcaactttgtccacgtgatcgtcaccccgctggactacg
agtgcaacctggtgtccctgcagtgcaggaaagacatggagggccttgtggacaccagcgtggccaagatcgtgtctgaccgcaac
ctgcccttcgtggcccgccagatggccctgcacgcaaatatggcctcacaggtgcatcatagccgctccaaccccaccgatatctacc
cctccaagtggattgcccggctccgccacatcaagcggctccgccagcggatctgcgaggaagccgcctactccaaccccagctga
SEQ ID NO: 18, DNA, Artificial sequence, Micro-Tuberin 5 codon-optimized coding
sequence
atgcctctggaggctaggcacgccgtgctggccctgctgaaggccatcgtgcagggccagggcgagcgcttgggcgtgctgagag
ccctgttttttaaggtgattaaggactatccctccaacgaggatctgcacgagagactggaggtgttcaaagctctgacagacaacgga
agacacatcacctatctggaggaggagctggccgactttgtgctgcagtggatggatgtgggactgagctctgagtttctgctggtgct
ggtgaatctggtgaagttcaacagcggatctggatgtctccctgccgagagcctgccactgtttatcgtgaccctgtgcaggaccatca
acgtgaaggaactgtgtgagccctgttggaagctgatgagaaacctgctgggtacccacggctcctccggctctagcggcagcgtg
gtggcttgggacatcctgttgaacatcattgaaagactgctgcagcagctccagactctggacagtcccgaacttaggacaatcgtgca
cgacctactgaccaccgtggaggagctgtgcgatcagaacgagttccacggaagccaggagaggtatttcgaactggtggaaagat
gcgctgaccagaggcctggcagctccggaggcgccgtgagaatcaaggtgctggatgtgctgagctttgtgctgctgattaaccggc
agttctacgaggaggaactgattaatagcgttgtgatttcacagctctcacacattcccgaggataaggatcaccaggtcaggaagctg
gccacccagctcctggtggacctggccgagggttgccacgggagtggcgcctcacacgccacccgggtgtacgaaatgctcgtga
gtcacatccagctgcactacaagcacagctatacactgcccatcgcttccagcattcgcctgcaggcctttgactttctgctgctgctgag
agccgatagtctgcaccggctgggcctgcccaacaaggacggggtggtgaggttcagtccatattgcgtgtgcggcagtgggtcact
gctgttcagggtgctgctgcagtgcctgaaacaggagtctgattggaaagtgctgaagctggtgctgggcagacttcccgagagcctg
agatacaaggtgctgatcttcacctctccatgcagtgtggaccaactgtgtagcgccctgtgctccatgctgagcggcccaaagaccct
ggaacggctgagaggcgcccccgaaggatttagcaggaccgacctgcacctggctgtggtgcccgtcctgacagccctgattagct
accacaactacctggacaagacaaagcagagggagatggtgtactgcctggagcagggactgatccaccgctgtgccagccagtg
cgtggtggccctgagcatctgttccgtggagatgcctgatattatcatcaaggccctgcccgtgctggtggtgaagctgacacacatta
gcgcaactgcttccatggccgtgcctctgctggagttcctgagtacactggcccggctgccccacctctatagaaactttgccgccgag
cagtacgcctccgtgttcgctatctctctgccatacacaaacccctctaagttcaaccagtacatcgtgtgcctggcacaccacgttatcg
ccatgtggttcatccgctgtaggctgcccttccgcaaggattttgtgccgtttatcaccaagggcctgcggtccaacgtgctgctgagtg
gctccggtaagaacctccatctggagctgaccgagacctgtctggacatgatggcaaggtacgtgttcagcaacttcacagccgtgcc
aaagcggtcccccgtgggcgagtttctgcttgctggcgggcggacaaagacctggctcgtgggaaataagctggtgaccgttaccac
ctccgtgggaactggcaccagatctctgctcggaagctctggctctctggccgcctatgtgcccctgctgacccagggatgggccga
aatcctggtgcgaaggccaacaggcaacacctcctggctgatgagcctggagaatcctggctccggcatcaacccttctttcgtgttcc
tgcagctgtaccattcccccttcttcggcgacgagagcaacaagccaatcctgctgcctaacgagtcccagagcttcgagcgatcggt
gcagctgctggaccagatccccagttatgacacccataaaatcgccgtgctgtacgtgggagagggccagagcaacagcgagctgg
ccatcctgtcaaatgaacacggcagctacaggtatactgagttcctcacaggcctgggaagactgatcgagctgaaggattgccagcc
tgataaggtgtacctgggaggcctggacgtgtgcggtgaagacgggcagtttacctactgctggcatgatgatatcatgcaggcagtg
ttccatatcgccacactgatgcccaccaaggatgtggacaaacaccgctgcgataagaaaaggcacctgggcaacgacttcgtgagt
atcgtgtacaacgacagcggggaggacttcaaactgggcaccattaaaggacagttcaattttgtccacgtgatcgtgactcccctgga
ctacgagtgcaacctggtgagtctgcagtgccgcaaagatatggagggcctggtggacacctccgtggctaagatcgtgagcgaca
gaaacctgcctttcgtggcacggcagatggccctgcacgccaacatggccagccaggtgcaccactcaaggtccaaccccaccgac
atctatcccagcaaatggatcgctcgcctgcggcacatcaaacggctcaggcagcggatctgtgaggaggctgcctatagcaacccc
agttga
SEQ ID NO: 19, DNA, Artificial sequence, Micro-Tuberin 5 native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCGCTGGAGGCCCGGCACGCGGTGCTGGCTCTGCTGAAGGCCATC
GTGCAGGGGCAGGGCGAGCGTTTGGGGGTCCTCAGAGCCCTCTTCTTTAAGGTCA
TCAAGGATTACCCTTCCAACGAAGACCTTCACGAAAGGCTGGAGGTTTTCAAGG
CCCTCACAGACAATGGGAGACACATCACCTACTTGGAGGAAGAGCTGGCTGACT
TTGTCCTGCAGTGGATGGATGTTGGCTTGTCCTCGGAATTCCTTCTGGTGCTGGTG
AACTTGGTCAAATTCAATAGCGGATCTGGATGCCTGCCGGCTGAGAGCCTCCCG
CTGTTCATCGTTACCCTCTGTCGCACCATCAACGTCAAGGAGCTCTGCGAGCCTT
GCTGGAAGCTGATGCGGAACCTCCTTGGCACCCACGGCTCCTCCGGCTCTAGC
GGCAGCGTGGTGGCGTGGGACATTCTGCTGAACATCATCGAACGGCTCCTTCAG
CAGCTCCAGACCTTGGACAGCCCGGAGCTCAGGACCATCGTCCATGACCTGTTGA
CCACGGTGGAGGAGCTGTGTGACCAGAACGAGTTCCACGGGTCTCAGGAGAGAT
ACTTTGAACTGGTGGAGAGATGTGCGGACCAGAGGCCTGGCAGCTCCGGAGGC
GCCGTGCGCATCAAGGTGCTGGACGTGCTGTCCTTTGTGCTGCTCATCAACAGGC
AGTTCTATGAGGAGGAGCTGATTAACTCAGTGGTCATCTCGCAGCTCTCCCACAT
CCCCGAGGATAAAGACCACCAGGTCCGAAAGCTGGCCACCCAGTTGCTGGTGGA
CCTGGCAGAGGGCTGCCACGGGAGTGGCGCAAGCCACGCCACGCGTGTGTATG
AGATGCTGGTCAGCCACATTCAGCTCCACTACAAGCACAGCTACACCCTGCCAAT
CGCGAGCAGCATCCGGCTGCAGGCCTTTGACTTCCTGTTGCTGCTGCGGGCCGAC
TCACTGCACCGCCTGGGCCTGCCCAACAAGGATGGAGTCGTGCGGTTCAGCCCCT
ACTGCGTCTGCGGCAGTGGGTCCCTGCTCTTCCGCGTCCTGCTGCAGTGCTTGAA
GCAGGAGTCTGACTGGAAGGTGCTGAAGCTGGTTCTGGGCAGGCTGCCTGAGTC
CCTGCGCTATAAAGTGCTCATCTTTACTTCCCCTTGCAGTGTGGACCAGCTGTGCT
CTGCTCTCTGCTCCATGCTTTCAGGCCCAAAGACACTGGAGCGGCTCCGAGGCGC
CCCAGAAGGCTTCTCCAGAACTGACTTGCACCTGGCCGTGGTTCCAGTGCTGACA
GCATTAATCTCTTACCATAACTACCTGGACAAAACCAAACAGCGCGAGATGGTCT
ACTGCCTGGAGCAGGGCCTCATCCACCGCTGTGCCAGCCAGTGCGTCGTGGCCTT
GTCCATCTGCAGCGTGGAGATGCCTGACATCATCATCAAGGCGCTGCCTGTTCTG
GTGGTGAAGCTCACGCACATCTCAGCCACAGCCAGCATGGCCGTCCCACTGCTG
GAGTTCCTGTCCACTCTGGCCAGGCTGCCGCACCTCTACAGGAACTTTGCCGCGG
AGCAGTATGCCAGTGTGTTCGCCATCTCCCTGCCGTACACCAACCCCTCCAAGTT
TAATCAGTACATCGTGTGTCTGGCCCATCACGTCATAGCCATGTGGTTCATCAGG
TGCCGCCTGCCCTTCCGGAAGGATTTTGTCCCTTTCATCACTAAGGGCCTGCGGT
CCAATGTCCTCTTGTCTGGCTCCGGTAAAAACCTCCACCTGGAGCTCACGGAAA
CCTGTCTGGACATGATGGCTCGATACGTCTTCTCCAACTTCACGGCTGTCCCGAA
GAGGTCTCCTGTGGGCGAGTTCCTCCTAGCGGGTGGCAGGACCAAAACCTGGCT
GGTTGGGAACAAGCTTGTCACTGTGACGACAAGCGTGGGAACCGGGACCCGGTC
GTTACTAGGAAGCTCTGGCTCTCTGGCGGCCTATGTGCCCCTGCTGACCCAGGG
CTGGGCGGAGATCCTGGTCCGGAGGCCCACAGGGAACACCAGCTGGCTGATGAG
CCTGGAGAACCCGGGCTCCGGCATCAACCCCAGTTTCGTGTTCCTGCAGCTCTA
CCATTCCCCCTTCTTTGGCGACGAGTCAAACAAGCCAATCCTGCTGCCCAATGAG
TCACAGTCCTTTGAGCGGTCGGTGCAGCTCCTCGACCAGATCCCATCATACGACA
CCCACAAGATCGCCGTCCTGTATGTTGGAGAAGGCCAGAGCAACAGCGAGCTCG
CCATCCTGTCCAATGAGCATGGCTCCTACAGGTACACGGAGTTCCTGACGGGCCT
GGGCCGGCTCATCGAGCTGAAGGACTGCCAGCCGGACAAGGTGTACCTGGGAGG
CCTGGACGTGTGTGGTGAGGACGGCCAGTTCACCTACTGCTGGCACGATGACATC
ATGCAAGCCGTCTTCCACATCGCCACCCTGATGCCCACCAAGGACGTGGACAAG
CACCGCTGCGACAAGAAGCGCCACCTGGGCAACGACTTTGTGTCCATTGTCTACA
ATGACTCCGGTGAGGACTTCAAGCTTGGCACCATCAAGGGCCAGTTCAACTTTGT
CCACGTGATCGTCACCCCGCTGGACTACGAGTGCAACCTGGTGTCCCTGCAGTGC
AGGAAAGACATGGAGGGCCTTGTGGACACCAGCGTGGCCAAGATCGTGTCTGAC
CGCAACCTGCCCTTCGTGGCCCGCCAGATGGCCCTGCACGCAAATATGGCCTCAC
AGGTGCATCATAGCCGCTCCAACCCCACCGATATCTACCCCTCCAAGTGGATTGC
CCGGCTCCGCCACATCAAGCGGCTCCGCCAGCGGATCTGCGAGGAAGCCGCCTA
CTCCAACCCCAGCTGAGGCCGGGGCCCTCCCTCCTGCACTGGCCTTGGACGGTAT
TGCCTGTCAGTGAAATAAATAAAGTCCTGACCCCAGTGCACAGACATAGAGGCA
CAGATTGCAGTCAGACAGCTCTTTTATTGACTTTGTCTGCTTGGTGCGGGGGTTG
GGGGGGTGTCGAGGCTCTAGAAGCGGCCATGCCCACAGAAGTGGTACACAGAAG
CAGGCACAGCCAGCTCCGAGGGCacccagctttcttgtacaaagtgggaattcctagagctcgctgatcagcctc
gactgtgccttctagttgccagccatctgttgtttgcccctcccccgtgccttccttgaccctggaaggtgccactcccactgtcctt
tcctaataaaatgaggaaattgcatcgcattgtctgagtaggtgtcattctattctggggggtggggtggggcaggacagcaa
gggggaggattgggaagagaatagcaggcatgctggggagggccgcaggaacccctagtgatggagttggccactccctctct
gcgcgctcgctcgctcactgaggccgggcgaccaaaggtcgcccgacgcccgggctttgcccgggcggcctcagtgagcgagcg
agcgcgcag
SEQ ID NO: 20, DNA, Artificial sequence, Micro-Tuberin 5 codon optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGCCTCTGGAGGCTAGGCACGCCGTGCTGGCCCTGCTGAAGGCCATC
GTGCAGGGCCAGGGCGAGCGCTTGGGCGTGCTGAGAGCCCTGTTTTTTAAGGTG
ATTAAGGACTATCCCTCCAACGAGGATCTGCACGAGAGACTGGAGGTGTTCAAA
GCTCTGACAGACAACGGAAGACACATCACCTATCTGGAGGAGGAGCTGGCCGAC
TTTGTGCTGCAGTGGATGGATGTGGGACTGAGCTCTGAGTTTCTGCTGGTGCTGG
TGAATCTGGTGAAGTTCAACAGCGGATCTGGATGTCTCCCTGCCGAGAGCCTGC
CACTGTTTATCGTGACCCTGTGCAGGACCATCAACGTGAAGGAACTGTGTGAGCC
CTGTTGGAAGCTGATGAGAAACCTGCTGGGTACCCACGGCTCCTCCGGCTCTA
GCGGCAGCGTGGTGGCTTGGGACATCCTGTTGAACATCATTGAAAGACTGCTGC
AGCAGCTCCAGACTCTGGACAGTCCCGAACTTAGGACAATCGTGCACGACCTAC
TGACCACCGTGGAGGAGCTGTGCGATCAGAACGAGTTCCACGGAAGCCAGGAGA
GGTATTTCGAACTGGTGGAAAGATGCGCTGACCAGAGGCCTGGCAGCTCCGGA
GGCGCCGTGAGAATCAAGGTGCTGGATGTGCTGAGCTTTGTGCTGCTGATTAACC
GGCAGTTCTACGAGGAGGAACTGATTAATAGCGTTGTGATTTCACAGCTCTCACA
CATTCCCGAGGATAAGGATCACCAGGTCAGGAAGCTGGCCACCCAGCTCCTGGT
GGACCTGGCCGAGGGTTGCCACGGGAGTGGCGCCTCACACGCCACCCGGGTGT
ACGAAATGCTCGTGAGTCACATCCAGCTGCACTACAAGCACAGCTATACACTGC
CCATCGCTTCCAGCATTCGCCTGCAGGCCTTTGACTTTCTGCTGCTGCTGAGAGCC
GATAGTCTGCACCGGCTGGGCCTGCCCAACAAGGACGGGGTGGTGAGGTTCAGT
CCATATTGCGTGTGCGGCAGTGGGTCACTGCTGTTCAGGGTGCTGCTGCAGTGC
CTGAAACAGGAGTCTGATTGGAAAGTGCTGAAGCTGGTGCTGGGCAGACTTCCC
GAGAGCCTGAGATACAAGGTGCTGATCTTCACCTCTCCATGCAGTGTGGACCAAC
TGTGTAGCGCCCTGTGCTCCATGCTGAGCGGCCCAAAGACCCTGGAACGGCTGA
GAGGCGCCCCCGAAGGATTTAGCAGGACCGACCTGCACCTGGCTGTGGTGCCCG
TCCTGACAGCCCTGATTAGCTACCACAACTACCTGGACAAGACAAAGCAGAGGG
AGATGGTGTACTGCCTGGAGCAGGGACTGATCCACCGCTGTGCCAGCCAGTGCG
TGGTGGCCCTGAGCATCTGTTCCGTGGAGATGCCTGATATTATCATCAAGGCCCT
GCCCGTGCTGGTGGTGAAGCTGACACACATTAGCGCAACTGCTTCCATGGCCGTG
CCTCTGCTGGAGTTCCTGAGTACACTGGCCCGGCTGCCCCACCTCTATAGAAACT
TTGCCGCCGAGCAGTACGCCTCCGTGTTCGCTATCTCTCTGCCATACACAAACCC
CTCTAAGTTCAACCAGTACATCGTGTGCCTGGCACACCACGTTATCGCCATGTGG
TTCATCCGCTGTAGGCTGCCCTTCCGCAAGGATTTTGTGCCGTTTATCACCAAGG
GCCTGCGGTCCAACGTGCTGCTGAGTGGCTCCGGTAAGAACCTCCATCTGGAGC
TGACCGAGACCTGTCTGGACATGATGGCAAGGTACGTGTTCAGCAACTTCACAG
CCGTGCCAAAGCGGTCCCCCGTGGGCGAGTTTCTGCTTGCTGGCGGGCGGACAA
AGACCTGGCTCGTGGGAAATAAGCTGGTGACCGTTACCACCTCCGTGGGAACTG
GCACCAGATCTCTGCTCGGAAGCTCTGGCTCTCTGGCCGCCTATGTGCCCCTGC
TGACCCAGGGATGGGCCGAAATCCTGGTGCGAAGGCCAACAGGCAACACCTCCT
GGCTGATGAGCCTGGAGAATCCTGGCTCCGGCATCAACCCTTCTTTCGTGTTCCT
GCAGCTGTACCATTCCCCCTTCTTCGGCGACGAGAGCAACAAGCCAATCCTGCTG
CCTAACGAGTCCCAGAGCTTCGAGCGATCGGTGCAGCTGCTGGACCAGATCCCC
AGTTATGACACCCATAAAATCGCCGTGCTGTACGTGGGAGAGGGCCAGAGCAAC
AGCGAGCTGGCCATCCTGTCAAATGAACACGGCAGCTACAGGTATACTGAGTTC
CTCACAGGCCTGGGAAGACTGATCGAGCTGAAGGATTGCCAGCCTGATAAGGTG
TACCTGGGAGGCCTGGACGTGTGCGGTGAAGACGGGCAGTTTACCTACTGCTGG
CATGATGATATCATGCAGGCAGTGTTCCATATCGCCACACTGATGCCCACCAAGG
ATGTGGACAAACACCGCTGCGATAAGAAAAGGCACCTGGGCAACGACTTCGTGA
GTATCGTGTACAACGACAGCGGGGAGGACTTCAAACTGGGCACCATTAAAGGAC
AGTTCAATTTTGTCCACGTGATCGTGACTCCCCTGGACTACGAGTGCAACCTGGT
GAGTCTGCAGTGCCGCAAAGATATGGAGGGCCTGGTGGACACCTCCGTGGCTAA
GATCGTGAGCGACAGAAACCTGCCTTTCGTGGCACGGCAGATGGCCCTGCACGC
CAACATGGCCAGCCAGGTGCACCACTCAAGGTCCAACCCCACCGACATCTATCC
CAGCAAATGGATCGCTCGCCTGCGGCACATCAAACGGCTCAGGCAGCGGATCTG
TGAGGAGGCTGCCTATAGCAACCCCAGTTGAGGCCGGGGCCCTCCCTCCTGCACT
GGCCTTGGACGGTATTGCCTGTCAGTGAAATAAATAAAGTCCTGACCCCAGTGCA
CAGACATAGAGGCACAGATTGCAGTCAGACAGCTCTTTTATTGACTTTGTCTGCT
TGGTGCGGGGGTTGGGGGGGTGTCGAGGCTCTAGAAGCGGCCATGCCCACAGAA
GTGGTACACAGAAGCAGGCACAGCCAGCTCCGAGGGCacccagctttcttgtacaaagtgggaatt
cctagagctcgctgatcagcctcgactgtgccttctagttgccagccatctgttgtttgcccctcccccgtgccttccttgaccctgg
aaggtgccactcccactgtcctttcctaataaaatgaggaaattgcatcgcattgtctgagtaggtgtcattctattctggggggt
ggggtggggcaggacagcaagggggaggattgggaagagaatagcaggcatgctggggagggccgcaggaacccctagtg
atggagttggccactccctctctgcgcgctcgctcgctcactgaggccgggcgaccaaaggtcgcccgacgcccgggctttgcccg
ggcggcctcagtgagcgagcgagcgcgcag
SEQ ID NO: 21, DNA, Artificial sequence, Micro-Tuberin 5 protein sequence
MPLEARHAVLALLKAIVQGQGERLGVLRALFFKVIKDYPSNEDLHERLEVFKALTDN
GRHITYLEEELADFVLQWMDVGLSSEFLLVLVNLVKFNSGSGCLPAESLPLFIVTLCR
TINVKELCEPCWKLMRNLLGTHGSSGSSGSVVAWDILLNIIERLLQQLQTLDSPELRT
IVHDLLTTVEELCDQNEFHGSQERYFELVERCADQRPGSSGGAVRIKVLDVLSFVLLI
NRQFYEEELINSVVISQLSHIPEDKDHQVRKLATQLLVDLAEGCHGSGASHATRVYE
MLVSHIQLHYKHSYTLPIASSIRLQAFDFLLLLRADSLHRLGLPNKDGVVRFSPYCVC
GSGSLLFRVLLQCLKQESDWKVLKLVLGRLPESLRYKVLIFTSPCSVDQLCSALCSM
LSGPKTLERLRGAPEGFSRTDLHLAVVPVLTALISYHNYLDKTKQREMVYCLEQGLI
HRCASQCVVALSICSVEMPDIIIKALPVLVVKLTHISATASMAVPLLEFLSTLARLPHL
YRNFAAEQYASVFAISLPYTNPSKFNQYIVCLAHHVIAMWFIRCRLPFRKDFVPFITK
GLRSNVLLSGSGKNLHLELTETCLDMMARYVFSNFTAVPKRSPVGEFLLAGGRTKT
WLVGNKLVTVTTSVGTGTRSLLGSSGSLAAYVPLLTQGWAEILVRRPTGNTSWLMS
LENPGSGINPSFVFLQLYHSPFFGDESNKPILLPNESQSFERSVQLLDQIPSYDTHKIAV
LYVGEGQSNSELAILSNEHGSYRYTEFLTGLGRLIELKDCQPDKVYLGGLDVCGEDG
QFTYCWHDDIMQAVFHIATLMPTKDVDKHRCDKKRHLGNDFVSIVYNDSGEDFKL
GTIKGQFNFVHVIVTPLDYECNLVSLQCRKDMEGLVDTSVAKIVSDRNLPFVARQM
ALHANMASQVHHSRSNPTDIYPSKWIARLRHIKRLRQRICEEAAYSNPS
SEQ ID NO: 22, DNA, Artificial sequence, Micro-Tuberin 6 coding sequence
atggaatgtggcctcaacaatcgcatccggatgatagggcagatttgtgaagtcgcaaaaaccaagaaatttgaagagcacgcagtg
gaagcactctggaaggcggtcgcggatctgttgcagccggagcggccgctggaggcccggcacgcggtgctggctctgctgaagg
ccatcgtgcaggggcagggcgagcgtttgggggtcctcagagccctcttctttaaggtcatcaaggattacccttccaacgaagacctt
cacgaaaggctggaggttttcaaggccctcacagacaatgggagacacatcacctacttggaggaagagctggctgactttgtcctgc
agtggatggatgttggcttgtcctcggaattccttctggtgctggtgaacttggtcaaattcaatagctgttacctcgacgagtacatcgca
aggatggttcagatgatctgtctgctgtgcgtccggaccgcgtcctctgtggacatagaggtctccctgcaggtgctggacgccgtggt
ctgctacaactgcctgccggctgagagcctcccgctgttcatcgttaccctctgtcgcaccatcaacgtcaaggagctctgcgagcctt
gctggaagctgatgcggaacctccttggcacccacctgggccacagcgccatctacaacatgtgccacctcatggaggacagagcct
acatggaggacgcgcccctgctgagaggagccgtgttttttgtgggcatggctctctggggagcccaccggctctattctctcaggaa
ctcgccgacatctgtgttgccatcattttaccaggccatggcatgtccgaacgaggtggtgtcctatgagatcgtcctgtccatcaccag
gctcatcaagaagtataggaaggagctccaggtggtggcgtgggacattctgctgaacatcatcgaacggctccttcagcagctccag
accggatcatccggtagctctggcagctctggcagttctggtagcagcggctccgcaagccacgccacgcgtgtgtatgagatgc
tggtcagccacattcagctccactacaagcacagctacaccctgccaatcgcgagcagcatccggctgcaggcctttgacttcctgttg
ctgctgcgggccgactcactgcaccgcctgggcctgcccaacaaggatggagtcgtgcggttcagcccctactgcgtctgcgggtc
aggctccctgctcttccgcgtcctgctgcagtgcttgaagcaggagtctgactggaaggtgctgaagctggttctgggcaggctgcct
gagtccctgcgctataaagtgctcatctttacttccccttgcagtgtggaccagctgtgctctgctctctgctccatgctttcaggcccaaa
gacactggagcggctccgaggcgccccagaaggcttctccagaactgacttgcacctggccgtggttccagtgctgacagcattaat
ctcttaccataactacctggacaaaaccaaacagcgcgagatggtctactgcctggagcagggcctcatccaccgctgtgccagcca
gtgcgtcgtggccttgtccatctgcagcgtggagatgcctgacatcatcatcaaggcgctgcctgttctggtggtgaagctcacgcaca
tctcagccacagccagcatggccgtcccactgctggagttcctgtccactctggccaggctgccgcacctctacaggaactttgccgc
ggagcagtatgccagtgtgttcgccatctccctgccgtacaccaacccctccaagtttaatcagtacatcgtgtgtctggcccatcacgt
catagccatgtggttcatcaggtgccgcctgcccttccggaaggattttgtccctttcatcactaagggcctgcggtccaatgtcctcttgt
ctggctccgggaaaaacctccacctggagctcacggaaacctgtctggacatgatggctcgatacgtcttctccaacttcacggctgtc
ccgaagaggtctcctgtgggcgagttcctcctagcgggtggcaggaccaaaacctggctggttgggaacaagcttgtcactgtgacg
acaagcgtgggaaccgggacccggtcgttactaggctctagcggcagcctggcggcctatgtgcccctgctgacccagggctggg
cggagatcctggtccggaggcccacagggaacaccagctggctgatgagcctggagaacccgggcagcggcatcaaccccagttt
cgtgttcctgcagctctaccattcccccttctttggcgacgagtcaaacaagccaatcctgctgcccaatgagtcacagtcctttgagcg
gtcggtgcagctcctcgaccagatcccatcatacgacacccacaagatcgccgtcctgtatgttggagaaggccagagcaacagcga
gctcgccatcctgtccaatgagcatggctcctacaggtacacggagttcctgacgggcctgggccggctcatcgagctgaaggactg
ccagccggacaaggtgtacctgggaggcctggacgtgtgtggtgaggacggccagttcacctactgctggcacgatgacatcatgc
aagccgtcttccacatcgccaccctgatgcccaccaaggacgtggacaagcaccgctgcgacaagaagcgccacctgggcaacga
ctttgtgtccattgtctacaatgactccggtgaggacttcaagcttggcaccatcaagggccagttcaactttgtccacgtgatcgtcacc
ccgctggactacgagtgcaacctggtgtccctgcagtgcaggaaagacatggagggccttgtggacaccagcgtggccaagatcgt
gtctgaccgcaacctgcccttcgtggcccgccagatggccctgcacgcaaatatggcctcacaggtgcatcatagccgctccaaccc
caccgatatctacccctccaagtggattgcccggctccgccacatcaagggctccgccagcggatctgcgaggaagccgcctactc
caaccccagctga
SEQ ID NO: 23, DNA, Artificial sequence, Micro-Tuberin 6 codon-optimized coding
sequence
atggaatgcggcctgaataatagaatcaggatgatcggccagatttgcgaggtggcaaagacaaagaagttcgaggagcacgcagt
ggaggctctgtggaaagccgtggccgacctgctgcagccagagaggcccctggaggccaggcacgccgtgctggccctgctgaa
agccatcgtgcagggccagggcgagaggctgggggtgctgagggcccttttcttcaaagtgattaaggactacccttctaacgaggat
ctgcatgagcggctggaggtgttcaaggccctgaccgacaacggacgccatatcacctacctggaggaggaactggccgacttcgt
gctccagtggatggacgtgggactgtcctctgagtttctgctggtgctggtgaatctggtgaagttcaatagctgttacctggatgagtac
atcgctagaatggtgcagatgatctgcctgctgtgcgtgagaaccgctagtagcgtcgatatcgaagtgtctctgcaggtgctggacgc
cgtggtctgctacaattgtctccccgccgagagcctgcctctgttcattgtgacactgtgccggaccatcaatgtgaaagaactgtgtga
accatgctggaagctgatgagaaatctgctgggcacccacctgggccactctgccatctataacatgtgtcacctgatggaggacaga
gcatatatggaagacgcccctttgctgcggggcgccgtgttcttcgtgggcatggccctgtggggggctcacaggctgtattcactgc
gcaattcacccacctccgtgctgcctagcttttaccaggccatggcctgccccaacgaagtggtgagctacgaaatcgtgctgagcatt
acgcgcctgattaagaaataccggaaggaactgcaggtggtggcctgggacatcctgctgaacattatcgagcggctgctgcagcag
ctgcagacaggatcatccggtagctctggcagctctggcagttctggtagcagcggctccgcctcccatgccacacgcgtctacg
aaatgctggtgagccacatccagctgcactataagcacagctacacactgcccatcgcttcctccatcagactgcaggccttcgatttcc
tgctgctgttgcgggctgactcactgcataggctggggctgcccaataaggatggcgtggtgagattcagcccatactgtgtgtgcgg
gtcaggctcactgctctttcgggtgctgctgcagtgcctgaaacaggagagcgactggaaagtcctgaaactggtgctgggccgcct
gcccgagagcctgcgatacaaggtgctgatcttcacatccccttgtagcgtggaccagctgtgcagcgccctgtgtagcatgctgtctg
gcccaaagaccctcgaacggttgcgcggcgccccagagggcttctcccgcactgacctgcacctggctgtggtgcctgtgttgaccg
ccctcattagctaccacaattatctcgacaagaccaagcagagagagatggtgtattgcctggagcagggcctgattcaccggtgcgc
ttctcaatgcgtggtcgccctgagtatttgcagcgtggagatgccagacattattatcaaggccctgcctgtgcttgtggtgaaactgact
cacattagcgccacagctagcatggctgtgcctctgttggaattcctgtccacactggccagactgccccacctgtataggaactttgca
gccgagcagtacgcctcagtgttcgccatttccctgccttacaccaatccttctaagtttaaccagtatatgtgtgcctggcccaccacg
tgattgccatgtggttcatcaggtgcaggctgcctttccggaaggacttcgtccctttcattaccaagggtctgaggtccaatgtgctgct
gtcaggctccgggaagaacctgcacctcgagctgactgaaacctgcctggacatgatggccaggtacgtgtttagtaacttcactgct
gtgcccaaacggtcacctgtgggggagtttctgctggccggcgggcgcacaaagacctggctggtggggaataagctggtgaccgt
gacaaccagtgtgggcaccggcaccagatctttactgggctctagcggcagcctggccgcctatgtgcccctgctgactcagggctg
ggccgaaattctggtgagacggcccaccggcaataccagctggctgatgagcctggagaaccctggcagcggcataaaccctagct
tcgtgttcctgcagctgtaccacagccctttctttggcgacgagtctaataagcccatcctgctgccaaacgagtcccagagcttcgagc
gctctgtgcagctgctggaccagatcccctcttacgatacccacaaaatcgccgtgctgtacgtgggcgaaggccagtctaactccga
gctggccatcctgtctaacgagcacggcagttacagatatactgagtttctgacagggctgggccgcctgattgagctcaaagactgcc
agcccgacaaggtgtacctgggaggactggacgtgtgcggcgaggacggacagttcacatattgctggcacgatgatatcatgcag
gccgtgtttcacattgccacattgatgcccaccaaggatgtggacaaacacaggtgcgacaagaagcgccacctgggcaacgacttc
gtgtctatcgtgtacaacgattccggggaagattttaaactcgggacaatcaagggccagtttaacttcgtgcacgtgatcgtgacccca
ctggactacgagtgcaatctggtgtctctgcagtgcagaaaggatatggagggtctggtggataccagcgtcgccaagattgtgtccg
atcgcaacctgccattcgtggcccggcagatggccctgcacgccaacatggcctcccaggtgcaccactctagatccaacccaacgg
acatctatccttccaaatggatcgcccgcctgcgccacattaagcggctgcgccagcgcatttgcgaggaggccgcctactccaatcc
atcttga
SEQ ID NO: 24, DNA, Artificial sequence, Micro-Tuberin 6 native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGGAATGTGGCCTCAACAATCGCATCCGGATGATAGGGCAGATTTGT
GAAGTCGCAAAAACCAAGAAATTTGAAGAGCACGCAGTGGAAGCACTCTGGAA
GGCGGTCGCGGATCTGTTGCAGCCGGAGCGGCCGCTGGAGGCCCGGCACGCGGT
GCTGGCTCTGCTGAAGGCCATCGTGCAGGGGCAGGGCGAGCGTTTGGGGGTCCT
CAGAGCCCTCTTCTTTAAGGTCATCAAGGATTACCCTTCCAACGAAGACCTTCAC
GAAAGGCTGGAGGTTTTCAAGGCCCTCACAGACAATGGGAGACACATCACCTAC
TTGGAGGAAGAGCTGGCTGACTTTGTCCTGCAGTGGATGGATGTTGGCTTGTCCT
CGGAATTCCTTCTGGTGCTGGTGAACTTGGTCAAATTCAATAGCTGTTACCTCGA
CGAGTACATCGCAAGGATGGTTCAGATGATCTGTCTGCTGTGCGTCCGGACCGCG
TCCTCTGTGGACATAGAGGTCTCCCTGCAGGTGCTGGACGCCGTGGTCTGCTACA
ACTGCCTGCCGGCTGAGAGCCTCCCGCTGTTCATCGTTACCCTCTGTCGCACCAT
CAACGTCAAGGAGCTCTGCGAGCCTTGCTGGAAGCTGATGCGGAACCTCCTTGG
CACCCACCTGGGCCACAGCGCCATCTACAACATGTGCCACCTCATGGAGGACAG
AGCCTACATGGAGGACGCGCCCCTGCTGAGAGGAGCCGTGTTTTTTGTGGGCATG
GCTCTCTGGGGAGCCCACCGGCTCTATTCTCTCAGGAACTCGCCGACATCTGTGT
TGCCATCATTTTACCAGGCCATGGCATGTCCGAACGAGGTGGTGTCCTATGAGAT
CGTCCTGTCCATCACCAGGCTCATCAAGAAGTATAGGAAGGAGCTCCAGGTGGT
GGCGTGGGACATTCTGCTGAACATCATCGAACGGCTCCTTCAGCAGCTCCAGACC
GGATCATCCGGTAGCTCTGGCAGCTCTGGCAGTTCTGGTAGCAGCGGCTCC
GCAAGCCACGCCACGCGTGTGTATGAGATGCTGGTCAGCCACATTCAGCTCCACT
ACAAGCACAGCTACACCCTGCCAATCGCGAGCAGCATCCGGCTGCAGGCCTTTG
ACTTCCTGTTGCTGCTGCGGGCCGACTCACTGCACCGCCTGGGCCTGCCCAACAA
GGATGGAGTCGTGCGGTTCAGCCCCTACTGCGTCTGCGGGTCAGGCTCCCTGCT
CTTCCGCGTCCTGCTGCAGTGCTTGAAGCAGGAGTCTGACTGGAAGGTGCTGAAG
CTGGTTCTGGGCAGGCTGCCTGAGTCCCTGCGCTATAAAGTGCTCATCTTTACTTC
CCCTTGCAGTGTGGACCAGCTGTGCTCTGCTCTCTGCTCCATGCTTTCAGGCCCAA
AGACACTGGAGCGGCTCCGAGGCGCCCCAGAAGGCTTCTCCAGAACTGACTTGC
ACCTGGCCGTGGTTCCAGTGCTGACAGCATTAATCTCTTACCATAACTACCTGGA
CAAAACCAAACAGCGCGAGATGGTCTACTGCCTGGAGCAGGGCCTCATCCACCG
CTGTGCCAGCCAGTGCGTCGTGGCCTTGTCCATCTGCAGCGTGGAGATGCCTGAC
ATCATCATCAAGGCGCTGCCTGTTCTGGTGGTGAAGCTCACGCACATCTCAGCCA
CAGCCAGCATGGCCGTCCCACTGCTGGAGTTCCTGTCCACTCTGGCCAGGCTGCC
GCACCTCTACAGGAACTTTGCCGCGGAGCAGTATGCCAGTGTGTTCGCCATCTCC
CTGCCGTACACCAACCCCTCCAAGTTTAATCAGTACATCGTGTGTCTGGCCCATC
ACGTCATAGCCATGTGGTTCATCAGGTGCCGCCTGCCCTTCCGGAAGGATTTTGT
CCCTTTCATCACTAAGGGCCTGCGGTCCAATGTCCTCTTGTCTGGCTCCGGGAAA
AACCTCCACCTGGAGCTCACGGAAACCTGTCTGGACATGATGGCTCGATACGTCT
TCTCCAACTTCACGGCTGTCCCGAAGAGGTCTCCTGTGGGCGAGTTCCTCCTAGC
GGGTGGCAGGACCAAAACCTGGCTGGTTGGGAACAAGCTTGTCACTGTGACGAC
AAGCGTGGGAACCGGGACCCGGTCGTTACTAGGCTCTAGCGGCAGCCTGGCGG
CCTATGTGCCCCTGCTGACCCAGGGCTGGGCGGAGATCCTGGTCCGGAGGCCCA
CAGGGAACACCAGCTGGCTGATGAGCCTGGAGAACCCGGGCAGCGGCATCAAC
CCCAGTTTCGTGTTCCTGCAGCTCTACCATTCCCCCTTCTTTGGCGACGAGTCAAA
CAAGCCAATCCTGCTGCCCAATGAGTCACAGTCCTTTGAGCGGTCGGTGCAGCTC
CTCGACCAGATCCCATCATACGACACCCACAAGATCGCCGTCCTGTATGTTGGAG
AAGGCCAGAGCAACAGCGAGCTCGCCATCCTGTCCAATGAGCATGGCTCCTACA
GGTACACGGAGTTCCTGACGGGCCTGGGCCGGCTCATCGAGCTGAAGGACTGCC
AGCCGGACAAGGTGTACCTGGGAGGCCTGGACGTGTGTGGTGAGGACGGCCAGT
TCACCTACTGCTGGCACGATGACATCATGCAAGCCGTCTTCCACATCGCCACCCT
GATGCCCACCAAGGACGTGGACAAGCACCGCTGCGACAAGAAGCGCCACCTGGG
CAACGACTTTGTGTCCATTGTCTACAATGACTCCGGTGAGGACTTCAAGCTTGGC
ACCATCAAGGGCCAGTTCAACTTTGTCCACGTGATCGTCACCCCGCTGGACTACG
AGTGCAACCTGGTGTCCCTGCAGTGCAGGAAAGACATGGAGGGCCTTGTGGACA
CCAGCGTGGCCAAGATCGTGTCTGACCGCAACCTGCCCTTCGTGGCCCGCCAGAT
GGCCCTGCACGCAAATATGGCCTCACAGGTGCATCATAGCCGCTCCAACCCCACC
GATATCTACCCCTCCAAGTGGATTGCCCGGCTCCGCCACATCAAGCGGCTCCGCC
AGCGGATCTGCGAGGAAGCCGCCTACTCCAACCCCAGCTGAGGCCGGGGCCCTC
CCTCCTGCACTGGCCTTGGACGGTATTGCCTGTCAGTGAAATAAATAAAGTCCTG
ACCCCAGTGCACAGACATAGAGGCACAGATTGCAGTCAGACAGCTCTTTTATTG
ACTTTGTCTGCTTGGTGCGGGGGTTGGGGGGGTGTCGAGGCTCTAGAAGCGGCC
ATGCCCACAGAAGTGGTACACAGAAGCAGGCACAGCCAGCTCCGAGGGCacccagct
ttcttgtacaaagtgggaattcctagagctcgctgatcagcctcgactgtgccttctagttgccagccatctgttgtttgcccctccccc
gtgccttccttgaccctggaaggtgccactcccactgtcctttcctaataaaatgaggaaattgcatcgcattgtctgagtaggtg
tcattctattctggggggtggggtggggcaggacagcaagggggaggattgggaagagaatagcaggcatgctggggagg
gccgcaggaacccctagtgatggagttggccactccctctctgcgcgctcgctcgctcactgaggccgggcgaccaaaggtcgccc
gacgcccgggctttgcccgggcggcctcagtgagcgagcgagcgcgcag
SEQ ID NO: 25, DNA, Artificial sequence, Micro-Tuberin 6 codon-optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGGAATGCGGCCTGAATAATAGAATCAGGATGATCGGCCAGATTTGC
GAGGTGGCAAAGACAAAGAAGTTCGAGGAGCACGCAGTGGAGGCTCTGTGGAA
AGCCGTGGCCGACCTGCTGCAGCCAGAGAGGCCCCTGGAGGCCAGGCACGCCGT
GCTGGCCCTGCTGAAAGCCATCGTGCAGGGCCAGGGCGAGAGGCTGGGGGTGCT
GAGGGCCCTTTTCTTCAAAGTGATTAAGGACTACCCTTCTAACGAGGATCTGCAT
GAGCGGCTGGAGGTGTTCAAGGCCCTGACCGACAACGGACGCCATATCACCTAC
CTGGAGGAGGAACTGGCCGACTTCGTGCTCCAGTGGATGGACGTGGGACTGTCC
TCTGAGTTTCTGCTGGTGCTGGTGAATCTGGTGAAGTTCAATAGCTGTTACCTGG
ATGAGTACATCGCTAGAATGGTGCAGATGATCTGCCTGCTGTGCGTGAGAACCG
CTAGTAGCGTCGATATCGAAGTGTCTCTGCAGGTGCTGGACGCCGTGGTCTGCTA
CAATTGTCTCCCCGCCGAGAGCCTGCCTCTGTTCATTGTGACACTGTGCCGGACC
ATCAATGTGAAAGAACTGTGTGAACCATGCTGGAAGCTGATGAGAAATCTGCTG
GGCACCCACCTGGGCCACTCTGCCATCTATAACATGTGTCACCTGATGGAGGACA
GAGCATATATGGAAGACGCCCCTTTGCTGCGGGGCGCCGTGTTCTTCGTGGGCAT
GGCCCTGTGGGGGGCTCACAGGCTGTATTCACTGCGCAATTCACCCACCTCCGTG
CTGCCTAGCTTTTACCAGGCCATGGCCTGCCCCAACGAAGTGGTGAGCTACGAAA
TCGTGCTGAGCATTACGCGCCTGATTAAGAAATACCGGAAGGAACTGCAGGTGG
TGGCCTGGGACATCCTGCTGAACATTATCGAGCGGCTGCTGCAGCAGCTGCAGA
CAGGATCATCCGGTAGCTCTGGCAGCTCTGGCAGTTCTGGTAGCAGCGGCT
CCGCCTCCCATGCCACACGCGTCTACGAAATGCTGGTGAGCCACATCCAGCTGC
ACTATAAGCACAGCTACACACTGCCCATCGCTTCCTCCATCAGACTGCAGGCCTT
CGATTTCCTGCTGCTGTTGCGGGCTGACTCACTGCATAGGCTGGGGCTGCCCAAT
AAGGATGGCGTGGTGAGATTCAGCCCATACTGTGTGTGCGGGTCAGGCTCACTG
CTCTTTCGGGTGCTGCTGCAGTGCCTGAAACAGGAGAGCGACTGGAAAGTCCTG
AAACTGGTGCTGGGCCGCCTGCCCGAGAGCCTGCGATACAAGGTGCTGATCTTC
ACATCCCCTTGTAGCGTGGACCAGCTGTGCAGCGCCCTGTGTAGCATGCTGTCTG
GCCCAAAGACCCTCGAACGGTTGCGCGGCGCCCCAGAGGGCTTCTCCCGCACTG
ACCTGCACCTGGCTGTGGTGCCTGTGTTGACCGCCCTCATTAGCTACCACAATTA
TCTCGACAAGACCAAGCAGAGAGAGATGGTGTATTGCCTGGAGCAGGGCCTGAT
TCACCGGTGCGCTTCTCAATGCGTGGTCGCCCTGAGTATTTGCAGCGTGGAGATG
CCAGACATTATTATCAAGGCCCTGCCTGTGCTTGTGGTGAAACTGACTCACATTA
GCGCCACAGCTAGCATGGCTGTGCCTCTGTTGGAATTCCTGTCCACACTGGCCAG
ACTGCCCCACCTGTATAGGAACTTTGCAGCCGAGCAGTACGCCTCAGTGTTCGCC
ATTTCCCTGCCTTACACCAATCCTTCTAAGTTTAACCAGTATATCGTGTGCCTGGC
CCACCACGTGATTGCCATGTGGTTCATCAGGTGCAGGCTGCCTTTCCGGAAGGAC
TTCGTCCCTTTCATTACCAAGGGTCTGAGGTCCAATGTGCTGCTGTCAGGCTCCG
GGAAGAACCTGCACCTCGAGCTGACTGAAACCTGCCTGGACATGATGGCCAGGT
ACGTGTTTAGTAACTTCACTGCTGTGCCCAAACGGTCACCTGTGGGGGAGTTTCT
GCTGGCCGGCGGGCGCACAAAGACCTGGCTGGTGGGGAATAAGCTGGTGACCGT
GACAACCAGTGTGGGCACCGGCACCAGATCTTTACTGGGCTCTAGCGGCAGCC
TGGCCGCCTATGTGCCCCTGCTGACTCAGGGCTGGGCCGAAATTCTGGTGAGACG
GCCCACCGGCAATACCAGCTGGCTGATGAGCCTGGAGAACCCTGGCAGCGGCA
TAAACCCTAGCTTCGTGTTCCTGCAGCTGTACCACAGCCCTTTCTTTGGCGACGA
GTCTAATAAGCCCATCCTGCTGCCAAACGAGTCCCAGAGCTTCGAGCGCTCTGTG
CAGCTGCTGGACCAGATCCCCTCTTACGATACCCACAAAATCGCCGTGCTGTACG
TGGGCGAAGGCCAGTCTAACTCCGAGCTGGCCATCCTGTCTAACGAGCACGGCA
GTTACAGATATACTGAGTTTCTGACAGGGCTGGGCCGCCTGATTGAGCTCAAAGA
CTGCCAGCCCGACAAGGTGTACCTGGGAGGACTGGACGTGTGCGGCGAGGACGG
ACAGTTCACATATTGCTGGCACGATGATATCATGCAGGCCGTGTTTCACATTGCC
ACATTGATGCCCACCAAGGATGTGGACAAACACAGGTGCGACAAGAAGCGCCAC
CTGGGCAACGACTTCGTGTCTATCGTGTACAACGATTCCGGGGAAGATTTTAAAC
TCGGGACAATCAAGGGCCAGTTTAACTTCGTGCACGTGATCGTGACCCCACTGGA
CTACGAGTGCAATCTGGTGTCTCTGCAGTGCAGAAAGGATATGGAGGGTCTGGT
GGATACCAGCGTCGCCAAGATTGTGTCCGATCGCAACCTGCCATTCGTGGCCCGG
CAGATGGCCCTGCACGCCAACATGGCCTCCCAGGTGCACCACTCTAGATCCAACC
CAACGGACATCTATCCTTCCAAATGGATCGCCCGCCTGCGCCACATTAAGCGGCT
GCGCCAGCGCATTTGCGAGGAGGCCGCCTACTCCAATCCATCTTGAGGCCGGGG
CCCTCCCTCCTGCACTGGCCTTGGACGGTATTGCCTGTCAGTGAAATAAATAAAG
TCCTGACCCCAGTGCACAGACATAGAGGCACAGATTGCAGTCAGACAGCTCTTTT
ATTGACTTTGTCTGCTTGGTGCGGGGGTTGGGacccagctttcttgtacaaagtgggaattcctagagctc
gctgatcagcctcgactgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattctggggggtggggtggggcaggacag
caagggggaggattgggaagagaatagcaggcatgctggggagggccgcaggaacccctagtgatggagttggccactccctctc
tgcgcgctcgctcgctcactgaggccgggcgaccaaaggtcgcccgacgcccgggctttgcccgggcggcctcagtgagcgagc
gagcgcgcag
SEQ ID NO: 26, Protein, Artificial sequence, Micro-Tuberin 6
MECGLNNRIRMIGQICEVAKTKKFEEHAVEALWKAVADLLQPERPLEARHAVLALL
KAIVQGQGERLGVLRALFFKVIKDYPSNEDLHERLEVFKALTDNGRHITYLEEELAD
FVLQWMDVGLSSEFLLVLVNLVKFNSCYLDEYIARMVQMICLLCVRTASSVDIEVSL
QVLDAVVCYNCLPAESLPLFIVTLCRTINVKELCEPCWKLMRNLLGTHLGHSAIYNM
CHLMEDRAYMEDAPLLRGAVFFVGMALWGAHRLYSLRNSPTSVLPSFYQAMACPN
EVVSYEIVLSITRLIKKYRKELQVVAWDILLNIIERLLQQLQTGSSGSSGSSGSSGSSG
SASHATRVYEMLVSHIQLHYKHSYTLPIASSIRLQAFDFLLLLRADSLHRLGLPNKDG
VVRFSPYCVCGSGSLLFRVLLQCLKQESDWKVLKLVLGRLPESLRYKVLIFTSPCSV
DQLCSALCSMLSGPKTLERLRGAPEGFSRTDLHLAVVPVLTALISYHNYLDKTKQRE
MVYCLEQGLIHRCASQCVVALSICSVEMPDIIIKALPVLVVKLTHISATASMAVPLLEF
LSTLARLPHLYRNFAAEQYASVFAISLPYTNPSKFNQYIVCLAHHVIAMWFIRCRLPF
RKDFVPFITKGLRSNVLLSGSGKNLHLELTETCLDMMARYVFSNFTAVPKRSPVGEF
LLAGGRTKTWLVGNKLVTVTTSVGTGTRSLLGSSGSLAAYVPLLTQGWAEILVRRP
TGNTSWLMSLENPGSGINPSFVFLQLYHSPFFGDESNKPILLPNESQSFERSVQLLDQI
PSYDTHKIAVLYVGEGQSNSELAILSNEHGSYRYTEFLTGLGRLIELKDCQPDKVYLG
GLDVCGEDGQFTYCWHDDIMQAVFHIATLMPTKDVDKHRCDKKRHLGNDFVSIVY
NDSGEDFKLGTIKGQFNFVHVIVTPLDYECNLVSLQCRKDMEGLVDTSVAKIVSDRN
LPFVARQMALHANMASQVHHSRSNPTDIYPSKWIARLRHIKRLRQRICEEAAYSNPS
SEQ ID NO: 27, DNA, Artificial sequence, Micro-Tuberin 7 coding sequence
atgagcgccatttacaacatgtgccacctgatggaggaccgcgcctacatggaagacgctcccctgctgcgcggggccgtgttctttg
tgggcatggctctgtggggcgcccacaggctgtattccctgaggaacagccccacttctgtgctgccaagcttttaccaggccatggc
ctgcccaaatgaggtggtgtcttatgagatcgtgctgagcattaccaggctgatcaagaagtacaggaaggagctgcaggtggtggcc
tgggatattctgctgaacattatcgaaaggctgctccagcagctgcagaccctggactcacccgagctgaggaccatcgtgcacgatc
tgctgaccaccgtagaggagctgtgcgatcagaatgagtttcatgggtcccaggaacggtactttgagctggtggagagatgcgccg
accagaggccagagagcagccttctgaacctgatcagctacagagcccagagcatccaccccgccaaggacggctggattcagaa
cctccaggctctgatggagcggttctttaggtctgaaagcagaggcgccgtgcgcatcaaagtgctggacgtgctgtccttcgtgcttct
tattaatcggcagttctatgaggaggagctgatcaacagcgttgtgatctctcagctgtcccatatccctgaggacaaggaccatcaggt
gaggaagctcgccacccagctgctggttgacttggccgagggctgccatacccatcactttaatagcctgctggatatcatcgagaag
gtgatggcccggtccctcagcccccctccagaactggaggagcgggacgtggccgcctactcagcatccctggaggatgtgaagac
cgccgtgctgggcctgctggtcattctgcagaccaaactgtacaccctgcccgccagccatgctacacgggtgtacgagatgctggtg
agccacatccagctgcactacaagcacagctacaccctgcctattgccagctccatccgcctgcaggccttcgactttctgctgctcctc
cgggccgattcactgcaccggctgggtctgcccaacaaggacggcgtggtgagattttctccttactgcgtgtgcggctccggcagcc
tcctgtttagagtgctgctgcagtgtctcaagcaggaatccgactggaaagtgctgaagctggtgctgggcaggctgcccgaaagcct
gaggtacaaggtcctgatcttcacttcaccctgtagcgtggatcagctgtgcagcgccctgtgcagcatgctgagcgggcctaagact
ctggagagactgagaggcgcccctgagggcttttccagaaccgacctgcacctggccgtggtgccagtgctgaccgccctgatctcc
tatcacaattatctggacaagaccaagcagagagagatggtgtactgcctggagcagggcctgatccatcggtgtgccagccagtgc
gtggtggccctgtccatttgctccgtggaaatgcccgacatcatcatcaaggccctgcccgtgctggtggtgaagctgacccatatctc
cgccaccgccagcatggccgtgcccctgctggaatttctgtctaccctggctcgtctgccacatctgtaccggaattttgcggccgagc
agtacgcatccgtgttcgctatcagcctgccttacacaaatccctccaagttcaatcaatacattgtatgtctggcccaccacgtgatcgct
atgtggttcatcaggtgcaggctgcccttcagaaaggatttcgtgccatttatcaccaagggcctgaggagcaatgtactgctgagcgg
ctcaggaaagaacctgcatctggaactgaccgagacctgtctggacatgatggctcgatacgtgttctccaacttcaccgctgtaccca
agcggtcaccagtgggggaattcctgctggccgggggcaggactaagacatggctggtcggcaacaaactggttactgtgaccacc
tccgtcggcactggcactagaagtctgctgggctccagtggcagcctggccgcctacgtgccactgctgacccaggggtgggccga
gatcctggtgagacgcccaactggcaacacttcatggctgatgtccctggagaaccctggatccggcatcaatccaagcttcgtgttcc
tgcagctttaccactcaccatttttcggagacgagtccaacaagcccattctgctgccaaacgaatcccagtctttcgagagaagcgtgc
agctgctggaccagattcccagctacgacacccacaaaattgccgtgctctacgtgggcgaaggccagagcaactccgagctggcc
attctgtctaacgagcacggatcttacagatacaccgagtttctgaccggactggggcgactgatcgagctgaaggactgtcagcccg
acaaggtatacctgggaggcctggacgtgtgcggcgaggatggccagtttacctattgctggcatgatgacatcatgcaggccgtgtt
ccacatcgctaccctgatgcctacaaaggacgtggataagcataggtgcgataagaagaggcacctgggcaacgacttcgtgtctatc
gtgtacaacgattctggcgaggactttaagctgggtaccattaagggccagtttaatttcgtgcatgtgatcgtgacccctctggattatga
gtgtaatctggtgagcctgcagtgcagaaaggatatggagggactggtggacacctctgtcgcaaaaatcgtgagcgacagaaatct
gcctttcgtggccaggcagatggccctgcatgccaatatggccagtcaggtgcaccacagccggtctaatcccaccgacatctacccc
agtaagtggattgcccgactccggcacatcaagaggctgcgccagcggatttgtgaggaagctgcctatagcaaccctagctga
SEQ ID NO: 28, DNA, Artificial sequence, Micro-Tuberin 7 codon optimized coding
sequence
atgaagtttgaagagcatgccgtggaagccctgtggaaggccgtggctgacctgctgcagcctgaacgtcctctggaagctcgccat
gccgtgttggccctgctgaaggctatcgtgcagggacagggagagaggctgggcgtgctgcgcgccctgttctttaaggtgattaag
gactacccctccaacgaggacctgcacgagaggctggaagtgtttaaggccctgaccgataacggcaggcacatcacttacctgga
ggaggagctggccgacttcgtgctgcagtggatggatgtgggcctgagcagcgaattcctgctggtgctggtgaatctggtgaaattc
aacagctgttacctggatgaatacattgccagaatggtccagatgatctgcctgctttgcgtgcgaacagcctcatcagtggatatcgag
gtgtctctgcaggtgctggacgccgtggtgtgctataactgtctgcccgctgagagcctccctctgtttatcgtgacactgtgccgcacc
atcaacgtgaaagaactgtgcgagccttgctggaaactgatgagaaacctgctggggacacacctgggccacagcgccatctacaac
atgtgccacctcatggaagaccgggcctacatggaggatgcccccctgctgcgcggggccgtgtttttcgtgggaatggctctgtggg
gggcccatagactgtacagcctcaggaattccccaaccagcgtgctgccatccttttatcaggccatggcctgtcccaacgaagtggtg
agctatgagatcgtcctgtccatcacccggctgattaaaaaataccggaaagagctgcaggtggtggcttgggatatcctgctgaatat
catcgagaggctgctgcagcagctgcagactctcgacagccctgagctgcgcaccattgtgcacgatttgctgaccaccgtggaaga
gctgtgcgatcagaacgaattccacggctcccaggagaggtactttgagctggtggaaagatgtgccgaccagaggcctgagtctag
tctcctgaatctgatctcctacagagctcaatccatccatggtggaagcggcggcagtggaggctccaccctgcccgccagccacgc
caccagagtgtacgagatgctggtcagtcacatccagctgcactacaaacattcctacactctgccaatcgccagctcaatccggctgc
aggccttcgactttctgctgctgctgagagccgacagcctgcacagactcggcctgcctaacaaggatggtgtggtgaggttcagtcc
atactgtgtgtgcgggtctggctctctgctgttcagagtcctgctgcagtgcctcaagcaggagagcgactggaaggtcctgaagctg
gtcctgggccgtctgcccgagagtctgaggtacaaggtgctgattttcaccagcccctgcagtgtggaccagctgtgttccgccctctg
cagcatgctgagcggcccaaagaccctggagcggctgaggggcgcccccgagggatttagcaggaccgatctgcacctggccgt
ggtgcccgtgctgactgccctgatctcctaccacaattacctggataagaccaaacagcgggagatggtgtattgcctggagcagggg
ctgatccatcgctgtgcctctcagtgcgtggtggccctgagtatctgcagcgtggaaatgcccgatatcattatcaaggccctgcctgtg
ctggtggtgaagctgacccacatcagcgccaccgccagcatggccgtgcctctcctggagttcctgagtacactggccagactgccc
catctgtatagaaattttgccgcagaacagtacgccagcgtgttcgccatctctctgccatacacaaatccctccaagttcaaccagtata
tcgtgtgtctcgcccaccacgtgatcgcaatgtggttcatccgctgccggcttcccttcaggaaggacttcgtgccctttatcactaaggg
actgaggagtaacgtgctgctcagtggcagcgggaagaacctgcatctcgagctgaccgagacatgcctggacatgatggctaggt
atgtgttctccaacttcacagccgtgcccaaacggtccccagtgggggaatttctgctggcagggggcagaaccaagacatggctggt
ggggaataagctggtgaccgtgaccacctccgtgggcactggcacccggagcctgctgggcgggagcggcggcctggccgccta
cgtgcctctgctgacacagggctgggcagagatcctggtgagaagacctacaggcaacaccagctggctgatgtccctggagaacc
ccggtagtggcattaatccatcttttgtgtttctgcagctgtaccatagccccttcttcggcgatgaaagcaataagcctatcctgctgccc
aacgagagccagtcattcgagaggagcgtgcagctgctcgatcagatccctagctatgacactcacaaaatcgccgtgctgtacgtgg
gcgaggggcagtccaactctgagctggccatcctgagcaatgagcacgggagctacagatataccgagtttctgaccggcctgggc
cggctgattgagctgaaggattgccagcccgacaaagtgtacctgggcggcctggatgtgtgcggggaggacggccagtttacttac
tgttggcacgacgatatcatgcaggccgtgttccacatcgctactctgatgcctacaaaagatgtggacaagcaccggtgcgacaaaa
aaagacatctgggaaatgactttgtgtccattgtgtacaatgactccggcgaggactttaaactgggaaccatcaagggccagttcaatt
tcgtgcacgtgatcgtgactccactggattacgagtgtaatctcgtgagcctgcagtgcaggaaagacatggaagggctggtggatac
ctctgtggccaaaatcgtgtcagacaggaacctgccctttgtggctcggcagatggccctgcacgccaatatggccagtcaggtgcac
cactcccggagcaaccctacagatatctatcccagcaaatggatcgcccgcctgaggcacattaagcgcctgcggcagagaatctgt
gaagaggccgcctacagcaacccatcatgaacccagctttcttgtacaaagtgggaattcctagagctcgctgatcagcctcgactgt
gccttctagttgccagccatctgttgtttgcccctcccccgtgccttccttgaccctggaaggtgccactcccactgtcctttcctaa
taaaatgaggaaattgcatcgcattgtctgagtaggtgtcattctattctggggggtggggtggggcaggacagcaaggggga
ggattgggaagagaatagcaggcatgctggggagggccgc
SEQ ID NO: 29, DNA, Artificial sequence, Micro-Tuberin 7 native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGGGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGAGCGCCATCTACAACATGTGCCACCTCATGGAGGACAGAGCCTAC
ATGGAGGACGCGCCCCTGCTGAGAGGAGCCGTGTTTTTTGTGGGCATGGCTCTCT
GGGGAGCCCACCGGCTCTATTCTCTCAGGAACTCGCCGACATCTGTGTTGCCATC
ATTTTACCAGGCCATGGCATGTCCGAACGAGGTGGTGTCCTATGAGATCGTCCTG
TCCATCACCAGGCTCATCAAGAAGTATAGGAAGGAGCTCCAGGTGGTGGCGTGG
GACATTCTGCTGAACATCATCGAACGGCTCCTTCAGCAGCTCCAGACCTTGGACA
GCCCGGAGCTCAGGACCATCGTCCATGACCTGTTGACCACGGTGGAGGAGCTGT
GTGACCAGAACGAGTTCCACGGGTCTCAGGAGAGATACTTTGAACTGGTGGAGA
GATGTGCGGACCAGAGGCCTGAGTCCTCCCTCCTGAACCTGATCTCCTATAGAGC
GCAGTCCATCCACCCGGCCAAGGACGGCTGGATTCAGAACCTGCAGGCGCTGAT
GGAGAGATTCTTCAGGAGCGAGTCCCGAGGCGCCGTGCGCATCAAGGTGCTGGA
CGTGCTGTCCTTTGTGCTGCTCATCAACAGGCAGTTCTATGAGGAGGAGCTGATT
AACTCAGTGGTCATCTCGCAGCTCTCCCACATCCCCGAGGATAAAGACCACCAG
GTCCGAAAGCTGGCCACCCAGTTGCTGGTGGACCTGGCAGAGGGCTGCCACACA
CACCACTTCAACAGCCTGCTGGACATCATCGAGAAGGTGATGGCCCGCTCCCTCT
CCCCACCCCCGGAGCTGGAAGAAAGGGATGTGGCCGCATACTCGGCCTCCTTGG
AGGATGTGAAGACAGCCGTCCTGGGGCTTCTGGTCATCCTTCAGACCAAGCTGTA
CACCCTGCCTGCAAGCCACGCCACGCGTGTGTATGAGATGCTGGTCAGCCACATT
CAGCTCCACTACAAGCACAGCTACACCCTGCCAATCGCGAGCAGCATCCGGCTG
CAGGCCTTTGACTTCCTGTTGCTGCTGCGGGCCGACTCACTGCACCGCCTGGGCC
TGCCCAACAAGGATGGAGTCGTGCGGTTCAGCCCCTACTGCGTCTGCGGCTCCG
GCTCCCTGCTCTTCCGCGTCCTGCTGCAGTGCTTGAAGCAGGAGTCTGACTGGAA
GGTGCTGAAGCTGGTTCTGGGCAGGCTGCCTGAGTCCCTGCGCTATAAAGTGCTC
ATCTTTACTTCCCCTTGCAGTGTGGACCAGCTGTGCTCTGCTCTCTGCTCCATGCT
TTCAGGCCCAAAGACACTGGAGCGGCTCCGAGGCGCCCCAGAAGGCTTCTCCAG
AACTGACTTGCACCTGGCCGTGGTTCCAGTGCTGACAGCATTAATCTCTTACCAT
AACTACCTGGACAAAACCAAACAGCGCGAGATGGTCTACTGCCTGGAGCAGGGC
CTCATCCACCGCTGTGCCAGCCAGTGCGTCGTGGCCTTGTCCATCTGCAGCGTGG
AGATGCCTGACATCATCATCAAGGCGCTGCCTGTTCTGGTGGTGAAGCTCACGCA
CATCTCAGCCACAGCCAGCATGGCCGTCCCACTGCTGGAGTTCCTGTCCACTCTG
GCCAGGCTGCCGCACCTCTACAGGAACTTTGCCGCGGAGCAGTATGCCAGTGTGT
TCGCCATCTCCCTGCCGTACACCAACCCCTCCAAGTTTAATCAGTACATCGTGTG
TCTGGCCCATCACGTCATAGCCATGTGGTTCATCAGGTGCCGCCTGCCCTTCCGG
AAGGATTTTGTCCCTTTCATCACTAAGGGCCTGCGGTCCAATGTCCTCTTGTCTG
GCTCAGGAAAAAACCTCCACCTGGAGCTCACGGAAACCTGTCTGGACATGATGG
CTCGATACGTCTTCTCCAACTTCACGGCTGTCCCGAAGAGGTCTCCTGTGGGCGA
GTTCCTCCTAGCGGGTGGCAGGACCAAAACCTGGCTGGTTGGGAACAAGCTTGT
CACTGTGACGACAAGCGTGGGAACCGGGACCCGGTCGTTACTAGGCTCCAGTG
GCAGCCTGGCGGCCTATGTGCCCCTGCTGACCCAGGGCTGGGCGGAGATCCTGG
TCCGGAGGCCCACAGGGAACACCAGCTGGCTGATGAGCCTGGAGAACCCGGGA
TCCGGCATCAACCCCAGTTTCGTGTTCCTGCAGCTCTACCATTCCCCCTTCTTTGG
CGACGAGTCAAACAAGCCAATCCTGCTGCCCAATGAGTCACAGTCCTTTGAGCG
GTCGGTGCAGCTCCTCGACCAGATCCCATCATACGACACCCACAAGATCGCCGTC
CTGTATGTTGGAGAAGGCCAGAGCAACAGCGAGCTCGCCATCCTGTCCAATGAG
CATGGCTCCTACAGGTACACGGAGTTCCTGACGGGCCTGGGCCGGCTCATCGAG
CTGAAGGACTGCCAGCCGGACAAGGTGTACCTGGGAGGCCTGGACGTGTGTGGT
GAGGACGGCCAGTTCACCTACTGCTGGCACGATGACATCATGCAAGCCGTCTTCC
ACATCGCCACCCTGATGCCCACCAAGGACGTGGACAAGCACCGCTGCGACAAGA
AGCGCCACCTGGGCAACGACTTTGTGTCCATTGTCTACAATGACTCCGGTGAGGA
CTTCAAGCTTGGCACCATCAAGGGCCAGTTCAACTTTGTCCACGTGATCGTCACC
CCGCTGGACTACGAGTGCAACCTGGTGTCCCTGCAGTGCAGGAAAGACATGGAG
GGCCTTGTGGACACCAGCGTGGCCAAGATCGTGTCTGACCGCAACCTGCCCTTCG
TGGCCCGCCAGATGGCCCTGCACGCAAATATGGCCTCACAGGTGCATCATAGCC
GCTCCAACCCCACCGATATCTACCCCTCCAAGTGGATTGCCCGGCTCCGCCACAT
CAAGCGGCTCCGCCAGCGGATCTGCGAGGAAGCCGCCTACTCCAACCCCAGCTG
AGGCCGGGGCCCTCCCTCCTGCACTGGCCTTGGACGGTATTGCCTGTCAGTGAAA
TAAATAAAGTCCTGACCCCAGTGCACAGACATAGAGGCACAGATTGCAGTCAGA
CAGCTCTTTTATTGACTTTGTCTGCTTGGTGCGGGGGTTGGGGGGGTGTCGAGGC
TCTAGAAGCGGCCATGCCCACAGAAGTGGTACACAGAAGCAGGCACAGCCAGCT
CCGAGGGCacccagctttcttgtacaaagtgggaattcctagagctcgctgatcagcctcgactgtgccttctagttgccagcc
atctgttgtttgcccctcccccgtgccttccttgaccctggaaggtgccactcccactgtcctttcctaataaaatgaggaaattgc
atcgcattgtctgagtaggtgtcattctattctggggggtggggtggggcaggacagcaagggggaggattgggaagagaat
agcaggcatgctggggagggccgcaggaacccctagtgatggagttggccactccctctctgcgcgctcgctcgctcactgaggc
cgggcgaccaaaggtcgcccgacgcccgggctttgcccgggcggcctcagtgagcgagcgagcgcgcag
SEQ ID NO: 30, DNA, Artificial sequence, Micro-Tuberin 7 codon optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGGGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGAGCGCCATTTACAACATGTGCCACCTGATGGAGGACCGCGCCTAC
ATGGAAGACGCTCCCCTGCTGCGCGGGGCCGTGTTCTTTGTGGGCATGGCTCTGT
GGGGCGCCCACAGGCTGTATTCCCTGAGGAACAGCCCCACTTCTGTGCTGCCAAG
CTTTTACCAGGCCATGGCCTGCCCAAATGAGGTGGTGTCTTATGAGATCGTGCTG
AGCATTACCAGGCTGATCAAGAAGTACAGGAAGGAGCTGCAGGTGGTGGCCTGG
GATATTCTGCTGAACATTATCGAAAGGCTGCTCCAGCAGCTGCAGACCCTGGACT
CACCCGAGCTGAGGACCATCGTGCACGATCTGCTGACCACCGTAGAGGAGCTGT
GCGATCAGAATGAGTTTCATGGGTCCCAGGAACGGTACTTTGAGCTGGTGGAGA
GATGCGCCGACCAGAGGCCAGAGAGCAGCCTTCTGAACCTGATCAGCTACAGAG
CCCAGAGCATCCACCCCGCCAAGGACGGCTGGATTCAGAACCTCCAGGCTCTGA
TGGAGCGGTTCTTTAGGTCTGAAAGCAGAGGCGCCGTGCGCATCAAAGTGCTGG
ACGTGCTGTCCTTCGTGCTTCTTATTAATCGGCAGTTCTATGAGGAGGAGCTGAT
CAACAGCGTTGTGATCTCTCAGCTGTCCCATATCCCTGAGGACAAGGACCATCAG
GTGAGGAAGCTCGCCACCCAGCTGCTGGTTGACTTGGCCGAGGGCTGCCATACC
CATCACTTTAATAGCCTGCTGGATATCATCGAGAAGGTGATGGCCCGGTCCCTCA
GCCCCCCTCCAGAACTGGAGGAGCGGGACGTGGCCGCCTACTCAGCATCCCTGG
AGGATGTGAAGACCGCCGTGCTGGGCCTGCTGGTCATTCTGCAGACCAAACTGT
ACACCCTGCCCGCCAGCCATGCTACACGGGTGTACGAGATGCTGGTGAGCCACA
TCCAGCTGCACTACAAGCACAGCTACACCCTGCCTATTGCCAGCTCCATCCGCCT
GCAGGCCTTCGACTTTCTGCTGCTCCTCCGGGCCGATTCACTGCACCGGCTGGGT
CTGCCCAACAAGGACGGCGTGGTGAGATTTTCTCCTTACTGCGTGTGCGGCTCC
GGCAGCCTCCTGTTTAGAGTGCTGCTGCAGTGTCTCAAGCAGGAATCCGACTGG
AAAGTGCTGAAGCTGGTGCTGGGCAGGCTGCCCGAAAGCCTGAGGTACAAGGTC
CTGATCTTCACTTCACCCTGTAGCGTGGATCAGCTGTGCAGCGCCCTGTGCAGCA
TGCTGAGCGGGCCTAAGACTCTGGAGAGACTGAGAGGCGCCCCTGAGGGCTTTT
CCAGAACCGACCTGCACCTGGCCGTGGTGCCAGTGCTGACCGCCCTGATCTCCTA
TCACAATTATCTGGACAAGACCAAGCAGAGAGAGATGGTGTACTGCCTGGAGCA
GGGCCTGATCCATCGGTGTGCCAGCCAGTGCGTGGTGGCCCTGTCCATTTGCTCC
GTGGAAATGCCCGACATCATCATCAAGGCCCTGCCCGTGCTGGTGGTGAAGCTG
ACCCATATCTCCGCCACCGCCAGCATGGCCGTGCCCCTGCTGGAATTTCTGTCTA
CCCTGGCTCGTCTGCCACATCTGTACCGGAATTTTGCGGCCGAGCAGTACGCATC
CGTGTTCGCTATCAGCCTGCCTTACACAAATCCCTCCAAGTTCAATCAATACATT
GTATGTCTGGCCCACCACGTGATCGCTATGTGGTTCATCAGGTGCAGGCTGCCCT
TCAGAAAGGATTTCGTGCCATTTATCACCAAGGGCCTGAGGAGCAATGTACTGCT
GAGCGGCTCAGGAAAGAACCTGCATCTGGAACTGACCGAGACCTGTCTGGACA
TGATGGCTCGATACGTGTTCTCCAACTTCACCGCTGTACCCAAGCGGTCACCAGT
GGGGGAATTCCTGCTGGCCGGGGGCAGGACTAAGACATGGCTGGTCGGCAACAA
ACTGGTTACTGTGACCACCTCCGTCGGCACTGGCACTAGAAGTCTGCTGGGCTC
CAGTGGCAGCCTGGCCGCCTACGTGCCACTGCTGACCCAGGGGTGGGCCGAGAT
CCTGGTGAGACGCCCAACTGGCAACACTTCATGGCTGATGTCCCTGGAGAACCCT
GGATCCGGCATCAATCCAAGCTTCGTGTTCCTGCAGCTTTACCACTCACCATTTT
TCGGAGACGAGTCCAACAAGCCCATTCTGCTGCCAAACGAATCCCAGTCTTTCGA
GAGAAGCGTGCAGCTGCTGGACCAGATTCCCAGCTACGACACCCACAAAATTGC
CGTGCTCTACGTGGGCGAAGGCCAGAGCAACTCCGAGCTGGCCATTCTGTCTAAC
GAGCACGGATCTTACAGATACACCGAGTTTCTGACCGGACTGGGCGACTGATC
GAGCTGAAGGACTGTCAGCCCGACAAGGTATACCTGGGAGGCCTGGACGTGTGC
GGCGAGGATGGCCAGTTTACCTATTGCTGGCATGATGACATCATGCAGGCCGTGT
TCCACATCGCTACCCTGATGCCTACAAAGGACGTGGATAAGCATAGGTGCGATA
AGAAGAGGCACCTGGGCAACGACTTCGTGTCTATCGTGTACAACGATTCTGGCG
AGGACTTTAAGCTGGGTACCATTAAGGGCCAGTTTAATTTCGTGCATGTGATCGT
GACCCCTCTGGATTATGAGTGTAATCTGGTGAGCCTGCAGTGCAGAAAGGATAT
GGAGGGACTGGTGGACACCTCTGTCGCAAAAATCGTGAGCGACAGAAATCTGCC
TTTCGTGGCCAGGCAGATGGCCCTGCATGCCAATATGGCCAGTCAGGTGCACCAC
AGCCGGTCTAATCCCACCGACATCTACCCCAGTAAGTGGATTGCCCGACTCCGGC
ACATCAAGAGGCTGCGCCAGCGGATTTGTGAGGAAGCTGCCTATAGCAACCCTA
GCTGAGGCCGGGGCCCTCCCTCCTGCACTGGCCTTGGACGGTATTGCCTGTCAGT
GAAATAAATAAAGTCCTGACCCCAGTGCACAGACATAGAGGCACAGATTGCAGT
CAGACAGCTCTTTTATTGACTTTGTCTGCTTGGTGCGGGGGTTGGGGGGGTGTCG
AGGCTCTAGAAGCGGCCATGCCCACAGAAGTGGTACACAGAAGCAGGCACAGCC
AGCTCCGAGGGCacccagctttcttgtacaaagtgggaattcctagagctcgctgatcagcctcgactgtgccttctagttg
ccagccatctgttgtttgcccctcccccgtgccttccttgaccctggaaggtgccactcccactgtcctttcctaataaaatgagg
aaattgcatcgcattgtctgagtaggtgtcattctattctggggggtggggtggggcaggacagcaagggggaggattgggaa
gagaatagcaggcatgctggggagggccgcaggaacccctagtgatggagttggccactccctctctgcgcgctcgctcgctcac
tgaggccgggcgaccaaaggtcgcccgacgcccgggctttgcccgggggcctcagtgagcgagcgagcgcgcag
SEQ ID NO: 31, Protein, Artificial sequence, Micro-Tuberin 7 protein sequence
MSAIYNMCHLMEDRAYMEDAPLLRGAVFFVGMALWGAHRLYSLRNSPTSVLPSFY
QAMACPNEVVSYEIVLSITRLIKKYRKELQVVAWDILLNIIERLLQQLQTLDSPELRTI
VHDLLTTVEELCDQNEFHGSQERYFELVERCADQRPESSLLNLISYRAQSIHPAKDG
WIQNLQALMERFFRSESRGAVRIKVLDVLSFVLLINRQFYEEELINSVVISQLSHIPED
KDHQVRKLATQLLVDLAEGCHTHHFNSLLDIIEKVMARSLSPPPELEERDVAAYSAS
LEDVKTAVLGLLVILQTKLYTLPASHATRVYEMLVSHIQLHYKHSYTLPIASSIRLQA
FDFLLLLRADSLHRLGLPNKDGVVRFSPYCVCGSGSLLFRVLLQCLKQESDWKVLK
LVLGRLPESLRYKVLIFTSPCSVDQLCSALCSMLSGPKTLERLRGAPEGFSRTDLHLA
VVPVLTALISYHNYLDKTKQREMVYCLEQGLIHRCASQCVVALSICSVEMPDIIIKAL
PVLVVKLTHISATASMAVPLLEFLSTLARLPHLYRNFAAEQYASVFAISLPYTNPSKF
NQYIVCLAHHVIAMWFIRCRLPFRKDFVPFITKGLRSNVLLSGSGKNLHLELTETCLD
MMARYVFSNFTAVPKRSPVGEFLLAGGRTKTWLVGNKLVTVTTSVGTGTRSLLGSS
GSLAAYVPLLTQGWAEILVRRPTGNTSWLMSLENPGSGINPSFVFLQLYHSPFFGDES
NKPILLPNESQSFERSVQLLDQIPSYDTHKIAVLYVGEGQSNSELAILSNEHGSYRYTE
FLTGLGRLIELKDCQPDKVYLGGLDVCGEDGQFTYCWHDDIMQAVFHIATLMPTKD
VDKHRCDKKRHLGNDFVSIVYNDSGEDFKLGTIKGQFNFVHVIVTPLDYECNLVSLQ
CRKDMEGLVDTSVAKIVSDRNLPFVARQMALHANMASQVHHSRSNPTDIYPSKWIA
RLRHIKRLRQRICEEAAYSNPS
SEQ ID NO: 32, DNA, Artificial sequence, Micro-Tuberin 8 coding sequence
atgaaatttgaagagcacgcagtggaagcactctggaaggcggtcgcggatctgttgcagccggagcggccgctggaggcccggc
acgcggtgctggctctgctgaaggccatcgtgcaggggcagggcgagcgtttgggggtcctcagagccctcttctttaaggtcatcaa
ggattacccttccaacgaagaccttcacgaaaggctggaggttttcaaggccctcacagacaatgggagacacatcacctacttggag
gaagagctggctgactttgtcctgcagtggatggatgttggcttgtcctcggaattccttctggtgctggtgaacttggtcaaattcaatag
ctgttacctcgacgagtacatcgcaaggatggttcagatgatctgtctgctgtgcgtccggaccgcgtcctctgtggacatagaggtctc
cctgcaggtgctggacgccgtggtctgctacaactgcctgccggctgagagcctcccgctgttcatcgttaccctctgtcgcaccatca
acgtcaaggagctctgcgagccttgctggaagctgatgcggaacctccttggcacccacctgggccacagcgccatctacaacatgt
gccacctcatggaggacagagcctacatggaggacgcgcccctgctgagaggagccgtgttttttgtgggcatggctctctggggag
cccaccggctctattctctcaggaactcgccgacatctgtgttgccatcattttaccaggccatggcatgtccgaacgaggtggtgtccta
tgagatcgtcctgtccatcaccaggctcatcaagaagtataggaaggagctccaggtggtggcgtgggacattctgctgaacatcatc
gaacggctccttcagcagctccagaccttggacagcccggagctcaggaccatcgtccatgacctgttgaccacggtggaggagctg
tgtgaccagaacgagttccacgggtctcaggagagatactttgaactggtggagagatgtgcggaccagaggcctgagtcctccctc
ctgaacctgatctcctatagagcgcagtccatccacggtggaagcggcggcagtggaggctccaccctgcctgcaagccacgcca
cgcgtgtgtatgagatgctggtcagccacattcagctccactacaagcacagctacaccctgccaatcgcgagcagcatccggctgca
ggcctttgacttcctgttgctgctgcgggccgactcactgcaccgcctgggcctgcccaacaaggatggagtcgtgcggttcagcccc
tactgcgtctgcgggtctggctccctgctcttccgcgtcctgctgcagtgcttgaagcaggagtctgactggaaggtgctgaagctggtt
ctgggcaggctgcctgagtccctgcgctataaagtgctcatctttacttccccttgcagtgtggaccagctgtgctctgctctctgctccat
gctttcaggcccaaagacactggagcggctccgaggcgccccagaaggcttctccagaactgacttgcacctggccgtggttccagt
gctgacagcattaatctcttaccataactacctggacaaaaccaaacagcgcgagatggtctactgcctggagcagggcctcatccac
cgctgtgccagccagtgcgtcgtggccttgtccatctgcagcgtggagatgcctgacatcatcatcaaggcgctgcctgttctggtggt
gaagctcacgcacatctcagccacagccagcatggccgtcccactgctggagttcctgtccactctggccaggctgccgcacctctac
aggaactttgccgcggagcagtatgccagtgtgttcgccatctccctgccgtacaccaacccctccaagtttaatcagtacatcgtgtgt
ctggcccatcacgtcatagccatgtggttcatcaggtgccgcctgcccttccggaaggattttgtccctttcatcactaagggcctgcggt
ccaatgtcctcttgtctggcagcgggaaaaacctccacctggagctcacggaaacctgtctggacatgatggctcgatacgtcttctcc
aacttcacggctgtcccgaagaggtctcctgtgggcgagttcctcctagcgggtggcaggaccaaaacctggctggttgggaacaag
cttgtcactgtgacgacaagcgtgggaaccgggacccggtcgttactaggcgggagcggcggcctggcggcctatgtgcccctgct
gacccagggctgggcggagatcctggtccggaggcccacagggaacaccagctggctgatgagcctggagaacccgggtagtg
gcatcaaccccagtttcgtgttcctgcagctctaccattcccccttctttggcgacgagtcaaacaagccaatcctgctgcccaatgagtc
acagtcctttgagcggtcggtgcagctcctcgaccagatcccatcatacgacacccacaagatcgccgtcctgtatgttggagaaggc
cagagcaacagcgagctcgccatcctgtccaatgagcatggctcctacaggtacacggagttcctgacgggcctgggccggctcatc
gagctgaaggactgccagccggacaaggtgtacctgggaggcctggacgtgtgtggtgaggacggccagttcacctactgctggca
cgatgacatcatgcaagccgtcttccacatcgccaccctgatgcccaccaaggacgtggacaagcaccgctgcgacaagaagcgcc
acctgggcaacgactttgtgtccattgtctacaatgactccggtgaggacttcaagcttggcaccatcaagggccagttcaactttgtcca
cgtgatcgtcaccccgctggactacgagtgcaacctggtgtccctgcagtgcaggaaagacatggagggccttgtggacaccagcgt
ggccaagatcgtgtctgaccgcaacctgcccttcgtggcccgccagatggccctgcacgcaaatatggcctcacaggtgcatcatag
ccgctccaaccccaccgatatctacccctccaagtggattgcccggctccgccacatcaagcggctccgccagcggatctgcgagga
agccgcctactccaaccccagctga
SEQ ID NO: 33, DNA, Artificial sequence, Micro-Tuberin 8 codon-optimized
sequence
atgaagtttgaagagcatgccgtggaagccctgtggaaggccgtggctgacctgctgcagcctgaacgtcctctggaagctcgccat
gccgtgttggccctgctgaaggctatcgtgcagggacagggagagaggctgggcgtgctgcgcgccctgttctttaaggtgattaag
gactacccctccaacgaggacctgcacgagaggctggaagtgtttaaggccctgaccgataacggcaggcacatcacttacctgga
ggaggagctggccgacttcgtgctgcagtggatggatgtgggcctgagcagcgaattcctgctggtgctggtgaatctggtgaaattc
aacagctgttacctggatgaatacattgccagaatggtccagatgatctgcctgctttgcgtgcgaacagcctcatcagtggatatcgag
gtgtctctgcaggtgctggacgccgtggtgtgctataactgtctgcccgctgagagcctccctctgtttatcgtgacactgtgccgcacc
atcaacgtgaaagaactgtgcgagccttgctggaaactgatgagaaacctgctggggacacacctgggccacagcgccatctacaac
atgtgccacctcatggaagaccgggcctacatggaggatgcccccctgctgcgcggggccgtgtttttcgtgggaatggctctgtggg
gggcccatagactgtacagcctcaggaattccccaaccagcgtgctgccatccttttatcaggccatggcctgtcccaacgaagtggtg
agctatgagatcgtcctgtccatcacccggctgattaaaaaataccggaaagagctgcaggtggtggcttgggatatcctgctgaatat
catcgagaggctgctgcagcagctgcagactctcgacagccctgagctgcgcaccattgtgcacgatttgctgaccaccgtggaaga
gctgtgcgatcagaacgaattccacggctcccaggagaggtactttgagctggtggaaagatgtgccgaccagaggcctgagtctag
tctcctgaatctgatctcctacagagctcaatccatccatggtggaagcggcggcagtggaggctccaccctgcccgccagccacgc
caccagagtgtacgagatgctggtcagtcacatccagctgcactacaaacattcctacactctgccaatcgccagctcaatccggctgc
aggccttcgactttctgctgctgctgagagccgacagcctgcacagactcggcctgcctaacaaggatggtgtggtgaggttcagtcc
atactgtgtgtgcgggtctggctctctgctgttcagagtcctgctgcagtgcctcaagcaggagagcgactggaaggtcctgaagctg
gtcctgggccgtctgcccgagagtctgaggtacaaggtgctgattttcaccagcccctgcagtgtggaccagctgtgttccgccctctg
cagcatgctgagcggcccaaagaccctggagcggctgaggggcgcccccgagggatttagcaggaccgatctgcacctggccgt
ggtgcccgtgctgactgccctgatctcctaccacaattacctggataagaccaaacagcgggagatggtgtattgcctggagcagggg
ctgatccatcgctgtgcctctcagtgcgtggtggccctgagtatctgcagcgtggaaatgcccgatatcattatcaaggccctgcctgtg
ctggtggtgaagctgacccacatcagcgccaccgccagcatggccgtgcctctcctggagttcctgagtacactggccagactgccc
catctgtatagaaattttgccgcagaacagtacgccagcgtgttcgccatctctctgccatacacaaatccctccaagttcaaccagtata
tcgtgtgtctcgcccaccacgtgatcgcaatgtggttcatccgctgccggcttcccttcaggaaggacttcgtgccctttatcactaaggg
actgaggagtaacgtgctgctcagtggcagcgggaagaacctgcatctcgagctgaccgagacatgcctggacatgatggctaggt
atgtgttctccaacttcacagccgtgcccaaacggtccccagtgggggaatttctgctggcagggggcagaaccaagacatggctggt
ggggaataagctggtgaccgtgaccacctccgtgggcactggcacccggagcctgctgggcgggagcggcggcctggccgccta
cgtgcctctgctgacacagggctgggcagagatcctggtgagaagacctacaggcaacaccagctggctgatgtccctggagaacc
ccggtagtggcattaatccatcttttgtgtttctgcagctgtaccatagccccttcttcggcgatgaaagcaataagcctatcctgctgccc
aacgagagccagtcattcgagaggagcgtgcagctgctcgatcagatccctagctatgacactcacaaaatcgccgtgctgtacgtgg
gcgaggggcagtccaactctgagctggccatcctgagcaatgagcacgggagctacagatataccgagtttctgaccggcctgggc
cggctgattgagctgaaggattgccagcccgacaaagtgtacctgggggcctggatgtgtgcggggaggacggccagtttacttac
tgttggcacgacgatatcatgcaggccgtgttccacatcgctactctgatgcctacaaaagatgtggacaagcaccggtgcgacaaaa
aaagacatctgggaaatgactttgtgtccattgtgtacaatgactccggcgaggactttaaactgggaaccatcaagggccagttcaatt
tcgtgcacgtgatcgtgactccactggattacgagtgtaatctcgtgagcctgcagtgcaggaaagacatggaagggctggtggatac
ctctgtggccaaaatcgtgtcagacaggaacctgccctttgtggctcggcagatggccctgcacgccaatatggccagtcaggtgcac
cactcccggagcaaccctacagatatctatcccagcaaatggatcgcccgcctgaggcacattaagcgcctgcggcagagaatctgt
gaagaggccgcctacagcaacccatcatga
SEQ ID NO: 34, DNA, Artificial sequence, Micro-Tuberin 8 native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGGGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGAAATTTGAAGAGCACGCAGTGGAAGCACTCTGGAAGGCGGTCGCG
GATCTGTTGCAGCCGGAGCGGCCGCTGGAGGCCCGGCACGCGGTGCTGGCTCTG
CTGAAGGCCATCGTGCAGGGGCAGGGCGAGCGTTTGGGGGTCCTCAGAGCCCTC
TTCTTTAAGGTCATCAAGGATTACCCTTCCAACGAAGACCTTCACGAAAGGCTGG
AGGTTTTCAAGGCCCTCACAGACAATGGGAGACACATCACCTACTTGGAGGAAG
AGCTGGCTGACTTTGTCCTGCAGTGGATGGATGTTGGCTTGTCCTCGGAATTCCTT
CTGGTGCTGGTGAACTTGGTCAAATTCAATAGCTGTTACCTCGACGAGTACATCG
CAAGGATGGTTCAGATGATCTGTCTGCTGTGCGTCCGGACCGCGTCCTCTGTGGA
CATAGAGGTCTCCCTGCAGGTGCTGGACGCCGTGGTCTGCTACAACTGCCTGCCG
GCTGAGAGCCTCCCGCTGTTCATCGTTACCCTCTGTCGCACCATCAACGTCAAGG
AGCTCTGCGAGCCTTGCTGGAAGCTGATGCGGAACCTCCTTGGCACCCACCTGGG
CCACAGCGCCATCTACAACATGTGCCACCTCATGGAGGACAGAGCCTACATGGA
GGACGCGCCCCTGCTGAGAGGAGCCGTGTTTTTTGTGGGCATGGCTCTCTGGGGA
GCCCACCGGCTCTATTCTCTCAGGAACTCGCCGACATCTGTGTTGCCATCATTTTA
CCAGGCCATGGCATGTCCGAACGAGGTGGTGTCCTATGAGATCGTCCTGTCCATC
ACCAGGCTCATCAAGAAGTATAGGAAGGAGCTCCAGGTGGTGGCGTGGGACATT
CTGCTGAACATCATCGAACGGCTCCTTCAGCAGCTCCAGACCTTGGACAGCCCGG
AGCTCAGGACCATCGTCCATGACCTGTTGACCACGGTGGAGGAGCTGTGTGACC
AGAACGAGTTCCACGGGTCTCAGGAGAGATACTTTGAACTGGTGGAGAGATGTG
CGGACCAGAGGCCTGAGTCCTCCCTCCTGAACCTGATCTCCTATAGAGCGCAGTC
CATCCACGGTGGAAGCGGCGGCAGTGGAGGCTCCACCCTGCCTGCAAGCCAC
GCCACGCGTGTGTATGAGATGCTGGTCAGCCACATTCAGCTCCACTACAAGCACA
GCTACACCCTGCCAATCGCGAGCAGCATCCGGCTGCAGGCCTTTGACTTCCTGTT
GCTGCTGCGGGCCGACTCACTGCACCGCCTGGGCCTGCCCAACAAGGATGGAGT
CGTGCGGTTCAGCCCCTACTGCGTCTGCGGGTCTGGCTCCCTGCTCTTCCGCGTC
CTGCTGCAGTGCTTGAAGCAGGAGTCTGACTGGAAGGTGCTGAAGCTGGTTCTG
GGCAGGCTGCCTGAGTCCCTGCGCTATAAAGTGCTCATCTTTACTTCCCCTTGCA
GTGTGGACCAGCTGTGCTCTGCTCTCTGCTCCATGCTTTCAGGCCCAAAGACACT
GGAGCGGCTCCGAGGCGCCCCAGAAGGCTTCTCCAGAACTGACTTGCACCTGGC
CGTGGTTCCAGTGCTGACAGCATTAATCTCTTACCATAACTACCTGGACAAAACC
AAACAGCGCGAGATGGTCTACTGCCTGGAGCAGGGCCTCATCCACCGCTGTGCC
AGCCAGTGCGTCGTGGCCTTGTCCATCTGCAGCGTGGAGATGCCTGACATCATCA
TCAAGGCGCTGCCTGTTCTGGTGGTGAAGCTCACGCACATCTCAGCCACAGCCAG
CATGGCCGTCCCACTGCTGGAGTTCCTGTCCACTCTGGCCAGGCTGCCGCACCTC
TACAGGAACTTTGCCGCGGAGCAGTATGCCAGTGTGTTCGCCATCTCCCTGCCGT
ACACCAACCCCTCCAAGTTTAATCAGTACATCGTGTGTCTGGCCCATCACGTCAT
AGCCATGTGGTTCATCAGGTGCCGCCTGCCCTTCCGGAAGGATTTTGTCCCTTTC
ATCACTAAGGGCCTGCGGTCCAATGTCCTCTTGTCTGGCAGCGGGAAAAACCTC
CACCTGGAGCTCACGGAAACCTGTCTGGACATGATGGCTCGATACGTCTTCTCCA
ACTTCACGGCTGTCCCGAAGAGGTCTCCTGTGGGCGAGTTCCTCCTAGCGGGTGG
CAGGACCAAAACCTGGCTGGTTGGGAACAAGCTTGTCACTGTGACGACAAGCGT
GGGAACCGGGACCCGGTCGTTACTAGGCGGGAGCGGCGGCCTGGCGGCCTATG
TGCCCCTGCTGACCCAGGGCTGGGCGGAGATCCTGGTCCGGAGGCCCACAGGGA
ACACCAGCTGGCTGATGAGCCTGGAGAACCCGGGTAGTGGCATCAACCCCAGTT
TCGTGTTCCTGCAGCTCTACCATTCCCCCTTCTTTGGCGACGAGTCAAACAAGCC
AATCCTGCTGCCCAATGAGTCACAGTCCTTTGAGCGGTCGGTGCAGCTCCTCGAC
CAGATCCCATCATACGACACCCACAAGATCGCCGTCCTGTATGTTGGAGAAGGC
CAGAGCAACAGCGAGCTCGCCATCCTGTCCAATGAGCATGGCTCCTACAGGTAC
ACGGAGTTCCTGACGGGCCTGGGCCGGCTCATCGAGCTGAAGGACTGCCAGCCG
GACAAGGTGTACCTGGGAGGCCTGGACGTGTGTGGTGAGGACGGCCAGTTCACC
TACTGCTGGCACGATGACATCATGCAAGCCGTCTTCCACATCGCCACCCTGATGC
CCACCAAGGACGTGGACAAGCACCGCTGCGACAAGAAGCGCCACCTGGGCAAC
GACTTTGTGTCCATTGTCTACAATGACTCCGGTGAGGACTTCAAGCTTGGCACCA
TCAAGGGCCAGTTCAACTTTGTCCACGTGATCGTCACCCCGCTGGACTACGAGTG
CAACCTGGTGTCCCTGCAGTGCAGGAAAGACATGGAGGGCCTTGTGGACACCAG
CGTGGCCAAGATCGTGTCTGACCGCAACCTGCCCTTCGTGGCCCGCCAGATGGCC
CTGCACGCAAATATGGCCTCACAGGTGCATCATAGCCGCTCCAACCCCACCGATA
TCTACCCCTCCAAGTGGATTGCCCGGCTCCGCCACATCAAGCGGCTCCGCCAGCG
GATCTGCGAGGAAGCCGCCTACTCCAACCCCAGCTGAGGCCGGGGCCCTCCCTC
CTGCACTGGCCTTGGACGGTATTGCCTGTCAGTGAAATAAATAAAGTCCTGACCC
CAGTGCACAGACATAGAGGCACAGATTGCAGTCAGACAGCTCTTTTATTGACTTT
GTCTGCTTGGTGCGGGGGTTGGGGGGGTGTCGAGGCTCTAGAAGCGGCCATGCC
CACAGAAGTGGTACACAGAAGCAGGCACAGCCAGCTCCGAGGGCacccagctttcttgtac
aaagtgggaattcctagagctcgctgatcagcctcgactgtgccttctagttgccagccatctgttgtttgcccctcccccgtgccttc
cttgaccctggaaggtgccactcccactgtcctttcctaataaaatgaggaaattgcatcgcattgtctgagtaggtgtcattcta
ttctggggggtggggtggggcaggacagcaagggggaggattgggaagagaatagcaggcatgctggggagggccgcag
gaacccctagtgatggagttggccactccctctctgcgcgctcgctcgctcactgaggccgggcgaccaaaggtcgcccgacgccc
gggctttgcccgggcggcctcagtgagcgagcgagcgcgcag
SEQ ID NO: 35, DNA, Artificial sequence, Micro-Tuberin 8 codon-optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTC
CATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCC
CGCCTGGCATTATGCCCAGTACATGACCTTATGGGACTTTCCTACTTGGCAGTAC
ATCTACTCGAGGCCACGTTCTGCTTCACTCTCCCCATCTCCCCCCCCTCCCCACCC
CCAATTTTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGG
CGGGCGGGAGCGGGATCAGCCACCGCGGTGGCGGCCTAGAGTCGACGAGGAAC
TGAAAAACCAGAAAGTTAACTGGTAAGTTTAGTCTTTTTGTCTTTTATTTCAGGTC
CCGGATCCGGTGGTGGTGCAAATCAAAGAACTGCTCCTCAGTGGATGTTGCCTTT
ACTTCTAGGCCTGTACGGAAGTGTTACTTCTGCTCTAAAAGCTGCGGAATTGTAC
CCGCGGCCGATCCACCGGTCCGGAATTCCCGGGATATCGTCGACCCACGCGTCCG
GGCCCCACGCTGCGCACCCGCGGGTTTGCTCAAGTTTGTACAAAAAAGCAGGCT
GCCACCATGAAGTTTGAAGAGCATGCCGTGGAAGCCCTGTGGAAGGCCGTGGCT
GACCTGCTGCAGCCTGAACGTCCTCTGGAAGCTCGCCATGCCGTGTTGGCCCTGC
TGAAGGCTATCGTGCAGGGACAGGGAGAGAGGCTGGGCGTGCTGCGCGCCCTGT
TCTTTAAGGTGATTAAGGACTACCCCTCCAACGAGGACCTGCACGAGAGGCTGG
AAGTGTTTAAGGCCCTGACCGATAACGGCAGGCACATCACTTACCTGGAGGAGG
AGCTGGCCGACTTCGTGCTGCAGTGGATGGATGTGGGCCTGAGCAGCGAATTCCT
GCTGGTGCTGGTGAATCTGGTGAAATTCAACAGCTGTTACCTGGATGAATACATT
GCCAGAATGGTCCAGATGATCTGCCTGCTTTGCGTGCGAACAGCCTCATCAGTGG
ATATCGAGGTGTCTCTGCAGGTGCTGGACGCCGTGGTGTGCTATAACTGTCTGCC
CGCTGAGAGCCTCCCTCTGTTTATCGTGACACTGTGCCGCACCATCAACGTGAAA
GAACTGTGCGAGCCTTGCTGGAAACTGATGAGAAACCTGCTGGGGACACACCTG
GGCCACAGCGCCATCTACAACATGTGCCACCTCATGGAAGACCGGGCCTACATG
GAGGATGCCCCCCTGCTGCGCGGGGCCGTGTTTTTCGTGGGAATGGCTCTGTGGG
GGGCCCATAGACTGTACAGCCTCAGGAATTCCCCAACCAGCGTGCTGCCATCCTT
TTATCAGGCCATGGCCTGTCCCAACGAAGTGGTGAGCTATGAGATCGTCCTGTCC
ATCACCCGGCTGATTAAAAAATACCGGAAAGAGCTGCAGGTGGTGGCTTGGGAT
ATCCTGCTGAATATCATCGAGAGGCTGCTGCAGCAGCTGCAGACTCTCGACAGCC
CTGAGCTGCGCACCATTGTGCACGATTTGCTGACCACCGTGGAAGAGCTGTGCGA
TCAGAACGAATTCCACGGCTCCCAGGAGAGGTACTTTGAGCTGGTGGAAAGATG
TGCCGACCAGAGGCCTGAGTCTAGTCTCCTGAATCTGATCTCCTACAGAGCTCAA
TCCATCCATGGTGGAAGCGGCGGCAGTGGAGGCTCCACCCTGCCCGCCAGCC
ACGCCACCAGAGTGTACGAGATGCTGGTCAGTCACATCCAGCTGCACTACAAAC
ATTCCTACACTCTGCCAATCGCCAGCTCAATCCGGCTGCAGGCCTTCGACTTTCT
GCTGCTGCTGAGAGCCGACAGCCTGCACAGACTCGGCCTGCCTAACAAGGATGG
TGTGGTGAGGTTCAGTCCATACTGTGTGTGCGGGTCTGGCTCTCTGCTGTTCAGA
GTCCTGCTGCAGTGCCTCAAGCAGGAGAGCGACTGGAAGGTCCTGAAGCTGGTC
CTGGGCCGTCTGCCCGAGAGTCTGAGGTACAAGGTGCTGATTTTCACCAGCCCCT
GCAGTGTGGACCAGCTGTGTTCCGCCCTCTGCAGCATGCTGAGCGGCCCAAAGA
CCCTGGAGCGGCTGAGGGGCGCCCCCGAGGGATTTAGCAGGACCGATCTGCACC
TGGCCGTGGTGCCCGTGCTGACTGCCCTGATCTCCTACCACAATTACCTGGATAA
GACCAAACAGCGGGAGATGGTGTATTGCCTGGAGCAGGGGCTGATCCATCGCTG
TGCCTCTCAGTGCGTGGTGGCCCTGAGTATCTGCAGCGTGGAAATGCCCGATATC
ATTATCAAGGCCCTGCCTGTGCTGGTGGTGAAGCTGACCCACATCAGCGCCACCG
CCAGCATGGCCGTGCCTCTCCTGGAGTTCCTGAGTACACTGGCCAGACTGCCCCA
TCTGTATAGAAATTTTGCCGCAGAACAGTACGCCAGCGTGTTCGCCATCTCTCTG
CCATACACAAATCCCTCCAAGTTCAACCAGTATATCGTGTGTCTCGCCCACCACG
TGATCGCAATGTGGTTCATCCGCTGCCGGCTTCCCTTCAGGAAGGACTTCGTGCC
CTTTATCACTAAGGGACTGAGGAGTAACGTGCTGCTCAGTGGCAGCGGGAAGA
ACCTGCATCTCGAGCTGACCGAGACATGCCTGGACATGATGGCTAGGTATGTGTT
CTCCAACTTCACAGCCGTGCCCAAACGGTCCCCAGTGGGGGAATTTCTGCTGGCA
GGGGGCAGAACCAAGACATGGCTGGTGGGGAATAAGCTGGTGACCGTGACCACC
TCCGTGGGCACTGGCACCCGGAGCCTGCTGGGCGGGAGCGGCGGCCTGGCCGC
CTACGTGCCTCTGCTGACACAGGGCTGGGCAGAGATCCTGGTGAGAAGACCTAC
AGGCAACACCAGCTGGCTGATGTCCCTGGAGAACCCCGGTAGTGGCATTAATCC
ATCTTTTGTGTTTCTGCAGCTGTACCATAGCCCCTTCTTCGGCGATGAAAGCAATA
AGCCTATCCTGCTGCCCAACGAGAGCCAGTCATTCGAGAGGAGCGTGCAGCTGC
TCGATCAGATCCCTAGCTATGACACTCACAAAATCGCCGTGCTGTACGTGGGCGA
GGGGCAGTCCAACTCTGAGCTGGCCATCCTGAGCAATGAGCACGGGAGCTACAG
ATATACCGAGTTTCTGACCGGCCTGGGCCGGCTGATTGAGCTGAAGGATTGCCAG
CCCGACAAAGTGTACCTGGGCGGCCTGGATGTGTGCGGGGAGGACGGCCAGTTT
ACTTACTGTTGGCACGACGATATCATGCAGGCCGTGTTCCACATCGCTACTCTGA
TGCCTACAAAAGATGTGGACAAGCACCGGTGCGACAAAAAAAGACATCTGGGA
AATGACTTTGTGTCCATTGTGTACAATGACTCCGGCGAGGACTTTAAACTGGGAA
CCATCAAGGGCCAGTTCAATTTCGTGCACGTGATCGTGACTCCACTGGATTACGA
GTGTAATCTCGTGAGCCTGCAGTGCAGGAAAGACATGGAAGGGCTGGTGGATAC
CTCTGTGGCCAAAATCGTGTCAGACAGGAACCTGCCCTTTGTGGCTCGGCAGATG
GCCCTGCACGCCAATATGGCCAGTCAGGTGCACCACTCCCGGAGCAACCCTACA
GATATCTATCCCAGCAAATGGATCGCCCGCCTGAGGCACATTAAGCGCCTGCGG
CAGAGAATCTGTGAAGAGGCCGCCTACAGCAACCCATCATGAacccagctttcttgtacaaag
tgggaattcctagagctcgctgatcagcctcgactgtgccttctagttgccagccatctgttgtttgcccctcccccgtgccttccttg
accctggaaggtgccactcccactgtcctttcctaataaaatgaggaaattgcatcgcattgtctgagtaggtgtcattctattct
ggggggtggggtggggcaggacagcaagggggaggattgggaagagaatagcaggcatgctggggagggccgcaggaac
ccctagtgatggagttggccactccctctctgcgcgctcgctcgctcactgaggccgggcgaccaaaggtcgcccgacgcccgggc
tttgcccgggcggcctcagtgagcgagcgagcgcgcag
SEQ ID NO: 36, Protein, Artificial sequence, Micro-Tuberin 8 protein sequence
MKFEEHAVEALWKAVADLLQPERPLEARHAVLALLKAIVQGQGERLGVLRALFFK
VIKDYPSNEDLHERLEVFKALTDNGRHITYLEEELADFVLQWMDVGLSSEFLLVLVN
LVKFNSCYLDEYIARMVQMICLLCVRTASSVDIEVSLQVLDAVVCYNCLPAESLPLFI
VTLCRTINVKELCEPCWKLMRNLLGTHLGHSAIYNMCHLMEDRAYMEDAPLLRGA
VFFVGMALWGAHRLYSLRNSPTSVLPSFYQAMACPNEVVSYEIVLSITRLIKKYRKE
LQVVAWDILLNIIERLLQQLQTLDSPELRTIVHDLLTTVEELCDQNEFHGSQERYFEL
VERCADQRPESSLLNLISYRAQSIHGGSGGSGGSTLPASHATRVYEMLVSHIQLHYK
HSYTLPIASSIRLQAFDFLLLLRADSLHRLGLPNKDGVVRFSPYCVCGSGSLLFRVLL
QCLKQESDWKVLKLVLGRLPESLRYKVLIFTSPCSVDQLCSALCSMLSGPKTLERLR
GAPEGFSRTDLHLAVVPVLTALISYHNYLDKTKQREMVYCLEQGLIHRCASQCVVA
LSICSVEMPDIIIKALPVLVVKLTHISATASMAVPLLEFLSTLARLPHLYRNFAAEQYA
SVFAISLPYTNPSKFNQYIVCLAHHVIAMWFIRCRLPFRKDFVPFITKGLRSNVLLSGS
GKNLHLELTETCLDMMARYVFSNFTAVPKRSPVGEFLLAGGRTKTWLVGNKLVTV
TTSVGTGTRSLLGGSGGLAAYVPLLTQGWAEILVRRPTGNTSWLMSLENPGSGINPS
FVFLQLYHSPFFGDESNKPILLPNESQSFERSVQLLDQIPSYDTHKIAVLYVGEGQSNS
ELAILSNEHGSYRYTEFLTGLGRLIELKDCQPDKVYLGGLDVCGEDGQFTYCWHDDI
MQAVFHIATLMPTKDVDKHRCDKKRHLGNDFVSIVYNDSGEDFKLGTIKGQFNFVH
VIVTPLDYECNLVSLQCRKDMEGLVDTSVAKIVSDRNLPFVARQMALHANMASQV
HHSRSNPTDIYPSKWIARLRHIKRLRQRICEEAAYSNPS
SEQ ID NO: 37, DNA, Artificial sequence, Micro-Tuberin 9 coding sequence
atggccaaaccaacaagcaaagattcaggcttgaaggagaagtttaagattctgttgggactgggaacaccgaggccaaatcccagg
tctgcagagggtaaacagacggagtttatcatcaccgcggaaatactgagagaactgagcatggaatgtggcctcaacaatcgcatcc
ggatgatagggcagatttgtgaagtcgcaaaaaccaagaaatttgaagagcacgcagtggaagcactctggaaggcggtcgcggat
ctgttgcagccggagcggccgctggaggcccggcacgcggtgctggctctgctgaaggccatcgtgcaggggcagggcgagcgt
ttgggggtcctcagagccctcttctttaaggtcatcaaggattacccttccaacgaagaccttcacgaaaggctggaggttttcaaggcc
ctcacagacaatgggagacacatcacctacttggaggaagagctggctgactttgtcctgcagtggatggatgttggcttgtcctcgga
attccttctggtgctggtgaacttggtcaaattcaatagctgttacctcgacgagtacatcgcaaggatggttcagatgatctgtctgctgt
gcgtccggaccgcgtcctctgtggacatagaggtctccctgcaggtgctggacgccgtggtctgctacaactgcctgccggctgaga
gcctcccgctgttcatcgttaccctctgtcgcaccatcaacgtcaaggagctctgcgagccttgctggaagctgatgcggaacctccttg
gcacccacctgggccacagcgccatctacaacatgtgccacctcatggaggacagagcctacatggaggacgcgcccctgctgag
aggagccgtgttttttgtgggcatggctctctggggagcccaccggctctattctctcaggaactcgccgacatctgtgttgccatcatttt
accaggccatggcatgtccgaacgaggtggtgtcctatgagatcgtcctgtccatcaccaggctcatcaagaagtataggaaggagct
ccaggtggtggcgtgggacattctgctgaacatcatcgaacggctccttcagcagctccagaccttggacagcccggagctcaggac
catcgtccatgacctgttgaccacggtggaggagctgtgtgaccagaacgagttccacgggtctcaggagagatactttgaactggtg
gagagatgtgcggaccagaggcctgagtcctccctcctgaacctgatctcctatagagcgcagtccatccacccggccaaggacgg
ctggattcagaacctgcaggcgctgatggagagattcttcaggagcgagtcccgaggcgccgtgcgcatcaaggtgctggacgtgct
gtcctttgtgctgctcatcaacaggcagttctatgaggaggagctgattaactcagtggtcatctcgcagctctcccacatccccgagga
taaagaccaccaggtccgaaagctggccacccagttgctggtggacctggcagagggctgccacacacaccacttcaacagcctgc
tggacatcatcgagaaggtgatggcccgctccctctccccacccccggagctggaagaaagggatgtggccgcatactcggcctcct
tggaggatgtgaagacagccgtcctggggcttctggtcatccttcagaccaagctgtacaccctgcctgcaagccacgccacgcgtgt
gtatgagatgctggtcagccacattcagctccactacaagcacagctacaccctgccaatcgcgagcagcatccggctgcaggccttt
gacttcctgttgctgctgcgggccgactcactgcaccgcctgggcctgcccaacaaggatggagtcgtgcggttcagcccctactgcg
tctgcggcagcggctccctgctcttccgcgtcctgctgcagtgcttgaagcaggagtctgactggaaggtgctgaagctggttctggg
caggctgcctgagtccctgcgctataaagtgctcatctttacttccccttgcagtgtggaccagctgtgctctgctctctgctccatgctttc
aggcccaaagacactggagcggctccgaggcgccccagaaggcttctccagaactgacttgcacctggccgtggttccagtgctga
cagcattaatctcttaccataactacctggacaaaaccaaacagcgcgagatggtctactgcctggagcagggcctcatccaccgctgt
gccagccagtgcgtcgtggccttgtccatctgcagcgtggagatgcctgacatcatcatcaaggcgctgcctgttctggtggtgaagct
cacgcacatctcagccacagccagcatggccgtcccactgctggagttcctgtccactctggccaggctgccgcacctctacaggaa
ctttgccgcggagcagtatgccagtgtgttcgccatctccctgccgtacaccaacccctccaagtttaatcagtacatcgtgtgtctggcc
catcacgtcatagccatgtggttcatcaggtgccgcctgcccttccggaaggattttgtccctttcatcactaagggcctgcggtccaatg
tcctcttgtctgggagcggcaaaaacctccacctggagctcacggaaacctgtctggacatgatggctcgatacgtcttctccaacttca
cggctgtcccgaagaggtctcctgtgggcgagttcctcctagcgggtggcaggaccaaaacctggctggttgggaacaagcttgtca
ctgtgacgacaagcgtgggaaccgggacccggtcgttactaggcggcagcggaggcctggcggcctatgtgcccctgctgaccca
gggctgggcggagatcctggtccggaggcccacagggaacaccagctggctgatgagcctggagaacccgggaagtggtatcaa
ccccagtttcgtgttcctgcagctctaccattcccccttctttggcgacgagtcaaacaagccaatcctgctgcccaatgagtcacagtcc
tttgagcggtcggtgcagctcctcgaccagatcccatcatacgacacccacaagatcgccgtcctgtatgttggagaaggccagagca
acagcgagctcgccatcctgtccaatgagcatggctcctacaggtacacggagttcctgacgggcctgggccggctcatcgagctga
aggactgccagccggacaaggtgtacctgggaggcctggacgtgtgtggtgaggacggccagttcacctactgctggcacgatgac
atcatgcaagccgtcttccacatcgccaccctgatgcccaccaaggacgtggacaagcaccgctgcgacaagaagcgccacctggg
caacgactttgtgtccattgtctacaatgactccggtgaggacttcaagcttggcaccatcaagggccagttcaactttgtccacgtgatc
gtcaccccgctggactacgagtgcaacctggtgtccctgcagtgcaggaaagacatggagggccttgtggacaccagcgtggccaa
gatcgtgtctgaccgcaacctgcccttcgtggcccgccagatggccctgcacgcaaatatggcctcacaggtgcatcatagccgctcc
aaccccaccgatatctacccctccaagtggattgcccggctccgccacatcaagcggctccgccagcggatctgcgaggaagccgc
ctactccaaccccagctga
SEQ ID NO: 38, DNA, Artificial sequence, Micro-Tuberin 9 codon-optimized
sequence
atggctaagccaacttctaaggattctggcctgaaggagaagttcaagatcctgctggggctcggcactccacggcctaatcctagatc
tgccgagggaaagcagaccgagtttatcatcaccgccgaaatcctgagagagctgagcatggaatgcgggctcaataacaggatcc
ggatgatcggccagatttgcgaggtggctaagaccaagaaatttgaagagcatgccgtggaggccctctggaaagccgtggccgac
ctgttacagcctgagagacccttggaggcccggcacgcagtgctggctctgctgaaagcaatcgtgcagggacagggggagaggc
tgggcgtgctgagagccctgttcttcaaggtgatcaaggattacccaagcaatgaggatctgcatgaaagactggaggtgtttaaggcc
ctgactgacaacgggcgacacatcacatatctggaggaggagctggccgacttcgtgctgcagtggatggatgtgggcctgtcctcc
gagttcctgctggtgctggtcaatctggtgaagttcaatagctgctatctggacgaatacatcgcccggatggtgcagatgatctgcctg
ctgtgtgtgaggacagccagcagcgtggacatcgaggtgtcactccaggtgctggatgccgtggtgtgttacaactgtctccccgccg
agagcctgcccctgtttatcgtcaccctgtgcagaaccattaacgtgaaggaactgtgtgagccatgttggaagctgatgcggaacctg
ctgggcacccacctgggccattctgccatctacaatatgtgccacctgatggaggatcgggcctatatggaggacgcccctctgctga
gaggcgccgtgtttttcgtcggcatggcactctggggcgcccaccgactgtatagcctccgcaactcccctaccagcgtgctgccctct
ttctatcaggccatggcctgccccaatgaggtcgtgtcttacgagatcgtgctcagcatcaccaggctgattaagaagtacaggaagga
gctgcaggtggtggcctgggatattctgttaaacatcatcgagcgcctgctgcagcagctgcagactctggactcccctgagctgcgg
accattgtccacgatctgctgaccaccgtggaggagctgtgcgatcagaacgaattccacggctctcaggagcggtattttgagctggt
ggagcggtgcgccgaccagagacctgagtcatcactgctgaacctgatctcctacagggctcagtccatccacccagccaaagacg
gctggatccagaacctgcaggcactgatggagcgcttcttccggtctgaatctcggggcgccgtaaggatcaaggtgctggatgtgct
gagcttcgtgctgctgatcaaccggcagttctacgaggaggagctgatcaactctgtggtgatcagtcagctgtctcatatccccgagg
ataaagaccaccaggtgagaaagctggccacccagctgctcgtggatctggccgagggctgccacacacaccacttcaacagcctg
ctggacatcatcgaaaaagtgatggcccgttccctgagccccccacccgagctggaagagcgcgatgtggccgcttattctgccagc
ctggaagacgtgaaaactgccgtcctgggcctgctggtgattctgcagaccaagctgtatacattgccagccagtcacgccaccagg
gtgtacgagatgctggtgagccatatccagctccactataagcactcttataccctgccaattgccagctccattaggctgcaggcctttg
acttcctgctgctgctgagggctgacagcctgcacagactgggactgcctaacaaggatggggggtgcgcttcagcccctactgcgt
gtgcggcagcggctctctgctgtttagggtgctgctgcagtgtctcaaacaggagagcgactggaaagtgctgaaactggtgctggg
ccgcctgcccgaatccctgaggtacaaagtgctgatcttcacatccccatgctccgtggaccagctgtgttcagccctgtgctcaatgct
gtccggacccaaaacactggagagactgaggggcgccccagagggcttttccaggacagacctgcacctggcagtggtgcctgtgt
tgaccgcactgatctcctatcacaactatctggataagaccaagcagagggagatggtgtactgcctggagcagggactgattcaccg
ttgcgccagccagtgcgtggtcgctctgtcaatctgcagtgtggagatgcccgatatcatcatcaaggctctgcctgtcctggtggtgaa
gctgacacacatcagcgccaccgccagcatggctgtgcccctgctggagtttctgagcactctggcccggctgccccacctgtaccgt
aactttgccgccgagcagtacgcctccgtgtttgccattagcttgccctacaccaacccatccaagttcaatcaatatatcgtgtgcctgg
cccaccacgtgatcgctatgtggttcatcagatgccgcctccccttccggaaggatttcgtgccctttatcaccaaagggctgaggtcta
acgtgctgctctctgggagcggcaagaatctgcacctggaactgaccgagacctgcctggacatgatggccaggtacgtgttttccaa
ctttacagccgtgcctaaacgctccccagtcggcgaatttctgctggccgggggccgcactaagacctggctcgtgggcaacaagct
cgtgaccgtgaccaccagcgtgggtactgggacacggtccctgctgggcggcagcggaggcctggccgcctacgtgcctctgctta
cacagggctgggccgaaatcctggtgcgacgcccaacgggaaacacctcttggctgatgtccctggagaatcctggaagtggtatc
aatcctagcttcgtgtttctgcagctgtaccacagcccttttttcggcgacgagtccaataaacccatcctgctgcctaacgagtcccaga
gcttcgagcggtcagtgcagcttctggatcagatcccaagctacgacacacacaagatcgctgtgctgtatgttggcgaaggccagag
taatagcgagctggccatcctgagcaatgagcacggcagctaccgctatacagagttcctgaccggcctcggaaggctgattgagct
gaaggattgtcagccagataaggtctatctgggcggcctggatgtgtgcggcgaggatggccagtttacatattgctggcacgatgata
tcatgcaggccgtgttccacatcgccacactgatgcctaccaaagatgtggacaagcacaggtgcgacaagaaaagacacctgggc
aacgacttcgtgtccatcgtgtacaatgacagtggagaggacttcaagctgggcaccattaagggccagttcaatttcgtgcacgttatt
gttacacctctggattacgaatgcaacctcgtgagtctgcagtgtaggaaggacatggagggcttggtggacacctctgtggccaagat
tgtgagcgacagaaatctgccctttgtggccaggcagatggcactgcatgccaatatggcctctcaggtgcaccactcccggtcaaat
cccactgacatctatccctccaagtggatcgccagactgagacatattaagcgcctgcgccagcgaatctgtgaggaggccgcctatt
ccaacccatcatga
SEQ ID NO: 39, DNA, Artificial sequence, Micro-Tuberin 9 native construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GGGGCAGAGCGCACATCGCCCACAGTCCCCGAGAAGTTGGGGGGAGGGGTCGG
CAATTGAACCGGTGCCTAGAGAAGGTGGCGCGGGGTAAACTGGGAAAGTGATGT
CGTGTACTGGCTCCGCCTTTTTCCCGAGGGTGGGGGAGAACCGTATATAAGTGCA
GTAGTCGCCGTGAACGTTCTTTTTCGCAACGGGTTTGCCGCCAGAACACAGGTAA
GTTTAGTCTTTTTGTCTTTTATTTCAGGTCCCGGATCCGGTGGTGGTGCAAATCAA
AGAACTGCTCCTCAGTGGATGTTGCCTTTACTTCTAGCAAGTTTGTACAAAAAAG
CAGGCTGCCACCATGGCCAAACCAACAAGCAAAGATTCAGGCTTGAAGGAGAAG
TTTAAGATTCTGTTGGGACTGGGAACACCGAGGCCAAATCCCAGGTCTGCAGAG
GGTAAACAGACGGAGTTTATCATCACCGCGGAAATACTGAGAGAACTGAGCATG
GAATGTGGCCTCAACAATCGCATCCGGATGATAGGGCAGATTTGTGAAGTCGCA
AAAACCAAGAAATTTGAAGAGCACGCAGTGGAAGCACTCTGGAAGGCGGTCGC
GGATCTGTTGCAGCCGGAGCGGCCGCTGGAGGCCCGGCACGCGGTGCTGGCTCT
GCTGAAGGCCATCGTGCAGGGGCAGGGCGAGCGTTTGGGGGTCCTCAGAGCCCT
CTTCTTTAAGGTCATCAAGGATTACCCTTCCAACGAAGACCTTCACGAAAGGCTG
GAGGTTTTCAAGGCCCTCACAGACAATGGGAGACACATCACCTACTTGGAGGAA
GAGCTGGCTGACTTTGTCCTGCAGTGGATGGATGTTGGCTTGTCCTCGGAATTCC
TTCTGGTGCTGGTGAACTTGGTCAAATTCAATAGCTGTTACCTCGACGAGTACAT
CGCAAGGATGGTTCAGATGATCTGTCTGCTGTGCGTCCGGACCGCGTCCTCTGTG
GACATAGAGGTCTCCCTGCAGGTGCTGGACGCCGTGGTCTGCTACAACTGCCTGC
CGGCTGAGAGCCTCCCGCTGTTCATCGTTACCCTCTGTCGCACCATCAACGTCAA
GGAGCTCTGCGAGCCTTGCTGGAAGCTGATGCGGAACCTCCTTGGCACCCACCTG
GGCCACAGCGCCATCTACAACATGTGCCACCTCATGGAGGACAGAGCCTACATG
GAGGACGCGCCCCTGCTGAGAGGAGCCGTGTTTTTTGTGGGCATGGCTCTCTGGG
GAGCCCACCGGCTCTATTCTCTCAGGAACTCGCCGACATCTGTGTTGCCATCATT
TTACCAGGCCATGGCATGTCCGAACGAGGTGGTGTCCTATGAGATCGTCCTGTCC
ATCACCAGGCTCATCAAGAAGTATAGGAAGGAGCTCCAGGTGGTGGCGTGGGAC
ATTCTGCTGAACATCATCGAACGGCTCCTTCAGCAGCTCCAGACCTTGGACAGCC
CGGAGCTCAGGACCATCGTCCATGACCTGTTGACCACGGTGGAGGAGCTGTGTG
ACCAGAACGAGTTCCACGGGTCTCAGGAGAGATACTTTGAACTGGTGGAGAGAT
GTGCGGACCAGAGGCCTGAGTCCTCCCTCCTGAACCTGATCTCCTATAGAGCGCA
GTCCATCCACCCGGCCAAGGACGGCTGGATTCAGAACCTGCAGGCGCTGATGGA
GAGATTCTTCAGGAGCGAGTCCCGAGGCGCCGTGCGCATCAAGGTGCTGGACGT
GCTGTCCTTTGTGCTGCTCATCAACAGGCAGTTCTATGAGGAGGAGCTGATTAAC
TCAGTGGTCATCTCGCAGCTCTCCCACATCCCCGAGGATAAAGACCACCAGGTCC
GAAAGCTGGCCACCCAGTTGCTGGTGGACCTGGCAGAGGGCTGCCACACACACC
ACTTCAACAGCCTGCTGGACATCATCGAGAAGGTGATGGCCCGCTCCCTCTCCCC
ACCCCCGGAGCTGGAAGAAAGGGATGTGGCCGCATACTCGGCCTCCTTGGAGGA
TGTGAAGACAGCCGTCCTGGGGCTTCTGGTCATCCTTCAGACCAAGCTGTACACC
CTGCCTGCAAGCCACGCCACGCGTGTGTATGAGATGCTGGTCAGCCACATTCAGC
TCCACTACAAGCACAGCTACACCCTGCCAATCGCGAGCAGCATCCGGCTGCAGG
CCTTTGACTTCCTGTTGCTGCTGCGGGCCGACTCACTGCACCGCCTGGGCCTGCC
CAACAAGGATGGAGTCGTGCGGTTCAGCCCCTACTGCGTCTGCGGCAGCGGCTC
CCTGCTCTTCCGCGTCCTGCTGCAGTGCTTGAAGCAGGAGTCTGACTGGAAGGTG
CTGAAGCTGGTTCTGGGCAGGCTGCCTGAGTCCCTGCGCTATAAAGTGCTCATCT
TTACTTCCCCTTGCAGTGTGGACCAGCTGTGCTCTGCTCTCTGCTCCATGCTTTCA
GGCCCAAAGACACTGGAGCGGCTCCGAGGCGCCCCAGAAGGCTTCTCCAGAACT
GACTTGCACCTGGCCGTGGTTCCAGTGCTGACAGCATTAATCTCTTACCATAACT
ACCTGGACAAAACCAAACAGCGCGAGATGGTCTACTGCCTGGAGCAGGGCCTCA
TCCACCGCTGTGCCAGCCAGTGCGTCGTGGCCTTGTCCATCTGCAGCGTGGAGAT
GCCTGACATCATCATCAAGGCGCTGCCTGTTCTGGTGGTGAAGCTCACGCACATC
TCAGCCACAGCCAGCATGGCCGTCCCACTGCTGGAGTTCCTGTCCACTCTGGCCA
GGCTGCCGCACCTCTACAGGAACTTTGCCGCGGAGCAGTATGCCAGTGTGTTCGC
CATCTCCCTGCCGTACACCAACCCCTCCAAGTTTAATCAGTACATCGTGTGTCTG
GCCCATCACGTCATAGCCATGTGGTTCATCAGGTGCCGCCTGCCCTTCCGGAAGG
ATTTTGTCCCTTTCATCACTAAGGGCCTGCGGTCCAATGTCCTCTTGTCTGGGAG
CGGCAAAAACCTCCACCTGGAGCTCACGGAAACCTGTCTGGACATGATGGCTCG
ATACGTCTTCTCCAACTTCACGGCTGTCCCGAAGAGGTCTCCTGTGGGCGAGTTC
CTCCTAGCGGGTGGCAGGACCAAAACCTGGCTGGTTGGGAACAAGCTTGTCACT
GTGACGACAAGCGTGGGAACCGGGACCCGGTCGTTACTAGGCGGCAGCGGAGG
CCTGGCGGCCTATGTGCCCCTGCTGACCCAGGGCTGGGCGGAGATCCTGGTCCG
GAGGCCCACAGGGAACACCAGCTGGCTGATGAGCCTGGAGAACCCGGGAAGTG
GTATCAACCCCAGTTTCGTGTTCCTGCAGCTCTACCATTCCCCCTTCTTTGGCGAC
GAGTCAAACAAGCCAATCCTGCTGCCCAATGAGTCACAGTCCTTTGAGCGGTCG
GTGCAGCTCCTCGACCAGATCCCATCATACGACACCCACAAGATCGCCGTCCTGT
ATGTTGGAGAAGGCCAGAGCAACAGCGAGCTCGCCATCCTGTCCAATGAGCATG
GCTCCTACAGGTACACGGAGTTCCTGACGGGCCTGGGCCGGCTCATCGAGCTGA
AGGACTGCCAGCCGGACAAGGTGTACCTGGGAGGCCTGGACGTGTGTGGTGAGG
ACGGCCAGTTCACCTACTGCTGGCACGATGACATCATGCAAGCCGTCTTCCACAT
CGCCACCCTGATGCCCACCAAGGACGTGGACAAGCACCGCTGCGACAAGAAGCG
CCACCTGGGCAACGACTTTGTGTCCATTGTCTACAATGACTCCGGTGAGGACTTC
AAGCTTGGCACCATCAAGGGCCAGTTCAACTTTGTCCACGTGATCGTCACCCCGC
TGGACTACGAGTGCAACCTGGTGTCCCTGCAGTGCAGGAAAGACATGGAGGGCC
TTGTGGACACCAGCGTGGCCAAGATCGTGTCTGACCGCAACCTGCCCTTCGTGGC
CCGCCAGATGGCCCTGCACGCAAATATGGCCTCACAGGTGCATCATAGCCGCTCC
AACCCCACCGATATCTACCCCTCCAAGTGGATTGCCCGGCTCCGCCACATCAAGC
GGCTCCGCCAGCGGATCTGCGAGGAAGCCGCCTACTCCAACCCCAGCTGAGGCC
GGGGCCCTCCCTCCTGCACTGGCCTTGGACGGTATTGCCTGTCAGTGAAATAAAT
AAAGTCCTGACCCCAGTGCACAGACATAGAGGCACAGATTGCAGTCAGACAGCT
CTTTTATTGACTTTGTCTGCTTGGTGCGGGGGTTGGGGGGGTGTCGAGGCTCTAG
AAGCGGCCATGCCCACAGAAGTGGTACACAGAAGCAGGCACAGCCAGCTCCGA
GGGCacccagctttcttgtacaaagtgggaattcctagagctcgctgatcagcctcgactgtgccttctagttgccagccatctgtt
gtttgcccctcccccgtgccttccttgaccctggaaggtgccactcccactgtcctttcctaataaaatgaggaaattgcatcgca
ttgtctgagtaggtgtcattctattctggggggtggggtggggcaggacagcaagggggaggattgggaagagaatagcagg
catgctggggagggccgcaggaacccctagtgatggagttggccactccctctctgcgcgctcgctcgctcactgaggccgggcga
ccaaaggtcgcccgacgcccgggctttgcccgggcggcctcagtgagcgagcgagcgcgcag
SEQ ID NO: 40, DNA, Artificial sequence, Micro-Tuberin 9 codon-optimized
construct
ctgcgcgctcgctcgctcactgaggccgcccgggcaaagcccgggcgtcgggcgacctttggtcgcccggcctcagtgagcgagc
gagcgcgcagagagggagtggccaactccatcactaggggttcctTCTAGACAACTTTGTATAGAAAAGTT
GGGGCAGAGCGCACATCGCCCACAGTCCCCGAGAAGTTGGGGGGAGGGGTCGG
CAATTGAACCGGTGCCTAGAGAAGGTGGCGCGGGGTAAACTGGGAAAGTGATGT
CGTGTACTGGCTCCGCCTTTTTCCCGAGGGTGGGGGAGAACCGTATATAAGTGCA
GTAGTCGCCGTGAACGTTCTTTTTCGCAACGGGTTTGCCGCCAGAACACAGGTAA
GTTTAGTCTTTTTGTCTTTTATTTCAGGTCCCGGATCCGGTGGTGGTGCAAATCAA
AGAACTGCTCCTCAGTGGATGTTGCCTTTACTTCTAGCAAGTTTGTACAAAAAAG
CAGGCTGCCACCATGGCTAAGCCAACTTCTAAGGATTCTGGCCTGAAGGAGAAG
TTCAAGATCCTGCTGGGGCTCGGCACTCCACGGCCTAATCCTAGATCTGCCGAGG
GAAAGCAGACCGAGTTTATCATCACCGCCGAAATCCTGAGAGAGCTGAGCATGG
AATGCGGGCTCAATAACAGGATCCGGATGATCGGCCAGATTTGCGAGGTGGCTA
AGACCAAGAAATTTGAAGAGCATGCCGTGGAGGCCCTCTGGAAAGCCGTGGCCG
ACCTGTTACAGCCTGAGAGACCCTTGGAGGCCCGGCACGCAGTGCTGGCTCTGCT
GAAAGCAATCGTGCAGGGACAGGGGGAGAGGCTGGGCGTGCTGAGAGCCCTGTT
CTTCAAGGTGATCAAGGATTACCCAAGCAATGAGGATCTGCATGAAAGACTGGA
GGTGTTTAAGGCCCTGACTGACAACGGGCGACACATCACATATCTGGAGGAGGA
GCTGGCCGACTTCGTGCTGCAGTGGATGGATGTGGGCCTGTCCTCCGAGTTCCTG
CTGGTGCTGGTCAATCTGGTGAAGTTCAATAGCTGCTATCTGGACGAATACATCG
CCCGGATGGTGCAGATGATCTGCCTGCTGTGTGTGAGGACAGCCAGCAGCGTGG
ACATCGAGGTGTCACTCCAGGTGCTGGATGCCGTGGTGTGTTACAACTGTCTCCC
CGCCGAGAGCCTGCCCCTGTTTATCGTCACCCTGTGCAGAACCATTAACGTGAAG
GAACTGTGTGAGCCATGTTGGAAGCTGATGCGGAACCTGCTGGGCACCCACCTG
GGCCATTCTGCCATCTACAATATGTGCCACCTGATGGAGGATCGGGCCTATATGG
AGGACGCCCCTCTGCTGAGAGGCGCCGTGTTTTTCGTCGGCATGGCACTCTGGGG
CGCCCACCGACTGTATAGCCTCCGCAACTCCCCTACCAGCGTGCTGCCCTCTTTCT
ATCAGGCCATGGCCTGCCCCAATGAGGTCGTGTCTTACGAGATCGTGCTCAGCAT
CACCAGGCTGATTAAGAAGTACAGGAAGGAGCTGCAGGTGGTGGCCTGGGATAT
TCTGTTAAACATCATCGAGCGCCTGCTGCAGCAGCTGCAGACTCTGGACTCCCCT
GAGCTGCGGACCATTGTCCACGATCTGCTGACCACCGTGGAGGAGCTGTGCGAT
CAGAACGAATTCCACGGCTCTCAGGAGCGGTATTTTGAGCTGGTGGAGCGGTGC
GCCGACCAGAGACCTGAGTCATCACTGCTGAACCTGATCTCCTACAGGGCTCAGT
CCATCCACCCAGCCAAAGACGGCTGGATCCAGAACCTGCAGGCACTGATGGAGC
GCTTCTTCCGGTCTGAATCTCGGGGCGCCGTAAGGATCAAGGTGCTGGATGTGCT
GAGCTTCGTGCTGCTGATCAACCGGCAGTTCTACGAGGAGGAGCTGATCAACTCT
GTGGTGATCAGTCAGCTGTCTCATATCCCCGAGGATAAAGACCACCAGGTGAGA
AAGCTGGCCACCCAGCTGCTCGTGGATCTGGCCGAGGGCTGCCACACACACCAC
TTCAACAGCCTGCTGGACATCATCGAAAAAGTGATGGCCCGTTCCCTGAGCCCCC
CACCCGAGCTGGAAGAGCGCGATGTGGCCGCTTATTCTGCCAGCCTGGAAGACG
TGAAAACTGCCGTCCTGGGCCTGCTGGTGATTCTGCAGACCAAGCTGTATACATT
GCCAGCCAGTCACGCCACCAGGGTGTACGAGATGCTGGTGAGCCATATCCAGCT
CCACTATAAGCACTCTTATACCCTGCCAATTGCCAGCTCCATTAGGCTGCAGGCC
TTTGACTTCCTGCTGCTGCTGAGGGCTGACAGCCTGCACAGACTGGGACTGCCTA
ACAAGGATGGGGTGGTGCGCTTCAGCCCCTACTGCGTGTGCGGCAGCGGCTCTC
TGCTGTTTAGGGTGCTGCTGCAGTGTCTCAAACAGGAGAGCGACTGGAAAGTGC
TGAAACTGGTGCTGGGCCGCCTGCCCGAATCCCTGAGGTACAAAGTGCTGATCTT
CACATCCCCATGCTCCGTGGACCAGCTGTGTTCAGCCCTGTGCTCAATGCTGTCC
GGACCCAAAACACTGGAGAGACTGAGGGGCGCCCCAGAGGGCTTTTCCAGGACA
GACCTGCACCTGGCAGTGGTGCCTGTGTTGACCGCACTGATCTCCTATCACAACT
ATCTGGATAAGACCAAGCAGAGGGAGATGGTGTACTGCCTGGAGCAGGGACTGA
TTCACCGTTGCGCCAGCCAGTGCGTGGTCGCTCTGTCAATCTGCAGTGTGGAGAT
GCCCGATATCATCATCAAGGCTCTGCCTGTCCTGGTGGTGAAGCTGACACACATC
AGCGCCACCGCCAGCATGGCTGTGCCCCTGCTGGAGTTTCTGAGCACTCTGGCCC
GGCTGCCCCACCTGTACCGTAACTTTGCCGCCGAGCAGTACGCCTCCGTGTTTGC
CATTAGCTTGCCCTACACCAACCCATCCAAGTTCAATCAATATATCGTGTGCCTG
GCCCACCACGTGATCGCTATGTGGTTCATCAGATGCCGCCTCCCCTTCCGGAAGG
ATTTCGTGCCCTTTATCACCAAAGGGCTGAGGTCTAACGTGCTGCTCTCTGGGAG
CGGCAAGAATCTGCACCTGGAACTGACCGAGACCTGCCTGGACATGATGGCCAG
GTACGTGTTTTCCAACTTTACAGCCGTGCCTAAACGCTCCCCAGTCGGCGAATTT
CTGCTGGCCGGGGGCCGCACTAAGACCTGGCTCGTGGGCAACAAGCTCGTGACC
GTGACCACCAGCGTGGGTACTGGGACACGGTCCCTGCTGGGCGGCAGCGGAGG
CCTGGCCGCCTACGTGCCTCTGCTTACACAGGGCTGGGCCGAAATCCTGGTGCGA
CGCCCAACGGGAAACACCTCTTGGCTGATGTCCCTGGAGAATCCTGGAAGTGGT
ATCAATCCTAGCTTCGTGTTTCTGCAGCTGTACCACAGCCCTTTTTTCGGCGACGA
GTCCAATAAACCCATCCTGCTGCCTAACGAGTCCCAGAGCTTCGAGCGGTCAGTG
CAGCTTCTGGATCAGATCCCAAGCTACGACACACACAAGATCGCTGTGCTGTATG
TTGGCGAAGGCCAGAGTAATAGCGAGCTGGCCATCCTGAGCAATGAGCACGGCA
GCTACCGCTATACAGAGTTCCTGACCGGCCTCGGAAGGCTGATTGAGCTGAAGG
ATTGTCAGCCAGATAAGGTCTATCTGGGCGGCCTGGATGTGTGCGGCGAGGATG
GCCAGTTTACATATTGCTGGCACGATGATATCATGCAGGCCGTGTTCCACATCGC
CACACTGATGCCTACCAAAGATGTGGACAAGCACAGGTGCGACAAGAAAAGAC
ACCTGGGCAACGACTTCGTGTCCATCGTGTACAATGACAGTGGAGAGGACTTCA
AGCTGGGCACCATTAAGGGCCAGTTCAATTTCGTGCACGTTATTGTTACACCTCT
GGATTACGAATGCAACCTCGTGAGTCTGCAGTGTAGGAAGGACATGGAGGGCTT
GGTGGACACCTCTGTGGCCAAGATTGTGAGCGACAGAAATCTGCCCTTTGTGGCC
AGGCAGATGGCACTGCATGCCAATATGGCCTCTCAGGTGCACCACTCCCGGTCA
AATCCCACTGACATCTATCCCTCCAAGTGGATCGCCAGACTGAGACATATTAAGC
GCCTGCGCCAGCGAATCTGTGAGGAGGCCGCCTATTCCAACCCATCATGAacccagc
tttcttgtacaaagtgggaattcctagagctcgctgatcagcctcgactgtgccttctagttgccagccatctgttgtttgcccctcccc
cgtgccttccttgaccctggaaggtgccactcccactgtcctttcctaataaaatgaggaaattgcatcgcattgtctgagtaggt
gtcattctattctggggggtggggtggggcaggacagcaagggggaggattgggaagagaatagcaggcatgctggggag
ggccgcaggaacccctagtgatggagttggccactccctctctgcgcgctcgctcgctcactgaggccgggcgaccaaaggtcgcc
cgacgcccgggctttgcccgggcggcctcagtgagcgagcgagcgcgcag
SEQ ID NO: 41, Protein, Artificial sequence, Micro-Tuberin 9 protein sequence
MAKPTSKDSGLKEKFKILLGLGTPRPNPRSAEGKQTEFIITAEILRELSMECGLNNRIR
MIGQICEVAKTKKFEEHAVEALWKAVADLLQPERPLEARHAVLALLKAIVQGQGER
LGVLRALFFKVIKDYPSNEDLHERLEVFKALTDNGRHITYLEEELADFVLQWMDVG
LSSEFLLVLVNLVKFNSCYLDEYIARMVQMICLLCVRTASSVDIEVSLQVLDAVVCY
NCLPAESLPLFIVTLCRTINVKELCEPCWKLMRNLLGTHLGHSAIYNMCHLMEDRAY
MEDAPLLRGAVFFVGMALWGAHRLYSLRNSPTSVLPSFYQAMACPNEVVSYEIVLSI
TRLIKKYRKELQVVAWDILLNIIERLLQQLQTLDSPELRTIVHDLLTTVEELCDQNEFH
GSQERYFELVERCADQRPESSLLNLISYRAQSIHPAKDGWIQNLQALMERFFRSESRG
AVRIKVLDVLSFVLLINRQFYEEELINSVVISQLSHIPEDKDHQVRKLATQLLVDLAEG
CHTHHFNSLLDIIEKVMARSLSPPPELEERDVAAYSASLEDVKTAVLGLLVILQTKLY
TLPASHATRVYEMLVSHIQLHYKHSYTLPIASSIRLQAFDFLLLLRADSLHRLGLPNK
DGVVRFSPYCVCGSGSLLFRVLLQCLKQESDWKVLKLVLGRLPESLRYKVLIFTSPC
SVDQLCSALCSMLSGPKTLERLRGAPEGFSRTDLHLAVVPVLTALISYHNYLDKTKQ
REMVYCLEQGLIHRCASQCVVALSICSVEMPDIIIKALPVLVVKLTHISATASMAVPL
LEFLSTLARLPHLYRNFAAEQYASVFAISLPYTNPSKFNQYIVCLAHHVIAMWFIRCR
LPFRKDFVPFITKGLRSNVLLSGSGKNLHLELTETCLDMMARYVFSNFTAVPKRSPV
GEFLLAGGRTKTWLVGNKLVTVTTSVGTGTRSLLGGSGGLAAYVPLLTQGWAEILV
RRPTGNTSWLMSLENPGSGINPSFVFLQLYHSPFFGDESNKPILLPNESQSFERSVQLL
DQIPSYDTHKIAVLYVGEGQSNSELAILSNEHGSYRYTEFLTGLGRLIELKDCQPDKV
YLGGLDVCGEDGQFTYCWHDDIMQAVFHIATLMPTKDVDKHRCDKKRHLGNDFVS
IVYNDSGEDFKLGTIKGQFNFVHVIVTPLDYECNLVSLQCRKDMEGLVDTSVAKIVS
DRNLPFVARQMALHANMASQVHHSRSNPTDIYPSKWIARLRHIKRLRQRICEEAAYS
NPS
SEQ ID NO: 42, Protein, Artificial sequence, Linker sequence
GSSGSSGSSGSS
SEQ ID NO: 43, Protein, Artificial sequence, Linker sequence
GSG
SEQ ID NO: 44, Protein, Artificial sequence, Linker sequence
GGSGG
SEQ ID NO: 45, Protein, Artificial sequence, Linker sequence
GSSGSSGSSGSSGSSGS
SEQ ID NO: 46, Protein, Artificial sequence, Linker sequence
GSSGS
SEQ ID NO: 47, Protein, Artificial sequence, Linker sequence
GSSGSSGS
SEQ ID NO: 48, Protein, Artificial sequence, Linker sequence
GSSG
SEQ ID NO: 49, Protein, Artificial sequence, Linker sequence
GGSGGSGGS
SEQ ID NO: 50, DNA, Artificial sequence, Linker sequence
ggcagttcgggcagctccgggtcctccggctcctct
SEQ ID NO: 51, DNA, Artificial sequence, Linker sequence
ggatccgga
SEQ ID NO: 52, DNA, Artificial sequence, Linker sequence
ggcggcagcggaggg
SEQ ID NO: 53, DNA, Artificial sequence, Linker sequence
ggctcctctggtagctctggaagttccggctccageggctcctccggatct
SEQ ID NO: 54, DNA, Artificial sequence, Linker sequence
ggctccagtggcagc
SEQ ID NO: 55, DNA, Artificial sequence, Linker sequence
ggctcctccggctctagcggcagc
SEQ ID NO: 56, DNA, Artificial sequence, Linker sequence
ggcagctccgga
SEQ ID NO: 57, DNA, Artificial sequence, Linker sequence
ggtggaagcggcggcagtggaggctcc

### EQUIVALENTS

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

1. An engineered micro-Tuberin comprising, from the amino to the carboxy terminus:
(i) a hamartin interface domain,
(ii) a GTPase-activating protein (GAP) extension domain,
(iii) a first tuberin dimerization interface domain,
(iv) a second tuberin dimerization interface domain, and
(v) a GTPase-activating protein (GAP) domain,
wherein the total length of the engineered micro-Tuberin is less than about 1300 amino acids.

2. The engineered micro-Tuberin of claim 1, wherein the micro-Tuberin does not include unstructured regions of the wild type tuberin protein as shown by SEQ ID NO: 1.

3. The engineered micro-Tuberin of claim 2, wherein the unstructured regions comprise:
(a) amino acids 647-687 of SEQ ID NO: 1,
(b) amino acids 924-1015 of SEQ ID NO: 1,
(c) amino acids 1087-1182 of SEQ ID NO: 1,
(d) amino acids 1216-1493 of SEQ ID NO: 1, and
(e) amino acids 1765-1807 of SEQ ID NO: 1.

4. The engineered micro-Tuberin of claim 1, wherein the hamartin interface domain comprises:
(1) at least 85% sequence identity to amino acids 94-575 of SEQ ID NO: 1,
(2) at least 85% sequence identity to amino acids 94-575 and 580-646 of SEQ ID NO: 1,
(3) at least 85% sequence identity to each of amino acids 94-188, 230-267, 355-419, 459-520, and 580-646 of SEQ ID NO: 1,
(4) at least 85% sequence identity to amino acids 50-374, and 580-646 of SEQ ID NO: 1,
(5) at least 85% sequence identity to amino acids 271-646 of SEQ ID NO: 1,
(6) at least 85% sequence identity to amino acids 72-435 and 577-646 of SEQ ID NO: 1, or
(7) at least 85% sequence identity to amino acids 1-646 of SEQ ID NO: 1.

5. The engineered micro-Tuberin of claim 1, wherein the GTPase-activating protein (GAP) extension domain comprises:
(1) at least 85% sequence identity to amino acids 815-923 of SEQ ID NO: 1, or
(2) at least 85% sequence identity to amino acids 687-923 of SEQ ID NO: 1, and
wherein the first tuberin dimerization interface domain comprises at least 85% sequence identity to amino acids 1016-1086 of SEQ ID NO: 1, and
wherein the second tuberin dimerization interface domain comprises at least 85% sequence identity to amino acids 1183-1215 of SEQ ID NO: 1, and
wherein the GAP domain comprises at least 85% sequence identity to amino acids 1494-1764 of SEQ ID NO: 1.

6.-8. (canceled)

9. The engineered micro-Tuberin of claim 1, further comprising a flexible linker region between each of the domains (i)-(v), selected from any one of SEQ ID NOs: 42-49.

10. (canceled)

11. The engineered micro-Tuberin of claim 1, wherein the micro-Tuberin sequence comprises an amino acid sequence selected from SEQ ID NOs: 11, 16, 21, 26, 31, 36, or 41.

12. The engineered micro-Tuberin of claim 1, further comprising a detectable label.

13. A polynucleotide encoding the engineered micro-Tuberin of claim 1, or a complement thereof.

14. A vector comprising the polynucleotide of claim 13.

15. A polynucleotide comprising at least 85% sequence identity to a micro-Tuberin coding sequence selected from SEQ ID NOs: 7, 8, 12, 13, 17, 18, 22, 23, 27, 28, 32, 33, 37, or 38.

16. The polynucleotide of claim 15, further comprising a Kozak sequence (GCCACC) immediately 5′ of the micro-Tuberin coding sequence, and optionally further comprising at least one linker sequence selected from SEQ ID NOs: 50-57.

17. The polynucleotide of claim 15, further comprising an Inverted terminal repeat (ITR) sequence, a promoter sequence, a 3′ UTR sequence, a polyA sequence, and a 3′ Inverted terminal repeat (ITR) sequence.

18. The polynucleotide of claim 17, wherein the ITR sequence comprises SEQ ID NO: 2, wherein the promoter sequence comprises SEQ ID NO: 3, wherein 3′ UTR sequence comprises SEQ ID NO: 4, wherein the polyA sequence comprises SEQ ID NO: 5, and wherein the 3′ UTR sequence comprises SEQ ID NO: 6.

19. The polynucleotide of claim 15, comprising at least 85% sequence identity to a sequence selected from SEQ ID NOs: 9, 10, 14, 15, 19, 20, 24, 25, 29, 30, 34, 35, 39, or 40.

20. A vector comprising the polynucleotide of claim 15.

21. The vector of claim 14, wherein the vector is an AAV vector, and wherein the AAV vector comprises a capsid selected from AAV1, AAV2, AAV3, AAV4, AAV5, AAV6, AAV7, AAV8, AAV9, AAV10, AAV11, AAV12, AAV13 or AAV rh74 capsid, or a variant thereof.

22. A cell comprising the engineered micro-Tuberin of claim 1.

23. A composition comprising the engineered micro-Tuberin of claim claim 1.

24. A method for delivering a micro-Tuberin polynucleotide or polypeptide to a cell comprising contacting the cell with the engineered micro-Tuberin of claim 1, wherein the contacting is in vitro or in vivo.

25.-26. (canceled)

27. A method of treating a subject suffering from mTOR hyperactivation comprising administering to the subject the engineered micro-Tuberin of any one of claim 1.

28. The method of claim 27, wherein the disease is selected from the group consisting of tuberous sclerosis complex (TSC) type 2, focal cortical dysplasia type 2, lymphangioleiomyomatosis, and renal angiomyolipoma, or
wherein the administration is achieved systemically, intravascularly, intracerebrally, or intrathecally; or wherein the micro-Tuberin administration is into the renal artery or vein, or into the lungs, or
wherein the administration is achieved by an AAV vector, and wherein the AAV vector comprises a capsid selected from AAV1, AAV2, AAV3, AAV4, AAV5, AAV6, AAV7, AAV8, AAV9, AAV10, AAV11, AAV12, AAV13 or AAV rh74 capsid, or wherein the AAV vector is parenterally administered by injection, infusion or implantation.

29.-30. (canceled)

31. The method of claim 27, further administering an mTOR inhibitor; optionally wherein the mTOR inhibitor is selected from rapamycin, pimecrolimus, tacrolimus, sirolimus, temsirolimus, everolimus, ridaforolimus, samimod, AZD8055, PF04691502, SF1126, XL765, or pharmaceutically acceptable salts and combinations thereof, wherein the subject is a mammal, optionally a human.

32. (canceled)
